SEQUENCE LISTING (74 sequences)

SEQ ID NO: 1 - DNA, 270 bp, Artificial Sequence
  SP0497
tctgagggagacagggaggcatgatcactgccaaatgcccaccaaggacaaggcacatcccagggagacagacgcagacctggtgccctctggacactggcattcctggagttctcctctataaatacccgctctggtatttggggttggcagctgttgcggccggggccgcattcctgggggccgggcggtgctcccgcccgcctcgataaaaggctccggggccggcggcggcccacgagctacccggaggagcgggaggcggccacc

SEQ ID NO: 2 - DNA, 258 bp, Artificial Sequence
  SP0498
tctgagggagacagggaggcatgatcactgccaaatgcccaccaaggacaaggcacatcccagggagacagacgcagacctggtgccctctggacactggcattcctggagagggtcagtgtcctgccccaacccatgagatgacagactataatagccacaggattaacatagcaggcattgcaccgcctgctgccacggccggccgtataaatagaggcgaggagcagctgggctctcttggcagtcaccgccacc

SEQ ID NO: 3 - DNA, 234 bp, Artificial Sequence
  SP0499
tctgagggagacagggaggcatgatcactgccaaatgcccaccaaggacaaggcacatcccagggagacagacgcagacctggtgccctctggacactggcattcctggagttctcctctataaatacccgctctggtatttggggttggcagctgttgcaccgcctgctgccacggccggccgtataaatagaggcgaggagcagctgggctctcttggcagtcaccgccacc

SEQ ID NO: 4 - DNA, 267 bp, Artificial Sequence
  SP0500
ccagcccacctgtcccaatgctgacttagtgcaaggcgagccagcaaggagggaggacaggtggcagtggggggtgaggagcatctaaaaatagccccagcccacctgtcccaatgctgacttagtgcaaggcgagccagcaaggagggaggacaggtggcagtggggggtgaggagcatctaaaaatagcccaccgcctgctgccacggccggccgtataaatagaggcgaggagcagctgggctctcttggcagtcaccgccacc

SEQ ID NO: 5 - DNA, 219 bp, Artificial Sequence
  SP0501
ccagcccacctgtcccaatgctgacttagtgcaaggcgagccagcaaggagggaggacaggtggcagtggggggtgaggagcatctaaaaatagccttctcctctataaatacccgctctggtatttggggttggcagctgttgcaccgcctgctgccacggccggccgtataaatagaggcgaggagcagctgggctctcttggcagtcaccgccacc

SEQ ID NO: 6 - DNA, 251 bp, Artificial Sequence
  SP0502
ctagactagcatgctgcccatgtaaggaggcaaggcctggggacacccgagatgcctggttataattaacccagacatgtggctgcccccccccccccaacacctgctgcctctaaaaataaccctgcttctcctctataaatacccgctctggtatttggggttggcagctgttgcaccgcctgctgccacggccggccgtataaatagaggcgaggagcagctgggctctcttggcagtcaccgccacc

SEQ ID NO: 7 - DNA, 258 bp, Artificial Sequence
  SP0503
tcgtcccctggctggcccatgtaatctgagcccagcattgtacatatcctgggaacagctgacaatgcagtggtcagacagctggtggggccagctagagctggcagggttggctgggaggggagtgtaggctgattctcctctataaatacccgctctggtatttggggttggcagctgttgcaccgcctgctgccacggccggccgtataaatagaggcgaggagcagctgggctctcttggcagtcaccgccacc

SEQ ID NO: 8 - DNA, 265 bp, Artificial Sequence
  SP0504
ttggcccaggtcacactggggtgaggctagtgttcctgagccttgacaaggagacagcttgaaatagacgagtgtcacatttctgagcagctgtgtggcgacagcaggaggggtagggaatagacagtataaaagagaaagcttctcctctataaatacccgctctggtatttggggttggcagctgttgcaccgcctgctgccacggccggccgtataaatagaggcgaggagcagctgggctctcttggcagtcaccgccacc

SEQ ID NO: 9 - DNA, 269 bp, Artificial Sequence
  SP0505
ccctgccatcttgggtttcagggcagaggagtcttgctaattttgatgcctatttttggacacttcagctgccactggctccttataaacgcatgacaccccatgcaaacacactacccctccctccactgctgacaggtgtgtggttctcctctataaatacccgctctggtatttggggttggcagctgttgcaccgcctgctgccacggccggccgtataaatagaggcgaggagcagctgggctctcttggcagtcaccgccacc

SEQ ID NO: 10 - DNA, 274 bp, Artificial Sequence
  SP0506
aattatttttaataacacttactggtaagagaaaggggagaaaccttagacaggcacttagatgtgactaaggcaggtttatctctgattccaaagcactggagtggaagtcacaccgtgactcagagcattgtgatgggccagctgtccattctcctctataaatacccgctctggtatttggggttggcagctgttgcaccgcctgctgccacggccggccgtataaatagaggcgaggagcagctgggctctcttggcagtcaccgccacc

SEQ ID NO: 11 - DNA, 274 bp, Artificial Sequence
  SP0507
cagtgttcctatatttatcccaccatacagagcttcctttgcctcagaaggaccagcagtttcgctagcttaacaaaaccagccactcagggtattggtttacagtcaagcaactctgggagagggcagctgctctcagacatcatacagcttctcctctataaatacccgctctggtatttggggttggcagctgttgcaccgcctgctgccacggccggccgtataaatagaggcgaggagcagctgggctctcttggcagtcaccgccacc

SEQ ID NO: 12 - DNA, 240 bp, Artificial Sequence
  SP0508
cttctcaagccaaaggagcaagagttaaaaataacaggctcaccctggcagccacctgtgctggccagccccaccccatccctccctcggggacagctgcagctcctcaggccccgcccgggacattttgggaacactttctcctcttacttctcatcttcagggcaccgcctgctgccacggccggccgtataaatagaggcgaggagcagctgggctctcttggcagtcaccgccacc

SEQ ID NO: 13 - DNA, 275 bp, Artificial Sequence
  SP0509
agggtcagtgtcctgccccaacccatgagatgacagactataatagccacaggattaacatagcaggcattgatttttaaagactgaggaattaggcacctgtcatttttgccagctggtgtagatgttaaaaattactgtcactcttccgcctgctactttattttgcacctgctgttacttgagttacaggcatttcacaccgcctgctgccacggccggccgtataaatagaggcgaggagcagctgggctctcttggcagtcaccgccacc

SEQ ID NO: 14 - DNA, 251 bp, Artificial Sequence
  SP0510
atttttaaagactgaggaattaggcacctgtcatttttgccagctggtgtagatgttaaaaattactgtcactcttccgcctgctactttattttgcacctgctgttacttgagttacaggcatttcattctcctctataaatacccgctctggtatttggggttggcagctgttgcaccgcctgctgccacggccggccgtataaatagaggcgaggagcagctgggctctcttggcagtcaccgccacc

SEQ ID NO: 15 - DNA, 274 bp, Artificial Sequence
  SP0511
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagcttctcctctataaatacccgctctggtatttggggttggcagctgttggggctgggcataaaagtcagggcagagccatctattgcttacatttgcttctggccacc

SEQ ID NO: 16 - DNA, 218 bp, Artificial Sequence
  SP0512
tctgagggagacagggaggcatgatcactgccaaatgcccaccaaggacaaggcacatcccagggagacagacgcagacctggtgccctctggacactggcattcctggagttctcctctataaatacccgctctggtatttggggttggcagctgttggggctgggcataaaagtcagggcagagccatctattgcttacatttgcttctggccacc

SEQ ID NO: 17 - DNA, 275 bp, Artificial Sequence
  SP0513
ccagcccacctgtcccaatgctgacttagtgcaaggcgagccagcaaggagggaggacaggtggcagtggggggtgaggagcatctaaaaatagccagggtcagtgtcctgccccaacccatgagatgacagactataatagccacaggattaacatagcaggcattgttctcctctataaatacccgctctggtatttggggttggcagctgttggggctgggcataaaagtcagggcagagccatctattgcttacatttgcttctggccacc

SEQ ID NO: 18 - DNA, 251 bp, Artificial Sequence
  SP0514
ccagcccacctgtcccaatgctgacttagtgcaaggcgagccagcaaggagggaggacaggtggcagtggggggtgaggagcatctaaaaatagccttctcctctataaatacccgctctggtatttggggttggcagctgttgttctcctctataaatacccgctctggtatttggggttggcagctgttggggctgggcataaaagtcagggcagagccatctattgcttacatttgcttctggccacc

SEQ ID NO: 19 - DNA, 235 bp, Artificial Sequence
  SP0515
ctagactagcatgctgcccatgtaaggaggcaaggcctggggacacccgagatgcctggttataattaacccagacatgtggctgcccccccccccccaacacctgctgcctctaaaaataaccctgcttctcctctataaatacccgctctggtatttggggttggcagctgttggggctgggcataaaagtcagggcagagccatctattgcttacatttgcttctggccacc

SEQ ID NO: 20 - DNA, 242 bp, Artificial Sequence
  SP0516
tcgtcccctggctggcccatgtaatctgagcccagcattgtacatatcctgggaacagctgacaatgcagtggtcagacagctggtggggccagctagagctggcagggttggctgggaggggagtgtaggctgattctcctctataaatacccgctctggtatttggggttggcagctgttggggctgggcataaaagtcagggcagagccatctattgcttacatttgcttctggccacc

SEQ ID NO: 21 - DNA, 249 bp, Artificial Sequence
  SP0517
ttggcccaggtcacactggggtgaggctagtgttcctgagccttgacaaggagacagcttgaaatagacgagtgtcacatttctgagcagctgtgtggcgacagcaggaggggtagggaatagacagtataaaagagaaagcttctcctctataaatacccgctctggtatttggggttggcagctgttggggctgggcataaaagtcagggcagagccatctattgcttacatttgcttctggccacc

SEQ ID NO: 22 - DNA, 253 bp, Artificial Sequence
  SP0518
ccctgccatcttgggtttcagggcagaggagtcttgctaattttgatgcctatttttggacacttcagctgccactggctccttataaacgcatgacaccccatgcaaacacactacccctccctccactgctgacaggtgtgtggttctcctctataaatacccgctctggtatttggggttggcagctgttggggctgggcataaaagtcagggcagagccatctattgcttacatttgcttctggccacc

SEQ ID NO: 23 - DNA, 272 bp, Artificial Sequence
  SP0519
cttctcaagccaaaggagcaagagttaaaaataacaggctcaccctggcagccacctgtgctggccagccccaccccatccctccctcggggacagctgcagctcctcaggccccgcccgggacattttgggaacactttctcctcttacttctcatcttcagggttctcctctataaatacccgctctggtatttggggttggcagctgttggggctgggcataaaagtcagggcagagccatctattgcttacatttgcttctggccacc

SEQ ID NO: 24 - DNA, 259 bp, Artificial Sequence
  SP0520
agggtcagtgtcctgccccaacccatgagatgacagactataatagccacaggattaacatagcaggcattgatttttaaagactgaggaattaggcacctgtcatttttgccagctggtgtagatgttaaaaattactgtcactcttccgcctgctactttattttgcacctgctgttacttgagttacaggcatttcagggctgggcataaaagtcagggcagagccatctattgcttacatttgcttctggccacc

SEQ ID NO: 25 - DNA, 263 bp, Artificial Sequence
  SP0521
ctagactagcatgctgcccatgtaaggaggcaaggcctggggacacccgagatgcctggttataattaacccagacatgtggctgcccccccccccccaacacctgctgcctctaaaaataaccctgcttctcctctataaatacccgctctggtatttggggttggcagctgttggtacttatataagggggtgggggcgcgttcgtcctcagtcgcgatcgaacactcgagccgagcagacgtgcctacggaccggccacc

SEQ ID NO: 26 - DNA, 287 bp, Artificial Sequence
  SP4169
ctagactagcatgctgcccatgtaaggaggcaaggcctggggacacccgagatgcctggttataattaacccagacatgtggctgcccccccccccccaacacctgctgcctctaaaaataaccctgcttctcctctataaatacccgctctggtatttggggttggcagctgttgcggccggggccgcattcctgggggccgggcggtgctcccgcccgcctcgataaaaggctccggggccggcggcggcccacgagctacccggaggagcgggaggcggccacc

SEQ ID NO: 27 - DNA, 237 bp, Artificial Sequence
  SP0522
gctgcccatgtaaggaggcaaggcctggggacacccgagatgcctggttataattaacccagacatgtggctgccccccccccccaacacctgctgcctctaaaaataaccctgttctcctctataaatacccgctctggtatttggggttggcagctgttgcaccgcctgctgccacggccggccgtataaatagaggcgaggagcagctgggctctcttggcagtcaccgccacc

SEQ ID NO: 28 - DNA, 221 bp, Artificial Sequence
  SP0523
gctgcccatgtaaggaggcaaggcctggggacacccgagatgcctggttataattaacccagacatgtggctgccccccccccccaacacctgctgcctctaaaaataaccctgttctcctctataaatacccgctctggtatttggggttggcagctgttggggctgggcataaaagtcagggcagagccatctattgcttacatttgcttctggccacc

SEQ ID NO: 29 - DNA, 249 bp, Artificial Sequence
  SP0524
gctgcccatgtaaggaggcaaggcctggggacacccgagatgcctggttataattaacccagacatgtggctgccccccccccccaacacctgctgcctctaaaaataaccctgttctcctctataaatacccgctctggtatttggggttggcagctgttggtacttatataagggggtgggggcgcgttcgtcctcagtcgcgatcgaacactcgagccgagcagacgtgcctacggaccggccacc

SEQ ID NO: 30 - DNA, 159 bp, Artificial Sequence
  CRM from SP0497, SP0499 and SP0512
tctgagggagacagggaggcatgatcactgccaaatgcccaccaaggacaaggcacatcccagggagacagacgcagacctggtgccctctggacactggcattcctggagttctcctctataaatacccgctctggtatttggggttggcagctgttg

SEQ ID NO: 31 - DNA, 183 bp, Artificial Sequence
  CRM_SP0498
tctgagggagacagggaggcatgatcactgccaaatgcccaccaaggacaaggcacatcccagggagacagacgcagacctggtgccctctggacactggcattcctggagagggtcagtgtcctgccccaacccatgagatgacagactataatagccacaggattaacatagcaggcattg

SEQ ID NO: 32 - DNA, 192 bp, Artificial Sequence
  CRM_SP0500
ccagcccacctgtcccaatgctgacttagtgcaaggcgagccagcaaggagggaggacaggtggcagtggggggtgaggagcatctaaaaatagccccagcccacctgtcccaatgctgacttagtgcaaggcgagccagcaaggagggaggacaggtggcagtggggggtgaggagcatctaaaaatagcc

SEQ ID NO: 33 - DNA, 144 bp, Artificial Sequence
  CRM_SP0501
ccagcccacctgtcccaatgctgacttagtgcaaggcgagccagcaaggagggaggacaggtggcagtggggggtgaggagcatctaaaaatagccttctcctctataaatacccgctctggtatttggggttggcagctgttg

SEQ ID NO: 34 - DNA, 176 bp, Artificial Sequence
  CRM from SP0502, SP0515, SP0521 and SP4169
ctagactagcatgctgcccatgtaaggaggcaaggcctggggacacccgagatgcctggttataattaacccagacatgtggctgcccccccccccccaacacctgctgcctctaaaaataaccctgcttctcctctataaatacccgctctggtatttggggttggcagctgttg

SEQ ID NO: 35 - DNA, 183 bp, Artificial Sequence
  CRM from SP0503 and SP0516
tcgtcccctggctggcccatgtaatctgagcccagcattgtacatatcctgggaacagctgacaatgcagtggtcagacagctggtggggccagctagagctggcagggttggctgggaggggagtgtaggctgattctcctctataaatacccgctctggtatttggggttggcagctgttg

SEQ ID NO: 36 - DNA, 190 bp, Artificial Sequence
  CRM from SP0504 and SP0517
ttggcccaggtcacactggggtgaggctagtgttcctgagccttgacaaggagacagcttgaaatagacgagtgtcacatttctgagcagctgtgtggcgacagcaggaggggtagggaatagacagtataaaagagaaagcttctcctctataaatacccgctctggtatttggggttggcagctgttg

SEQ ID NO: 37 - DNA, 194 bp, Artificial Sequence
  CRM from SP0505 and SP0518
ccctgccatcttgggtttcagggcagaggagtcttgctaattttgatgcctatttttggacacttcagctgccactggctccttataaacgcatgacaccccatgcaaacacactacccctccctccactgctgacaggtgtgtggttctcctctataaatacccgctctggtatttggggttggcagctgttg

SEQ ID NO: 38 - DNA, 199 bp, Artificial Sequence
  CRM_SP0506
aattatttttaataacacttactggtaagagaaaggggagaaaccttagacaggcacttagatgtgactaaggcaggtttatctctgattccaaagcactggagtggaagtcacaccgtgactcagagcattgtgatgggccagctgtccattctcctctataaatacccgctctggtatttggggttggcagctgttg

SEQ ID NO: 39 - DNA, 199 bp, Artificial Sequence
  CRM_SP0507
cagtgttcctatatttatcccaccatacagagcttcctttgcctcagaaggaccagcagtttcgctagcttaacaaaaccagccactcagggtattggtttacagtcaagcaactctgggagagggcagctgctctcagacatcatacagcttctcctctataaatacccgctctggtatttggggttggcagctgttg

SEQ ID NO: 40 - DNA, 165 bp, Artificial Sequence
  CRM_SP0508
cttctcaagccaaaggagcaagagttaaaaataacaggctcaccctggcagccacctgtgctggccagccccaccccatccctccctcggggacagctgcagctcctcaggccccgcccgggacattttgggaacactttctcctcttacttctcatcttcaggg

SEQ ID NO: 41 - DNA, 200 bp, Artificial Sequence
  CRM from SP0509 and SP0520
agggtcagtgtcctgccccaacccatgagatgacagactataatagccacaggattaacatagcaggcattgatttttaaagactgaggaattaggcacctgtcatttttgccagctggtgtagatgttaaaaattactgtcactcttccgcctgctactttattttgcacctgctgttacttgagttacaggcatttca

SEQ ID NO: 42 - DNA, 176 bp, Artificial Sequence
  CRM_SP0510
atttttaaagactgaggaattaggcacctgtcatttttgccagctggtgtagatgttaaaaattactgtcactcttccgcctgctactttattttgcacctgctgttacttgagttacaggcatttcattctcctctataaatacccgctctggtatttggggttggcagctgttg

SEQ ID NO: 43 - DNA, 215 bp, Artificial Sequence
  CRM_SP0511
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagcttctcctctataaatacccgctctggtatttggggttggcagctgttg

SEQ ID NO: 44 - DNA, 216 bp, Artificial Sequence
  CRM_SP0513
ccagcccacctgtcccaatgctgacttagtgcaaggcgagccagcaaggagggaggacaggtggcagtggggggtgaggagcatctaaaaatagccagggtcagtgtcctgccccaacccatgagatgacagactataatagccacaggattaacatagcaggcattgttctcctctataaatacccgctctggtatttggggttggcagctgttg

SEQ ID NO: 45 - DNA, 192 bp, Artificial Sequence
  CRM_SP0514
ccagcccacctgtcccaatgctgacttagtgcaaggcgagccagcaaggagggaggacaggtggcagtggggggtgaggagcatctaaaaatagccttctcctctataaatacccgctctggtatttggggttggcagctgttgttctcctctataaatacccgctctggtatttggggttggcagctgttg

SEQ ID NO: 46 - DNA, 213 bp, Artificial Sequence
  CRM_SP0519
cttctcaagccaaaggagcaagagttaaaaataacaggctcaccctggcagccacctgtgctggccagccccaccccatccctccctcggggacagctgcagctcctcaggccccgcccgggacattttgggaacactttctcctcttacttctcatcttcagggttctcctctataaatacccgctctggtatttggggttggcagctgttg

SEQ ID NO: 47 - DNA, 162 bp, Artificial Sequence
  CRM from SP0522, SP0523 and SP0524
gctgcccatgtaaggaggcaaggcctggggacacccgagatgcctggttataattaacccagacatgtggctgccccccccccccaacacctgctgcctctaaaaataaccctgttctcctctataaatacccgctctggtatttggggttggcagctgttg

SEQ ID NO: 48 - DNA, 151 bp, Artificial Sequence
  CRE0119
aattatttttaataacacttactggtaagagaaaggggagaaaccttagacaggcacttagatgtgactaaggcaggtttatctctgattccaaagcactggagtggaagtcacaccgtgactcagagcattgtgatgggccagctgtcca

SEQ ID NO: 49 - DNA, 142 bp, Artificial Sequence
  CRE0127
ttggcccaggtcacactggggtgaggctagtgttcctgagccttgacaaggagacagcttgaaatagacgagtgtcacatttctgagcagctgtgtggcgacagcaggaggggtagggaatagacagtataaaagagaaagc

SEQ ID NO: 50 - DNA, 146 bp, Artificial Sequence
  CRE0137
ccctgccatcttgggtttcagggcagaggagtcttgctaattttgatgcctatttttggacacttcagctgccactggctccttataaacgcatgacaccccatgcaaacacactacccctccctccactgctgacaggtgtgtgg

SEQ ID NO: 51 - DNA, 165 bp, Artificial Sequence; identical to SEQ ID NO: 40
  CRE0138

SEQ ID NO: 52 - DNA, 151 bp, Artificial Sequence
  CRE0139
cagtgttcctatatttatcccaccatacagagcttcctttgcctcagaaggaccagcagtttcgctagcttaacaaaaccagccactcagggtattggtttacagtcaagcaactctgggagagggcagctgctctcagacatcatacagc

SEQ ID NO: 53 - DNA, 135 bp, Artificial Sequence
  CRE0143
tcgtcccctggctggcccatgtaatctgagcccagcattgtacatatcctgggaacagctgacaatgcagtggtcagacagctggtggggccagctagagctggcagggttggctgggaggggagtgtaggctga

SEQ ID NO: 54 - DNA, 114 bp, Artificial Sequence
  CRE0145
gctgcccatgtaaggaggcaaggcctggggacacccgagatgcctggttataattaacccagacatgtggctgccccccccccccaacacctgctgcctctaaaaataaccctg

SEQ ID NO: 55 - DNA, 111 bp, Artificial Sequence
  CRE0077
tctgagggagacagggaggcatgatcactgccaaatgcccaccaaggacaaggcacatcccagggagacagacgcagacctggtgccctctggacactggcattcctggag

SEQ ID NO: 56 - DNA, 48 bp, Artificial Sequence
  DES_MT_enhancer_48bp
ttctcctctataaatacccgctctggtatttggggttggcagctgttg

SEQ ID NO: 57 - DNA, 72 bp, Artificial Sequence
  CRE0075
agggtcagtgtcctgccccaacccatgagatgacagactataatagccacaggattaacatagcaggcattg

SEQ ID NO: 58 - DNA, 96 bp, Artificial Sequence
  CRE0083
ccagcccacctgtcccaatgctgacttagtgcaaggcgagccagcaaggagggaggacaggtggcagtggggggtgaggagcatctaaaaatagcc

SEQ ID NO: 59 - DNA, 128 bp, Artificial Sequence
  Ch2EnhMYL1_3_v1
atttttaaagactgaggaattaggcacctgtcatttttgccagctggtgtagatgttaaaaattactgtcactcttccgcctgctactttattttgcacctgctgttacttgagttacaggcatttca

SEQ ID NO: 60 - DNA, 128 bp, Artificial Sequence
  CRE0050
ctagactagcatgctgcccatgtaaggaggcaaggcctggggacacccgagatgcctggttataattaacccagacatgtggctgcccccccccccccaacacctgctgcctctaaaaataaccctgc

SEQ ID NO: 61 - DNA, 167 bp, Artificial Sequence
  CRE0069
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagc

SEQ ID NO: 62 - DNA, 53 bp, Artificial Sequence
  BG_mp
gggctgggcataaaagtcagggcagagccatctattgcttacatttgcttctg

SEQ ID NO: 63 - DNA, 81 bp, Artificial Sequence
  SCP1
gtacttatataagggggtgggggcgcgttcgtcctcagtcgcgatcgaacactcgagccgagcagacgtgcctacggaccg

SEQ ID NO: 64 - DNA, 105 bp, Artificial Sequence
  CRE0070
cggccggggccgcattcctgggggccgggcggtgctcccgcccgcctcgataaaaggctccggggccggcggcggcccacgagctacccggaggagcgggaggcg

SEQ ID NO: 65 - DNA, 69 bp, Artificial Sequence
  CRE0053
caccgcctgctgccacggccggccgtataaatagaggcgaggagcagctgggctctcttggcagtcacc

SEQ ID NO: 66 - DNA, 392 bp, Artificial Sequence
  SP0321
taagtccgggcagggtcctgtccataaaaggcttttcccgggccggctccccgccggcagcgtgccccgccccggcccgctccatctccaaagcatgcagagaatgtctcggcagccccggtagactgctccaacttggtgtctttccccaaatatggagcctgtgtggagtcactgggggagccgggggtggggagcggagccggcttcctctagaggtccctatatggttgtgttagagtgaacggccagcttcagcccgtctttgctccttgtttgggaagcgagtgggaggggatcagagcaaggggctatataacccttcagcgttcagcctcccgggacaccacccacccagagtggagaagcccagccagtcgctgtcagccacc

SEQ ID NO: 67 - DNA, 216 bp, Artificial Sequence
  CRE0031
taagtccgggcagggtcctgtccataaaaggcttttcccgggccggctccccgccggcagcgtgccccgccccggcccgctccatctccaaagcatgcagagaatgtctcggcagccccggtagactgctccaacttggtgtctttccccaaatatggagcctgtgtggagtcactgggggagccgggggtggggagcggagccggcttcctctag

SEQ ID NO: 68 - DNA, 176 bp, Artificial Sequence
  CRE0037
aggtccctatatggttgtgttagagtgaacggccagcttcagcccgtctttgctccttgtttgggaagcgagtgggaggggatcagagcaaggggctatataacccttcagcgttcagcctcccgggacaccacccacccagagtggagaagcccagccagtcgctgtcagccacc

SEQ ID NO: 69 - DNA, 18 bp, Artificial Sequence
  Luc2 forward primer
acgctgggctacttgatc

SEQ ID NO: 70 - DNA, 19 bp, Artificial Sequence
  Luc2 reverse primer
caagaatagctcctcctcg

SEQ ID NO: 71 - DNA, 19 bp, Artificial Sequence
  mGapdH forward primer
acggcaaattcaacggcac

SEQ ID NO: 72 - DNA, 20 bp, Artificial Sequence
  mGapdH reverse primer
tagtggggtctcgctcctgg

SEQ ID NO: 73 - DNA, 18 bp, Artificial Sequence
  Luc2 probe
tttcgggtcgtgctcatg

SEQ ID NO: 74 - DNA, 24 bp, Artificial Sequence
  mGapdH probe
ttgtcatcaacgggaagcccatca

(19) United States
(12) Patent Application Publication
(10) Pub. No.: US 2025/0129381 A1
(43) Pub. Date: Apr. 24, 2025

(54) REGULATORY NUCLEIC ACID SEQUENCES

(71) Applicant: ASKLEPIOS BIOPHARMACEUTICAL, INC., Research Triangle Park, NC (US)

(72) Inventors: Jorge Omar YANEZ-CUNA, Midlothian (GB); Juan Manuel IGLESIAS, Midlothian (GB); Sinclair COOPER, Midlothian (GB); Michael L. ROBERTS, Midlothian (GB); Antonia EVRIPIOTI, Midlothian (GB)

(73) Assignee: ASKLEPIOS BIOPHARMACEUTICAL, INC., Research Triangle Park, NC (US)

(21) Appl. No.: 18/572,668

(22) PCT Filed: Jun. 23, 2022
(86) PCT No.: PCT/GB2022/051611

§ 371 Date: Dec. 20, 2023

(30) Foreign Application Priority Data
Jun. 23, 2021 (GB) 2108997.4
Aug. 5, 2021 (GB) 2111317.0

(51) Int. Cl. C12N 15/86; A61K 48/00

(52) U.S. Cl. CPC C12N 15/86; A61K 48/0058; C12N 2750/14143; C12N 2830/008

(57) ABSTRACT

The present invention relates to regulatory nucleic acid sequences, in particular muscle-specific promoters, elements thereof, and other such nucleic acid sequences, that are capable of enhancing muscle-specific expression of genes. The invention also relates to expression constructs, vectors and cells comprising such muscle-specific regulatory nucleic acid sequences, and to methods of their use. The regulatory nucleic acid sequences are of particular utility for gene therapy applications, but also find utility in other areas such as bioprocessing and biotechnology.

### CROSS REFERENCE TO RELATED APPLICATIONS

This application is a 35 U.S.C. § 371 National Phase Entry Application of International Application No. PCT/GB2022/051611 filed Jun. 23, 2022, which claims benefit under 35 U.S.C. § 119 (b) of GB Application Nos. 2108997.4 filed Jun. 23, 2021, and 2111317.0 filed Aug. 5, 2021, the contents of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Jun. 29, 2022, is named pctgb2022051611-seql and is 29 KB in size.

### FIELD OF THE INVENTION

The present invention relates to regulatory nucleic acid sequences, in particular muscle-specific synthetic promoters, elements thereof, and other such nucleic acid sequences, that are capable of enhancing muscle-specific expression of genes. The invention also relates to expression constructs, vectors and cells comprising such muscle-specific regulatory nucleic acid sequences, and to methods of their use. The regulatory nucleic acid sequences are of particular utility for gene therapy applications, but also find utility in other areas such as bioprocessing and biotechnology.

### BACKGROUND OF THE INVENTION

The following discussion is provided to aid the reader in understanding the disclosure and does not constitute any admission as to the contents or relevance of the prior art.

In many areas, including gene therapy, it is desirable to provide regulatory nucleic acid sequences that are capable of driving expression of a gene to produce a protein or nucleic acid expression product within a desired cell, tissue or organ.

Expression of therapeutic genes in muscle is attractive for gene therapy. Gene therapy in muscle has the potential to correct or augment the expression of various muscle proteins, such as dystrophin and sarcoglycans. This can be used to treat conditions such as muscular dystrophy, e.g. Duchenne muscular dystrophy (DMD). Muscle can also be used as a platform to express therapeutic protein for the treatment of other conditions such as congestive heart failure.

Various vectors have been used to deliver genes to muscle cells, for example adenoviral, retroviral, lentiviral, and adeno-associated viral (AAV), as well as non-viral vectors such as plasmids. Adenoviral vectors have a comparatively large cloning capacity and can transduce some cells efficiently. However, they face significant challenges in view of the strong immune response they tend to elicit. Retroviral and lentiviral vectors stably integrate into the genome, which is associated with both benefits and disadvantages. Lentiviral vectors can transduce both dividing and non-dividing cells, but most conventional retroviral vectors can only transduce dividing cells, which limits their use in non-dividing muscle cells. Plasmid DNA can be used to transfer genes to muscle cell in vitro, but their potential utility in a clinical context is less clear. AAV vectors are particularly attractive for gene therapy applications in muscle. AAV vectors display a natural tropism to muscle cells, can drive long-term expression of a therapeutic payload, and elicit a minimal immune response. Despite the ability of some gene therapy vectors to preferentially transduce muscle cells, off-target transduction does occur. Several Phase 1 and Phase 2 clinical trials using AAV serotypes 1, 2 and chimeric 2.5 have been reported for the treatment of Duchenne muscular dystrophy (DMD) and alpha-1 antitrypsin deficiency (D. E. Bowles, S. W J McPhee, C. Li, S. J. Gray, J. J. Samulski, A. S. Camp, J. Li, B. Wang, P. E. Monahan, J. E. Rabinowitz, J. C. Grieger, La. Govindasamy, M. Agbandje-McKenna, X. Xiao and R. J. Samulski, Phase 1 gene therapy for Duchenne Muscular Dystrophy using a translational optimised AAV vector. Molecular Therapy, 20, 443-455 (2012); M. L. Brantly, J. D. Chulay, L. Wang, C. Mueller, M. Humphries, L. T. Spencer, F. Rouhani, T. J. Conlon, R. Calcedo, M. R. Berts, C. Spencer, B. J. Byrne, J. M. Wilson, T. R. Flotte, Sustained transgene expression despite T lymphocyte responses in a clinical trial of rAAVI-AAT gene therapy. Proceedings of the National Academy of Sciences of the United States of America 106, 16363-16368 (2009); T. R. Flotte, M. L. Brantly, L. T. Spencer, B. J. Byrne, C. T. Spencer, D. J. Baker, M. Humphries, 15 Phase I trial of intramuscular injection of a recombinant adeno-associated virus alpha 1-antitrypsin (rAAV2-CB-hAAT) gene vector to AAT-deficient adults. Human gene therapy 15, 93-128 (2004); T. R. Flotte, B. C. Trapnell, M. Humphries, B. Carey, R. Calcedo, F. Rouhani, M. Campbell-Thompson, A. T. Yachnis, R. A. Sandhaus, N. G. McElvaney, C. Mueller, L. M. Messina, J. M. Wilson, M. Brantly, D. R. Knop, G. J. Ye, J. D. Chulay, Phase 2 clinical trial of a recombinant adeno-associated viral vector expressing alphal-antitrypsin: interim results. Human gene therapy 22, 1239-1247 (2011); C. Mueller, J. D. Chulay, B. C. Trapnell, M. Humphries, B. Carey, R. A. Sandhaus, N. G. McElvaney, L. Messina, Q. Tang, F. N. Rouhani, M. Campbell-Thompson, A. D. Fu, A. Yachnis, D. R. Knop, G. J. Ye, M. Brantly, R. Calcedo, S. Somanathan, L. P. Richman, R. H. Vonderheide, M. A. Hulme, T. M. Brusko, J. M. Wilson, T. R. Flotte, Human Treg responses allow sustained recombinant adeno-associated virus-mediated transgene expression. The Journal of clinical investigation 123, 5310-5318 (2013)).

It is desirable to provide systems to regulate gene expression in a muscle-specific manner. Ideally, such systems are highly-specific to the muscle (thereby avoiding or minimising off-target expression in non-target tissues) and are also powerful, i.e. they drive high expression levels in the muscle. Further, it may be desirable to provide systems to regulate gene expression in predominantly skeletal muscle-specific manner or predominantly cardiac muscle-specific manner. Such systems may be incorporated in expression constructs and vectors for specific expression of a desired gene for the treatment of skeletal muscle or cardiac muscle diseases or disorders. The use of cis-acting regulatory elements has been proposed to provide both specificity and activity. Typically, this concerns cis-regulatory enhancer sequences, i.e. nucleic acid sequences that act in cis to increase the activity of a promoter.

Various muscle specific promoters are known in the art, typically obtained from genes that are expressed predominantly in the muscle, for example, such as those encoding for desmin, skeletal actin, heart α-actin, muscle creatine kinase (CKM), myosin heavy and light chains, and troponin T/I. The C5-12 promoter represents a known synthetic promoter.

Regulatory sequences of short length are particularly desirable to minimise the proportion of a gene therapy vector taken up by regulatory sequences; this is particularly important for gene therapy vectors with limited capacity (payload) such as AAV vectors. Furthermore, while it is desirable to provide promoters that are powerful, in many instances it may be desirable for the skilled person to be able to select a suitable promoter having a desired power, e.g. from a range of promoters of varying powers.

There remains a need in the art for regulatory nucleic acids which are able to drive muscle-specific gene expression. In particular, there is a need for muscle-specific regulatory sequences of short size (e.g. promoters, cis-regulatory modules, cis-regulatory elements and minimal or proximal promoter elements) which can be incorporated in expression constructs and vectors for muscle-specific expression of a desired gene (e.g. a therapeutic transgene in a gene therapy context). Moreover, there is a need for muscle-specific regulatory sequences of short size which are primarily active in skeletal muscle or cardiac muscle which can be incorporated in expression constructs and vectors for skeletal muscle-specific expression or cardiac muscle-specific expression of a desired gene.

### SUMMARY OF THE INVENTION

In a first aspect of the present invention, there is provided:
a) a synthetic muscle-specific promoter comprising or consisting of a sequence according to any one of SEQ ID NOs: 1-29, 66 or a functional variant thereof; or
b) a synthetic muscle-specific promoter comprising or consisting of a cis-regulatory module (CRM) comprising a sequence according to any one of SEQ ID NOs: 30-47, or a functional variant thereof.

In some embodiments the synthetic muscle-specific promoter comprises a sequence which is at least 70%, 75%, 80%, 85%, 90%, 91%, 92%, 93%, 94%, 95%, 96%, 97%, 98% or 99% identical to any one of SEQ ID NOs: 1-29, 66.

In some embodiments the synthetic muscle-specific CRM comprises a sequence which is at least 70%, 75%, 80%, 85%, 90%, 95%, 96%, 97%, 98% or 99% identical to any one of SEQ ID NOs: 30-47. In some embodiments, a synthetic muscle-specific cis-regulatory module (CRM) according to the present invention comprises two or more operably linked cis-regulatory elements (CREs) selected from the group consisting of:
CRE0119 (SEQ ID NO: 48) or a functional variant thereof;
CRE0127 (SEQ ID NO: 49) or a functional variant thereof;
CRE0137 (SEQ ID NO: 50) or a functional variant thereof;
CRE0138 (SEQ ID NO: 51) or a functional variant thereof;
CRE0139 (SEQ ID NO: 52) or a functional variant thereof;
CRE0143 (SEQ ID NO: 53) or a functional variant thereof;
CRE0145 (SEQ ID NO: 54) or a functional variant thereof;
CRE0077 (SEQ ID NO: 55) or a functional variant thereof;
DES_MT_enhancer_48 bp (SEQ ID NO: 56) or a functional variant thereof;
CRE0075 (SEQ ID NO: 57) or a functional variant thereof;
CRE0083 (SEQ ID NO: 58) or a functional variant thereof;
Ch2EnhMYL1_3_v1 (SEQ ID NO: 59) or a functional variant thereof;
CRE0050 (SEQ ID NO: 60) or a functional variant thereof;
CRE0031 (SEQ ID NO: 67) or a functional variant thereof; and
CRE0069 (SEQ ID NO: 61) or a functional variant thereof.

In some embodiments the synthetic muscle-specific promoter according to b) comprises a CRM as set out above operably linked to a promoter element (typically a minimal or proximal promoter). The proximal promoter is preferably a muscle-specific proximal promoter.

In some embodiments, a synthetic muscle-specific synthetic promoter according to the present invention comprises at least one cis-regulatory element (CRE) selected from the group consisting of:
CRE0119 (SEQ ID NO: 48) or a functional variant thereof;
CRE0127 (SEQ ID NO: 49) or a functional variant thereof;
CRE0137 (SEQ ID NO: 50) or a functional variant thereof;
CRE0138 (SEQ ID NO: 51) or a functional variant thereof;
CRE0139 (SEQ ID NO: 52) or a functional variant thereof;
CRE0143 (SEQ ID NO: 53) or a functional variant thereof;
CRE0145 (SEQ ID NO: 54) or a functional variant thereof;
CRE0077 (SEQ ID NO: 55) or a functional variant thereof;
DES_MT_enhancer_48 bp (SEQ ID NO: 56) or a functional variant thereof;
CRE0075 (SEQ ID NO: 57) or a functional variant thereof;
CRE0083 (SEQ ID NO: 58) or a functional variant thereof;
Ch2EnhMYL1_3_v1 (SEQ ID NO: 59) or a functional variant thereof;
CRE0050 (SEQ ID NO: 60) or a functional variant thereof;
CRE0031 (SEQ ID NO: 67) or a functional variant thereof; and
CRE0069 (SEQ ID NO: 61) or a functional variant thereof operably linked to at least one promoter element selected from the group consisting of:
BG_mp (SEQ ID NO: 62) or a functional variant thereof;
SCP1 (SEQ ID NO: 63) or a functional variant thereof;
CRE0070 (SEQ ID NO: 64) or a functional variant thereof;
CRE0037 (SEQ ID NO: 68) or a functional variant thereof; and
CRE0053 (SEQ ID NO: 65) or a functional variant thereof.

The present invention thus provides various synthetic muscle-specific promoters and functional variants thereof. It is generally preferred that a synthetic promoter according to the present invention which is a variant of any one of SEQ ID NOs: 1-29, 66 retains at least 25%, 50%, 75%, 80%, 85%, 90%, 95% or 100% of the activity of the reference promoter.

Suitably said activity is assessed using one of the examples as described herein, but other methods can be used.

In another aspect of the present invention, there is provided a muscle-specific cis-regulatory element (CRE) comprising or consisting of a sequence according to any one of SEQ ID NOs: 48-61, 67 or a functional variant of any thereof. In some embodiments the muscle-specific CRE comprises a sequence which is at least 70%, 75%, 80%, 85%, 90%, 91%, 92%, 93%, 94%, 95%, 96%, 97%, 98% or 99% identical or any points therein to any one of SEQ ID NOs: 48-61, or 67.

It is generally preferred that a muscle-specific CRE according to the present invention which is a variant of any one of SEQ ID NOs: 48-61, or 67 retains at least 25%, 50%, 75%, 80%, 85%, 90%, 95% or 100% of the activity of the reference CRE. Suitably said activity is assessed using one of the examples as described herein, but other methods can be used.

In another aspect of the present invention there is provided a synthetic promoter comprising a CRE of any aspect of the present invention.

In another aspect of the present invention there is provided a CRM comprising a sequence according to any one of SEQ ID NOs: 30-47, or a functional variant thereof. In some embodiments the muscle-specific CRM comprises a sequence which is at least 70%, 75%, 80%, 85%, 90%, 91%, 92%, 93%, 94%, 95%, 96%, 97%, 98% or 99% identical or any points therein to any one of SEQ ID NOs: 30-47.

In some embodiments, the CRM comprises two or more operably linked cis-regulatory elements (CREs) selected from the group consisting of:
CRE0119 (SEQ ID NO: 48) or a functional variant thereof;
CRE0127 (SEQ ID NO: 49) or a functional variant thereof;
CRE0137 (SEQ ID NO: 50) or a functional variant thereof;
CRE0138 (SEQ ID NO: 51) or a functional variant thereof;
CRE0139 (SEQ ID NO: 52) or a functional variant thereof;
CRE0143 (SEQ ID NO: 53) or a functional variant thereof;
CRE0145 (SEQ ID NO: 54) or a functional variant thereof;
CRE0077 (SEQ ID NO: 55) or a functional variant thereof;
DES_MT_enhancer_48 bp (SEQ ID NO: 56) or a functional variant thereof;
CRE0075 (SEQ ID NO: 57) or a functional variant thereof;
CRE0083 (SEQ ID NO: 58) or a functional variant thereof;
Ch2EnhMYL1_3_v1 (SEQ ID NO: 59) or a functional variant thereof;
CRE0050 (SEQ ID NO: 60) or a functional variant thereof;
CRE0031 (SEQ ID NO: 67) or a functional variant thereof; and
CRE0069 (SEQ ID NO: 61) or a functional variant thereof.

In a further aspect of the present invention there is provided a minimal or proximal promoter comprising or consisting of a sequence according to any one of SEQ ID NOs: 62-65, 68 or a functional variant thereof. In another aspect of the present invention there is provided a synthetic promoter comprising said minimal or proximal promoter, suitably a synthetic muscle-specific promoter comprising said minimal or proximal promoter. Suitably a functional variant comprises a sequence which is at least 70%, 75%, 80%, 85%, 90%, 91%, 92%, 93%, 94%, 95%, 96%, 97%, 98% or 99% identical to SEQ ID NOs: 62-65, or 68. As indicated above, a ‘promoter element’ may be used to refer to a minimal or proximal promoter.

The CREs, CRMs, minimal/proximal promoters and synthetic promoters of the present invention can be active in various muscle tissues, particularly but not exclusively in skeletal muscle and/or cardiac muscle. CREs, CRMs, promoter elements or synthetic promoters which are active in at least one muscle tissue type or at least one muscle cell type may be referred to as ‘muscle-specific’. For ease, muscle-specific CREs, CRMs, promoter elements or synthetic promoters can be further subdivided in subtypes depending on whether the CREs, CRMs, promoter elements or synthetic promoters are predominantly active in skeletal or cardiac muscle.

In some embodiments the cis-regulatory elements, CRMs, promoter elements and synthetic promoters of the present invention are skeletal muscle-specific. Cis-regulatory elements, CRMs, promoter elements and synthetic promoters active predominantly in skeletal muscle and less active or not active in cardiac muscle are called ‘skeletal muscle-specific’. Non-limiting examples of skeletal muscle are quadriceps, diaphragm, tibialis anterior and soleus.

In some embodiments the cis-regulatory elements, CRMs, promoter elements and synthetic promoters of the present invention are cardiac muscle-specific. Cis-regulatory elements, CRMs, promoter elements and synthetic promoters of the present invention active predominantly in cardiac muscle and less active or not active in skeletal muscles are called ‘cardiac muscle-specific’.

In some embodiments the cis-regulatory elements, CRMs, promoter elements and synthetic promoters of the present invention are both skeletal and cardiac muscle specific.

In some embodiments, skeletal muscle-specific CREs, CRMs, promoter elements and synthetic promoters may be preferred. These CREs, CRMs, promoter elements and synthetic promoters may be preferred when promoter activity is required in the skeletal muscle with little or no activity in the heart (in the cardiac muscles). Examples of synthetic muscle-specific promoters designed to be predominantly active in skeletal muscle (skeletal muscle-specific synthetic promoters) include SP0497, SP0498, SP0499, SP0500, SP0501, SP0502, SP0503, SP0504, SP0505, SP0506, SP0507, SP0508, SP0509, SP0510, SP0511, SP0512, SP0513, SP0514, SP0515, SP0516, SP0517, SP0518, SP0519, SP0520, SP0521, SP0522, SP4169, SP0523 and SP0524. Examples of preferred synthetic skeletal muscle-specific promoters are SP0498, SP0500, SP0505, SP0508, SP0509, SP0513, SP0519, SP0522 and SP0524. The skeletal muscle-specific promoters may be active in fast-twitch muscles and/or slow-twitch muscles. In some embodiments, skeletal muscle-specific CREs, CRMs, promoter elements and synthetic promoters active in fast-twitch muscles may be preferred. In some embodiments, skeletal muscle-specific CREs, CRMs, promoter elements and synthetic promoters active in slow-twitch muscles may be preferred. In some embodiments, skeletal muscle-specific CREs, CRMs, promoter elements and synthetic promoters active both in slow-twitch and fast-twitch muscles may be preferred. Examples of skeletal muscle-specific promoters designed to be active in slow-twitch muscles are SP0500, SP0501 and SP0514.

The skeletal muscle-specific promoters may be active primarily in skeletal muscle and have low activity in cardiac muscle. Examples of synthetic muscle-specific promoters designed to be predominantly active in skeletal muscle but also expected to have low activity in cardiac muscle include SP0497, SP0498, SP0499 and SP0512.

The skeletal muscle-specific promoters may be active primarily in skeletal muscle and have activity in cardiac muscle. Examples of synthetic muscle-specific promoters designed to be predominantly active in skeletal muscle but also expected to have activity in cardiac muscle include SP0502, SP0515, SP0521, SP4169, SP0522, SP0523 and SP0524.

In some embodiments, synthetic muscle-specific promoters comprising or consisting of a sequence according to any one of SEQ ID NOs: 1 (SP0497), SEQ ID NO: 4 (SP0500), SEQ ID NO: 5 (SP0501), SEQ ID NO: 10 (SP0506), SEQ ID NO: 12 (SP0508), SEQ ID NO: 14 (SP0510), SEQ ID NO: 18 (SP0514), SEQ ID NO: 23 (SP0519), SEQ ID NO: 24 (SP0520), SEQ ID NO: 25 (SP0521) and SEQ ID NO: 26 (SP4169) are particularly preferred. In some embodiments, synthetic muscle-specific promoters comprising or consisting of a sequence according to any one of SEQ ID NO: 4 (SP0500), SEQ ID NO: 14 (SP0510), SEQ ID NO: 18 (SP0514) and SEQ ID NO: 23 (SP0519) are particularly preferred. In some embodiments, certain muscle-specific promoters express more highly in certain muscles than others, e.g. cardiac muscle, skeletal muscle, SEQ ID NO: 18 (SP0514) and SEQ ID NO: 23 (SP0519) are particularly preferred.

In some embodiments, synthetic muscle-specific promoters comprising or consisting of SEQ ID NO: 4 (SP0500), or functional variants thereof, are particularly preferred. SP0500 is primarily active in certain muscles such as cardiac muscle with activity observed in skeletal muscle; SP0500 is more highly expressed in cardiac muscle as compared to skeletal muscle, see, e.g., FIGS. 5, 6 and 12. As such, SP0500 is a strong cardiac muscle-specific promoter and is less than 270 nucleotides long.

In some embodiments, synthetic muscle-specific promoters comprising or consisting of SEQ ID NO: 27 (SP0522), or functional variants thereof, are particularly preferred. SP0522 is primarily active in cardiac muscle with activity observed in skeletal muscle; SP0522 is more highly expressed in cardiac muscle as compared to skeletal muscle, see, e.g., FIGS. 5, 6 and 17. As such, SP0522 is a strong cardiac muscle-specific promoter and is less than 240 nucleotides long.

In some embodiments, synthetic muscle-specific promoters comprising or consisting of SEQ ID NO: 29 (SP0524), or functional variants thereof, are particularly preferred. SP0524 is active in cardiac muscle and skeletal muscle; SP0524 exhibits comparable expression levels in cardiac muscle and skeletal muscle, see, e.g., FIGS. 5, 6 and 18. As such, SP0524 is a muscle-specific promoter with length of less than 250 nucleotides. In some embodiments, synthetic muscle-specific promoters comprising or consisting of SEQ ID NO: 22 (SP0518), or functional variants thereof, are preferred. SP0518 has activity in skeletal muscle and with activity observed in cardiac muscle.

In some embodiments, synthetic muscle-specific promoters comprising or consisting of SEQ ID NO: 11 (SP0507), or functional variants thereof, are preferred. SP0507 has activity in skeletal muscle and cardiac muscle.

In some embodiments, synthetic muscle-specific promoters comprising or consisting of SEQ ID NO: 18 (SP0514), or functional variants thereof, are preferred. SP0514 has activity in cardiac muscle with activity observed in skeletal muscle; SP0514 is more highly expressed in cardiac muscle as compared to skeletal muscle, see, e.g., FIGS. 5, 6 and 14.

In some embodiments, synthetic muscle-specific promoters comprising or consisting of SEQ ID NO: 23 (SP0519), or functional variants thereof, are preferred. SP0519 has activity in skeletal muscle with activity observed in cardiac muscle; SP0519 has increased expression in some skeletal muscle types as compared to cardiac muscle, see, e.g., FIGS. 5, 6 and 16.

In some embodiments, synthetic muscle-specific promoters comprising or consisting of SEQ ID NO: 4 (SP0500) or SEQ ID NO: 27 (SP0522), or functional variants thereof, and are particularly preferred. SP0500 and SP0522 are primarily active in cardiac muscle and have activity in skeletal muscle.

In some embodiments, synthetic muscle-specific promoters comprising or consisting of SEQ ID NO: 4 (SP0500), SEQ ID NO: 27 (SP0522) and SEQ ID NO: 29 (SP0524), or functional variants thereof, are particularly preferred.

In some embodiments, synthetic muscle-specific promoters comprising or consisting of SEQ ID NO: 4 (SP0500), SEQ ID NO: 27 (SP0522), SEQ ID NO: 22 (SP0518) and SEQ ID NO: 29 (SP0524), or functional variants thereof, are particularly preferred.

In some embodiments, synthetic muscle-specific promoters comprising or consisting of SEQ ID NO: 4 (SP0500), SEQ ID NO: 27 (SP0522), SEQ ID NO: 22 (SP0518), SEQ ID NO: 29 (SP0524), SEQ ID NO: 11 (SP0507), SEQ ID NO: 18 (SP0514) and SEQ ID NO: 23 (SP0519) or functional variants thereof, are particularly preferred.

In some embodiments, synthetic muscle-specific promoters comprising or consisting of SEQ ID NO: 66 (SP0321), or functional variant thereof, may be particularly preferred. SP0321 is expected to be active in muscle. SP0321 is also expected to be active in lung. As such, SP0321 is expected to be a muscle-specific and lung-specific promoter.

In some embodiments, the synthetic muscle-specific synthetic promoter according to the present invention has higher activity than CK8, CK7 or CMV in the diaphragm. In some embodiments, the synthetic muscle-specific synthetic promoter according to the present invention has higher activity than CK8, CK7 or CMV in the TA. In some embodiments, the synthetic muscle-specific synthetic promoter according to the present invention has higher activity than CK8, CK7 or CMV in the heart. In some embodiments, the synthetic muscle-specific synthetic promoter according to the present invention has higher activity than CK8, CK7 or CMV in the quadriceps. In some embodiments, the synthetic muscle-specific synthetic promoter according to the present invention has higher activity than CK8, CK7 or CMV in the soleus. In some embodiments, the synthetic muscle-specific synthetic promoter according to the present invention has lower activity than CK8, CK7 or CMV in the liver. In some embodiments, the higher activity is at least 5, 10, 15, 20, 25, 30, 35, 40, 45, 50, 55, 60, 65, 70, 75, 80, 85, 90, 95, 99%, or more, or at least 1×, 2×, 3×, 4×, 5×, 10×, 20×, 30×, 40×, 50×, 100×, 500×, 1000×, or more as compared to a control, e.g., CK8, CK7 or CMV, Examplary methodology to test the activity of a synthetic promoter can be found in Examples 2 and 3.

In some embodiments, the synthetic muscle-specific promoter according to the present invention is also active in other tissues or cells. In some embodiments, the synthetic muscle-specific promoter according to the present invention is also active in one or more of the following group of tissues or cells: CNS, liver, kidney, spleen, lung and duodenum.

In some embodiments, the synthetic muscle-specific promoter according to the present invention comprises one of the combinations of CREs, or functional variants thereof, operably linked to a promoter element, or functional variant thereof, as set out in Table 5 below.

In any of the combinations of CREs, or functional variants thereof, disclosed herein, the recited CREs may be present in any order. In some preferred embodiments, the CREs are present in the recited order (i.e. in an upstream to downstream order, with reference to their position with respect to an operably linked promoter element or gene). In any of the combinations of CREs, or functional variants thereof, disclosed herein, some or all of the recited CREs may suitably be positioned adjacent to one other in the CRM (i.e. without any intervening CREs or other regulatory elements). The CREs may be contiguous or non-contiguous (i.e. they can be positioned immediately adjacent to one another or they can be separated by a spacer or other sequence). In some embodiments, it is preferred that some or all of the CREs are contiguous. In some preferred embodiments, the CREs, or the functional variants thereof, are provided in the recited order and are adjacent to one another. For example, the synthetic muscle-specific CRM may comprise CRE0077 immediately upstream of CRE0075, and so forth. In some embodiments, the promoter element lies downstream of the CREs and is typically adjacent to the proximal CRE. The promoter element may be contiguous with the adjacent CRE, or it can be separated by a spacer.

In a further aspect of the invention, there is provided an expression cassette comprising a synthetic muscle-specific promoter or skeletal muscle-specific promoter of any aspect of the present invention operably linked to a sequence encoding an expression product, suitably a gene, e.g. a transgene. In some embodiments the expression product is a therapeutic expression product.

The therapeutic expression product may be a therapeutic expression product useful in the treatment of any condition where expression in muscle may be of use, e.g. for treatment of a muscle condition or the treatment of a condition where secretion of the therapeutic expression product from the muscle may be desirable. The therapeutic expression product may be a therapeutic expression product useful in the treatment of a cardiovascular condition or heart disease and disorders such as heart failure or CHF. The therapeutic expression product may be a therapeutic expression product useful in the treatment of a skeletal muscle condition, disease or disorder such as any type of muscular dystrophy.

The sequence encoding the therapeutic expression product may be one or more genes which replace the function of one or more genes which are impaired or not functional in an autosomal, X-linked or Y-linked, dominant or recessive, disease. The sequence encoding the therapeutic expression product may be one or more genes encoding a replacement wild wild-type counterpart of a protein which is impaired or not functional in an autosomal, X-linked or Y-linked, dominant or recessive, disease. The sequence encoding the therapeutic expression product may be a gene encoding a replacement wild-type counterpart of a protein which is impaired or not functional function in an autosomal recessive disease.

The sequence encoding the therapeutic expression product may be a gene found in the human genome or a synthetic gene. Suitably, the therapeutic expression product may be a gene found in the human genome. The sequence encoding the therapeutic expression product may be the dysferlin gene (DYSF gene) and the therapeutic expression product may be the dysferlin protein. Mutations in the DYSF gene which impair the function of the dysferlin protein lead to dysferlinopathies. Examples of dysferlinopathies caused by mutations in the DYSF gene include Miyoshi myopathy, Limb Girdle muscular dystrophy type 2B and Distal Myopathy. An expression cassette comprising a synthetic muscle-specific promoter or skeletal muscle-specific promoter of any aspect of the present invention operably linked to a sequence encoding a therapeutic expression product, wherein the sequence encoding the therapeutic expression product is the DYSF gene may be useful in the treatment of dysferlinopathies such as Miyoshi myopathy, Limb Girdle muscular dystrophy type 2B and Distal Myopathy.

The sequence encoding the therapeutic expression product may be the dystrophin gene (DMD gene) and the therapeutic expression product may be the dystrophin protein. Mutations in the DMD gene which impair the function of the dystrophin protein lead to Becker muscular dystrophy or Duchenne muscular dystrophy which are X linked recessive muscular dystrophy disorders characterised by muscle weakness. An expression cassette comprising a synthetic muscle-specific promoter or skeletal muscle-specific promoter of any aspect of the present invention operably linked to a sequence encoding a therapeutic expression product, wherein the sequence encoding the therapeutic expression product is the DMD gene may be useful in the treatment of Becker muscular dystrophy or Duchenne muscular dystrophy.

The DMD gene is the largest known gene in human (approximately 2.4 million base pairs). Therefore, the full-length DMD gene is too large to be packaged into some viral vectors with limited capacity (payload) such as AAV vectors. Shorter versions of the DMD gene (called mini-dystrophins) are being used in order to combat this problem. Nonetheless, even the mini-dystrophins are fairly large (e.g. 3.5-4 KB) which still makes AAV packaging difficult. Therefore, a synthetic muscle-specific promoter of short length (e.g. less than 400 nucleotides in length, less than 350 nucleotides in length, preferably less than 300 nucleotides in length, yet more preferably less than 290 nucleotides in length, most preferably less than 280, 270, 260, 250, 240, 230, 220, 210, 200, 150, 100, 75, 70, 68 in length) may be particularly preferred in an expression cassette wherein the sequence encoding the therapeutic expression product is the DMD gene or a mini version of the DMD gene (mini-dystrophin).

In some embodiments, the expression cassette comprises a synthetic muscle-specific promoter or skeletal muscle-specific promoter of any aspect of the present invention operably linked to a short version of the DMD gene (mini-dystrophin). In some embodiments, the expression cassette comprises a synthetic muscle-specific promoter according to SEQ ID NO: 29 (SP0524), or functional variant thereof, operably linked to a short version of the DMD gene (mini-dystrophin).

The sequence encoding the therapeutic expression product may be miRNA or snRNA targeting and reducing the expression of the endogenous DUX4 gene. Gain of function mutation in the DUX4 gene leads to loss of repression of the DUX4 transcription factor which in turn results in deleterious gene expression changes causing facioscapulohumeral muscular dystrophy (FSHD) which is an autosomal dominant muscular dystrophy disorder characterised by progressive muscle impairment. An expression cassette comprising a synthetic muscle-specific promoter or skeletal muscle-specific promoter of any aspect of the present invention operably linked to a sequence encoding a therapeutic expression product, wherein the sequence encoding the therapeutic expression product is miRNA or snRNA targeting and reducing the expression of the endogenous DUX4 gene may be useful in the treatment of FSHD.

The sequence encoding the therapeutic expression product may be miRNA or snRNA targeting and reducing the expression of the endogenous myotonin-protein kinase gene (DMPK gene). Mutations in the DMPK gene lead to Myotonic dystrophy type 1 (DM1) which is an autosomal dominant muscular dystrophy disorder characterised by impaired muscle function. An expression cassette comprising a synthetic muscle-specific promoter or skeletal muscle-specific promoter of any aspect of the present invention operably linked to a sequence encoding a therapeutic expression product, wherein the sequence encoding the therapeutic expression product is the miRNA or snRNA targeting and reducing the expression of the endogenous DMPK gene may be useful in the treatment of DM1. In some embodiments, the expression cassette may comprise one or more synthetic muscle-specific promoters or skeletal muscle-specific promoters of any aspect of the present invention operably linked to miRNA or snRNA targeting and reducing the expression of the endogenous DMPK gene and a wild type replacement DMPK gene. In some embodiments, the expression cassette may comprise one or more synthetic muscle-specific promoters or skeletal muscle-specific promoters of any aspect of the present invention operably linked to miRNA or snRNA targeting and reducing the expression of the endogenous DMPK gene and a wild type replacement MBNL1 gene. In some embodiments, the expression cassette may comprise one or more synthetic muscle-specific promoters or skeletal muscle-specific promoters of any aspect of the present invention operably linked to miRNA or snRNA targeting and reducing the expression of the endogenous DMPK gene, a wild type replacement DMPK gene and wild type replacement MBNL1 gene.

The sequence encoding the therapeutic expression product may be miRNA or snRNA targeting and reducing the expression of the endogenous cellular nucleic acid-binding protein gene (CNBP gene). Gain of function mutations in the CNBP gene lead to Myotonic dystrophy type 2 (DM2) which is an autosomal dominant muscular dystrophy disorder characterised by impaired muscle function. An expression cassette comprising a synthetic muscle-specific promoter or skeletal muscle-specific promoter of any aspect of the present invention operably linked to a sequence encoding a therapeutic expression product, wherein the sequence encoding the therapeutic expression product is the miRNA or snRNA targeting and reducing the expression of the endogenous CNBP gene may be useful in the treatment of DM2.

The sequence encoding the therapeutic expression product may be miRNA or snRNA targeting and reducing the expression of the endogenous polyadenylate-binding protein 2 gene (PABPN1 gene). Gain of function mutations in the CNBP gene lead to Oculopharyngeal muscular dystrophy which is an autosomal dominant or autosomal recessive muscular dystrophy disorder. An expression cassette comprising a synthetic muscle-specific promoter or skeletal muscle-specific promoter of any aspect of the present invention operably linked to a sequence encoding a therapeutic expression product, wherein the sequence encoding the therapeutic expression product is miRNA or snRNA targeting and reducing the expression of the endogenous PABPN1 gene may be useful in the treatment of Oculopharyngeal muscular dystrophy. In some embodiments, the expression cassette may comprise one or more synthetic muscle-specific promoters or skeletal muscle-specific promoters of any aspect of the present invention operably linked to miRNA or snRNA targeting and reducing the expression of the endogenous PABPN1 gene and a synthetic replacement PABN1 gene. Suitably, the synthetic replacement has been modified or codon optimised so as not to be a target of the miRNA or snRNA reducing the expression of the endogenous PABPN1 gene.

The sequence encoding the therapeutic expression product may be the C1C-1 ion channel gene (CLCN1 gene) and the therapeutic expression product may be the C1C-1 ion channel. Mutations in the CLCN1 gene lead to Myotonia congenita which is an autosomal dominant or autosomal recessive channelopathy affecting skeletal muscle. An expression cassette comprising a synthetic muscle-specific promoter or skeletal muscle-specific promoter of any aspect of the present invention operably linked to a sequence encoding a therapeutic expression product, wherein the sequence encoding the therapeutic expression product is the CLCN1 gene may be useful in the treatment of Myotonia congenita.

The sequence encoding the therapeutic expression product may be the voltage-gated sodium channel Nav 1.4 gene (SCN4A gene) and the therapeutic expression product may be the voltage-gated sodium channel Nav 1.4. Mutations in the SCN4A gene lead to Paramyotonia congenita, Potassium-aggravated myotonia, Hyperkalemia periodic paralysis and Hypokalemic periodic paralysis. An expression cassette comprising a synthetic muscle-specific promoter or skeletal muscle-specific promoter of any aspect of the present invention operably linked to a sequence encoding a therapeutic expression product, wherein the sequence encoding the therapeutic expression product is the SCN4A gene may be useful in the treatment of Paramyotonia congenita, Potassium-aggravated myotonia, Hyperkalemia periodic paralysis and Hypokalemic periodic paralysis.

The sequence encoding the therapeutic expression product may be the SEPN1 or RYR1 genes. Mutations in the SEPN1 or RYR1 genes lead to Multi/minicore myopathy. An expression cassette comprising a synthetic muscle-specific promoter or skeletal muscle-specific promoter of any aspect of the present invention operably linked to a sequence encoding a therapeutic expression product, wherein the sequence encoding the therapeutic expression product is the SEPN1 gene or the RYR1 gene may be useful in the treatment of Multi/minicore myopathy.

Suitably, the sequence encoding the therapeutic expression product may be a synthetic gene. The synthetic gene may be a gene found in the human genome which has been shortened, for example shortened to allow packaging in an AAV vector. The synthetic gene may be a gene found in the human genome which has been optimised for quicker translation, for example codon optimised or alternatively an artificial construct. Codon optimisation is well understood by the person skilled in the art and refers to adjusting synonymous codons to accommodate the codon bias of the host organism in order to improve gene expression and increase translational efficiency. The synthetic gene may be a gene found in the human genome which has been modified so that its expression protein has specific intracellular localisation,
for example a gene which has been modified to include a nuclear localisation signal such that the protein with this signal is imported in the cell nucleus by nuclear transport. The synthetic gene may be a gene found in the human genome which has been modified so that it is secreted more efficiently, for example a gene which has been modified to include a secretory signal which such that a protein with this signal is targeted for translocation across the endoplasmic reticulum membrane and secretion into the environment. The synthetic gene in some embodiments may be a gene found in the human genome which has been modified so that its expression product is less immunogenic, for example by removal of B cell epitopes. The synthetic gene may be a gene found in the human genome which has been modified so that its function is increased or decreased. The synthetic gene may be a gene found in the human genome which has been modified so that it is not silenced by specific miRNA or snRNA. Any gene found in the human genome may be modified by one or more of the modifications described above resulting in a synthetic gene. In some embodiments, the gene which has been modified is any one of the genes listed in any of the aspects herein.

The sequence encoding the therapeutic expression product may be a synthetic dysferlin gene (DYSF gene) and the therapeutic expression product may be a synthetic dysferlin protein. The sequence encoding the therapeutic expression product may be a synthetic dysferlin gene (DYSF gene) which has been shortened. The sequence encoding the therapeutic expression product may be a synthetic dysferlin gene (DYSF gene) which has been shortened to allow packaging in an AAV vector. The sequence encoding the therapeutic expression product may be a synthetic dysferlin gene (DYSF gene) which has been shortened to less than 6 kb, less than 5.5 kb, less than 5 kb, less than 4.5 kb, less than 4 kb, less than 3.5 kb or less than 3 kb. The sequence encoding the therapeutic expression product may be a synthetic dysferlin-like molecule shortened to be amendable to single AAV vector packaging. The sequence encoding the therapeutic expression product may be Nano-Dysferin as detailed in table 1 and FIG. 1A of (Llanga et al., 2017).

In some preferred embodiments, the expression cassette comprises a muscle-specific promoter according to any aspect of the present invention operably linked to Nano-Dysferin as detailed in table 1 and FIG. 1A of (Llanga et al., 2017).

The therapeutic expression product may be a modulator of phosphatase activity, e.g., type 1 phosphatase activity. The modulator may be a protein that inhibits phosphatase activity, e.g., type 1 phosphatase activity. The modulator may be a nucleic acid that increases expression of an endogenous nucleic acid that encodes a protein that inhibits phosphatase activity such as a transcription factor. The modulator may be a regulatory sequence that integrates in or near the endogenous nucleic acid that encodes a protein that inhibits phosphatase activity. The modulator may be a nucleic acid that can provide a nucleic acid modulator of gene expression such as a siRNA.

The therapeutic expression product may be inhibitor of protein phosphate 1 (PP1) e.g., a I-1 polypeptide. Type 1 phosphatases include, but are not limited to, PP1cα, PP1cβ, PP1cδ and PP1cγ. The phosphatase inhibitor-1 (or “I-1”) protein is an endogenous inhibitor of type 1 phosphatase. Increasing I-1 levels or activity can restore β-adrenergic responsiveness in failing human cardiomyocytes. Suitably, the I-1 protein may be constitutively active such as a I-1 protein where threonine 35 is replaced with glutamic acid instead of aspartic acid. The therapeutic expression product may be any one or more of the inhibitors selected from: phosphatase inhibitor 2 (PP2); okadaic acid or caliculin; and nippl which is an endogenous nuclear inhibitor of protein phosphatase 1.

The therapeutic expression product may be any protein that modulates cardiac activity such as a phosphatase type 1 inhibitor, e.g., I-1 or a sacroplasmic reticulum Ca2+ ATPase (SERCA), e.g., SERCA1 (e.g., 1a or 1b), SERCA2 (e.g., 2a or 2b), or SERCA3.

The therapeutic expression product may be nucleic acid sequence encoding a mutant form of phosphatase inhibitor-1 protein, wherein the mutant form comprises at least one amino acid at a position that is a PKC-α phosphorylation site in the wild type, wherein the at least one amino acid is constitutively unphosphorylated or mimics an unphosphorylated state in the mutant form. The therapeutic expression product may be adenylyl cyclase 6 (AC6, also referred to as adnenylyl cyclase VI), S100A1, β-adrenergic receptor kinase-ct (βARKct), sarco/endoplasmic reticulum (SR) Ca-ATPase (SERCA2a), IL-18, VEGF, VEGF activators, urocortins, and B-cell lymphoma 2 (Bcl2)-associated anthanogene-3 (BAG3).

The therapeutic expression product may be an inhibitor of a cytokine such as an IL-18 inhibitor. The therapeutic expression product may encode a beta-adrenergic signalling protein (beta-ASPs) (including beta-adrenergic receptors (beta-Ars), G-protein receptor kinase inhibitors (GRK inhibitors) and adenylylcyclases (Acs)) to enhance cardiac function.

The therapeutic expression product may be an angiogenic protein. Angiogenic proteins promote development and differentiation of blood vessels. Examples of angiogenic proteins include members of the fibroblast growth factor (FGF) family such as aFGF (FGF-1), bFGF (FGF-2), FGF-4 (also known as “hst/KS3”), FGF-5 and FGF-6, the vascular endothelial growth factor (VEGF) family, the platelet-derived growth factor (PDGF) family, the insulin-like growth factor (IGF) family, and others.

In a further aspect, there is provided a vector comprising a synthetic muscle-specific promoter, skeletal muscle-specific promoter or an expression cassette according to the present invention. In some embodiments the vector is an expression vector. In some embodiments the vector is a viral vector. In some embodiments the vector is a gene therapy vector, suitably an AAV vector, an adenoviral vector, a retroviral vector or a lentiviral vector. AAV vectors are of particular interest. AAV vectors may be selected from the group consisting of AAV2, AAV6, AAV8, AAV9, BNP116, rh10, AAV2.5, AAV2i8, AAVDJ8 and AAV2G9, or derivatives thereof. AAV serotype 9 (AAV9) has been noted to achieve efficient transduction in cardiac and skeletal muscle, and thus AAV9 and derivatives thereof represent one non-limiting example of a suitable AAV vector. In some embodiments, the rAAV vector is an AAV3b serotype, including, but not limited to, an AAV3b265D virion, an AAV3b265D549A virion, an AAV3b549A virion, an AAV3bQ263Y virion, or an AAV3bSASTG virion (i.e., a virion comprising an AAV3b capsid comprising Q263A/T265 mutations). In some embodiments, the virion can be rational haploid, or a chimeric or any mutant, such as capsids can be tailored for increased update at a desired location, e.g., the heart or skeletal muscle. Other capsids can include capsids from any of the known AAV serotypes, including AAV1, AAV3, AAV4, AAV5, AAV7, AAV10, etc. In some preferred embodiments, the AAV vector is AAV2i8. In some embodiments, the AAV vector is AAV9.

The vector according to the present invention may be an AAV vector comprising a nucleic acid encoding a therapeutic expression product for treatment of muscular dystrophy, wherein the nucleic acid is operatively linked to a skeletal muscle-specific promoter or a muscle-specific promoter.

The vector according to the present invention may be an AAV vector comprising a nucleic acid encoding a therapeutic expression product for treatment of heart failure, wherein the nucleic acid is operatively linked to a muscle-specific promoter, which may be a cardiac muscle specific promoter.

In a further aspect, there is provided a virion (viral particle) comprising a vector, suitably a viral vector, according to the present invention. In some embodiments the virion is an AAV virion. Suitable virions are described above.

In a further aspect, there is provided a pharmaceutical composition comprising a synthetic muscle-specific promoter, synthetic skeletal muscle-specific promoter, expression cassette, vector or virion according to the present invention.

In a further aspect, there is provided a synthetic muscle-specific promoter, synthetic skeletal muscle-specific promoter, expression cassette, vector, virion or pharmaceutical composition according to the present invention for use in therapy, i.e. the prevention or treatment of a medical condition or disease. Suitably, for use in therapy of subject in need thereof. Suitably the condition or disease is associated with aberrant gene expression, optionally aberrant gene expression in muscle cells (myocytes) or tissue. Suitably the condition or disease is associated with aberrant gene expression in skeletal muscle or tissue. Suitably the condition or disease is associated with aberrant gene expression, in cardiac muscle cells or heart tissue. Suitably, there is provided a synthetic muscle-specific promoter, synthetic skeletal muscle-specific promoter, expression cassette, vector, virion or pharmaceutical composition according to the present invention for use in expression of a therapeutic expression product in skeletal and/or cardiac muscle.

In one embodiment, the disease may be skeletal muscle disease or disorder. In one embodiment, the disease may be muscular dystrophy such as Becker muscular dystrophy, Congenital muscular dystrophy, Duchenne muscular dystrophy, Distal muscular dystrophy, Emery-Dreifuss muscular dystrophy, Facioscapulohumeral muscular dystrophy, Limb Girdle muscular dystrophy, Myotonic muscular dystrophy type 1, Myotonic muscular dystrophy type 2 or Oculopharyngeal muscular dystrophy. In one embodiment, the disease may be myotonia such as Myotonia congenita, Paramyotonia congenita, Potassium-aggravated myotonia, Hyperkalemia periodic paralysis or Hypokalemic periodic paralysis. In one embodiment, the disease may be congenital myopathy selected from nemaline myopathy, Multi/minicore myopathy and Centronuclear myopathy. In one embodiment, the disease may be selected from mictochondrial myopathies, periodic paralysis, inflammatory myopathies, metabolic myopathies, Brody myopathy and Hereditary inclusion body myopathy. Suitably, the use is for gene therapy, preferably for use in treatment of a disease involving aberrant gene expression. Suitably the gene therapy involves expression of a therapeutic expression product in muscle cells or tissue, suitably in skeletal muscle cells or skeletal tissue and/or cardiac muscle cells or heart tissue.

In one embodiment, the disease may be cardiovascular condition or heart disease and disorders. In one embodiment, the disease may be heart failure such as congestive heart failure. In one embodiment, the disease may be selected from ischemia, arrhythmia, myocardial infarction (MI), abnormal heart contractility, non-ischemic cardiomyopathy, peripheral arterial occlusive disease, and abnormal Ca²⁺ metabolism, and combinations thereof. In some embodiments, the disease may be selected from the group of: congestive heart failure, cardiomyopathy, myocardial infarction, tissue ischemia, cardiac ischemia, vascular disease, acquired heart disease, congenital heart disease, atherosclerosis, dysfunctional conduction systems, dysfunctional coronary arteries, pulmonary heart hypertension. In some embodiments, the disease may be selected from congestive heart failure, coronary artery disease, myocardial infarction, myocardial ischemia, atherosclerosis, cardiomyopathy, idiopathic cardiomyopathy, cardiac arrhythmias, Danon disease, muscular dystrophy, muscle mass abnormality, muscle degeneration, infective myocarditis, drug- or toxin-induced muscle abnormalities, hypersensitivity myocarditis, an autoimmune endocarditis and congenital heart disease. Suitably the use is for gene therapy, preferably for use in treatment of a disease involving aberrant gene expression. Suitably the gene therapy involves expression of a therapeutic expression product in muscle cells or tissue, suitably in cardiac muscle cells or heart tissue and/or suitably in skeletal muscle cells or skeletal tissue.

In some embodiments, the methods and compositions disclosed herein can be used to treat a subject with cardiomyopathy, where the subject with cardiomyopathy has heart failure. In such an embodiment, the subject with heart failure has a classification that is equivalent to class III or above in the New York Heart Association (NYHA) classification system. In some embodiments, the subject with heart failure has a cardiovascular disease or heart disease is selected from any of: left ventricular remodeling, peripheral arterial occlusive disease (PAOD), dilated cardiomyopathy (DCM) including idiopathic dilated cardiomyopathy (IDCM), coronary artery disease, ischemia, arrhythmia, myocardial infarction (MI), abnormal heart contractility, acute (decompensated) heart failure (AHF), abnormal Ca2+ metabolism, myocardial ischemia, atherosclerosis, cardiomyopathy, idiopathic cardiomyopathy, genetic disorder induced cardiomyopathy, cardiac arrhythmias, Danon disease, muscular dystrophy, muscle mass abnormality, muscle degeneration, infective myocarditis, drug- or toxin-induced muscle abnormalities, hypersensitivity myocarditis, an autoimmune endocarditis and congenital heart disease and pulmonary heart hypertension.

In some embodiments, the methods and compositions disclosed herein can be used to treat a subject with cardiomyopathy, wherein the subject with cardiomyopathy has non-ischemic heart failure and/or non-ischemic cardiomyopathy, including but not limited to, acquired cardiomyopathy, cardiomyopathy acquired as a result of an infection, or toxin, etc., or a congenital cardiomyopathy or a genetic disorder with a cardiac manifestation. In some embodiments, a subject with a congenital cardiomyopathy or a genetic disorder with a cardiac manifestation has a disease or disorder selected from the group consisting of: Arrhythmogenic right ventricular cardiomyopathy, Atrial myxoma, familial, Atrial septal defect ostium primum, Atrial septal defect sinus venosus, Barth syndrome, muscular dystrophy, Buerger disease, Cardioencephalomyopathy, Chromosome 1p36 deletion syndrome, Congenital generalized lipodystrophy type 4, Congenital heart block, Dilated cardiomyopathy, Duchenne muscular dystrophy (DMD), Fabry disease, Familial atrial fibrillation, Familial dilated cardiomyopathy, Familial hypertrophic cardiomyopathy, Familial progressive cardiac conduction defect, Familial thoracic aortic aneurysm and aortic dissection, Fibromuscular dysplasia, Friedreich ataxia, Gaucher disease, Glycogen storage disease (types 2, 3 or 4), His bundle tachycardia, Hurler syndrome, Hypoplastic left heart syndrome, Infantile histiocytoid cardiomyopathy, Intracranial arteriovenous malformation, Isobutyryl-CoA dehydrogenase deficiency, Kallikrein hypertension, Kawasaki disease, Kearns-Sayre syndrome, Left ventricular noncompaction, Limb-girdle muscular dystrophy (types 1B, 2E, 2F, 2M, 2C, 2D), Limited systemic sclerosis, Long QT syndrome 1, Lymphedema and cerebral arteriovenous anomaly, Lymphocytic vasculitis, Microcephaly-cardiomyopathy; Mitochondrial encephalomyopathy lactic acidosis and stroke-like episodes, Mitochondrial trifunctional protein deficiency, Myotonic dystrophy type 1, Neonatal stroke, Noonan syndromes 1-, 2-, 3-, 4-, 5- and 6, Peripartum cardiomyopathy, Peters plus syndrome, PGM1-CDG, PHACE syndrome, Phospholamban Arg 14 Deletion, Postural orthostatic tachycardia syndrome, Primary carnitine deficiency, Progressive familial heart block (types 1A, 1B and 2), Pseudohypoaldosteronism type 2, Pulmonary arterial hypertension, Pulmonary atresia with intact ventricular septum, Pulmonary atresia with ventricular septal defect, Pulmonary valve stenosis, Pulmonary vein stenosis, Pulmonic stenosis, Renoprival hypertension, Retinal arterial macroaneurysm with supravalvular pulmonic stenosis, Right ventricle hypoplasia, Sarcoidosis, Sengers syndrome, Situs inversus, Sudden Arrhythmia Death Syndrome, Supravalvular aortic stenosis, Swyer syndrome, TANGO2-Related Metabolic Encephalopathy and Arrhythmias, TARP syndrome, Tetralogy of Fallot, Timothy syndrome, Tricuspid atresia, Vici syndrome, VLCAD deficiency and Williams syndrome.

In some embodiments, the methods and compositions disclosed herein can be used to treat a subject with cardiomyopathy, wherein the subject with cardiomyopathy has an ischemic cardiomyopathy.

Suitably, the subject in need of therapy will display symptoms characteristic of a skeletal muscle condition, e.g., muscular dystrophy as discussed above. The medical use typically comprises ameliorating the symptoms displayed by the subject in need thereof, by expressing the therapeutic amount of the therapeutic product. In some embodiments, the expression cassette comprises a gene encoding dysferlin or synthetic dysferlin, operably linked to a skeletal muscle-specific promoter or muscle-specific promoter. The therapy suitably comprises expressing a therapeutic amount of dysferlin or synthetic dysferlin in the skeletal muscle tissue of said subject. Suitably, expressing a therapeutic amount of dysferlin or synthetic dysferlin in skeletal muscle tissue reduces the symptoms of dysferlinopahy in a subject. Suitably, expressing a therapeutic amount of dysferlin or synthetic dysferlin in skeletal muscle tissue may attenuate weakness and wasting of skeletal muscles.

Suitably, the subject in need of therapy will display symptoms characteristic of a cardiovascular condition, e.g., heart disease or heart failure as discussed above. The medical use typically comprises ameliorating the symptoms displayed by the subject in need thereof, by expressing the therapeutic amount of the therapeutic product. In some embodiments, the expression cassette comprises a gene encoding an inhibitor of the PP1, operably linked to a muscle-specific promoter. The therapy suitably comprises expressing a therapeutic amount of the inhibitor of PP1 in the heart tissue of said subject. Suitably, expressing a therapeutic amount of the inhibitor of PP1 in the heart tissue reduces the symptoms of heart failure or a heart disorder of a subject. Suitably, expressing a therapeutic amount of the inhibitor of PP1 in the heart tissue may attenuate cardiac remodelling, improve exercise capacity, or improve cardiac contractility. Suitably, expressing a therapeutic amount of the inhibitor of PP1 in the heart tissue may result in myocyte shortening, lowering of the time constant for relaxation, and accelerating calcium signal decay, improving the end-systolic pressure dimension relationship and combinations thereof.

In a further aspect, there is provided a cell comprising a synthetic muscle-specific promoter, synthetic skeletal muscle-specific promoter, expression cassette, vector, or virion of the present invention. In some embodiments the cell is a eukaryotic cell, optionally a mammalian cell, optionally a human cell. Suitably the cell can be a muscle cell, optionally wherein the cell is a human muscle cell. Suitably a human skeletal muscle cell or human cardiac muscle cell. The synthetic muscle-specific promoter, synthetic skeletal muscle-specific promoter, expression cassette, vector, or virion of the present invention can be episomal or can be in the genome of the cell.

In a further aspect, there is provided a synthetic muscle-specific CRE, CRM, synthetic muscle-specific promoter, synthetic skeletal muscle-specific promoter, expression cassette, vector, virion or pharmaceutical composition as described herein for use in the manufacture of a pharmaceutical composition for the treatment of a medical condition or disease.

In a further aspect, there is provided a method for producing an expression product, the method comprising providing a synthetic muscle-specific or skeletal muscle-specific expression cassette of the present invention in a muscle cell and expressing the gene present in the expression cassette. The method can be in vitro or ex vivo, or it can be in vivo. In some embodiments the method is bioprocessing method. In one embodiment, the muscle cell is a skeletal muscle cell. In one embodiment, the muscle cell is a cardiac muscle cell.

In a further aspect, there is provided a method of expressing a therapeutic transgene in a muscle cell, the method comprising introducing into the muscle cell a synthetic muscle-specific or skeletal-muscle specific expression cassette, vector or virion as described herein. In one embodiment, the muscle cell is a skeletal muscle cell. In one embodiment, the muscle cell is a cardiac muscle cell.

In a further aspect, there is provided a method of therapy of a subject, preferably a human, in need thereof, the method comprising:
administering to the subject an expression cassette, vector, virion or pharmaceutical composition as described herein, which comprises a sequence encoding a therapeutic product operably linked to a promoter according to the present invention; and
expressing a therapeutic amount of the therapeutic product in the muscle of said subject.

A therapeutic product is a product used to prevent, alleviate, cure or positively modify a physiological process or disease.

In one embodiment, the muscle is a skeletal muscle cell or tissue. In one embodiment, the muscle is a cardiac muscle cell or tissue. Suitably, the method of therapy of a subject comprises expression of the therapeutic amount of the therapeutic product in the skeletal and/or cardiac muscle.

In some embodiments the method comprises:
introducing into the muscle of the subject an expression cassette, vector, virion or pharmaceutical composition as described herein, which comprises a gene encoding a therapeutic product; and
expressing a therapeutic amount of the therapeutic product in the muscle of said subject.

In one embodiment, the muscle is a skeletal muscle cell or tissue. In one embodiment, the muscle is a cardiac muscle cell or tissue. Suitably, the method comprises expression of the therapeutic amount of the therapeutic product in the skeletal and/or cardiac muscle of said subject.

Suitably the method comprises administering a vector, virion or pharmaceutical composition as described herein to the subject. In some preferred embodiments the vector is a viral gene therapy vector, preferably an AAV vector.

In some embodiments, the method of therapy comprises administering to the subject a viral vector, which comprises a sequence encoding a therapeutic product operably linked to a promoter according to the present invention. In some preferred embodiments, the viral vector is administered to the subject by anterograde epicardial coronary artery infusion (AECAI). In some particularly preferred embodiments, the viral gene therapy vector is administered to the subject by anterograde epicardial coronary artery infusion (AECAI) through a percutaneous femoral access. In some embodiments, the subject has heart failure or congestive heart failure. In some embodiments, the method of therapy of the subject is for treatment of heart failure or congestive heart failure.

Further features and embodiments of the present invention will now be described under the following sections. Any feature or embodiment in any section may be combined with any other feature or embodiment, or with any aspect of the invention, in any workable combination.

In some embodiments, a synthetic muscle-specific promoter comprises two or more promoter elements. Synthetic promoters comprising two or more promoter elements are referred to herein as ‘tandem promoters’. CRE0138 which is herein designated as a CRE comprises the transcriptional start site of the TNNI2 gene and may be expected to perform similarly to a promoter element. Therefore, for example, SP0508 may be considered to be a tandem promoter as it comprises promoter elements CRE0138 and CRE0053. Similarly, SP0519 may be considered to be a tandem promoter as it comprises promoter elements CRE0138 and BG mp.

In some embodiments, a tandem promoter may comprise a promoter element directly upstream of another promoter element. In some embodiments, a tandem promoter may comprise one or more CREs upstream of one or each of the promoter elements. In some embodiments, a tandem promoter may comprise one or more CREs between the promoter elements. In some embodiments, any one of the synthetic muscle-specific promoters disclosed herein may be operably linked to a further promoter element. It will be appreciated that any other synthetic promoter disclosed herein may be further operably linked to any promoter element disclosed herein.

### BRIEF DESCRIPTION OF THE FIGURES

FIG. 1 shows the average activity of synthetic muscle-specific promoters according to some embodiments of this invention in H9C2 cell line differentiated into heart myotubes. The error bar is standard deviation. CBA and CK8 are control promoters. The y-axis is luciferase activity (Relative light units).

FIG. 2 shows the average activity of synthetic muscle-specific promoters according to some embodiments of this invention in H9C2 cell line differentiated into heart myotubes. The error bar is standard deviation. CBA and CK8 are control promoters. The average activity has been normalised relative to control promoter CBA. The y-axis is luciferase activity (Relative light units).

FIG. 3 shows the in vivo activity of synthetic muscle-specific promoters SP0500, SP0507, SP0514, SP0518, SP0519, SP0522 and SP0524 and the control promoters CK8, CMV, CK7 in the diaphragm. Saline control (background) was also included in the experiment. The saline control (background) was subtracted from the luciferase expression of each promoter and then this value was divided by vector copy number of each promoter in the diaphragm.

FIG. 4 shows the in vivo activity of synthetic muscle-specific promoters SP0500, SP0507, SP0514, SP0518, SP0519, SP0522 and SP0524 and the control promoters CK8, CMV, CK7 in the tibialis anterior (TA). Saline control (background) was also included in the experiment. The saline control (background) was subtracted from the luciferase expression of each promoter and then this value was divided by vector copy number of each promoter in the diaphragm.

FIG. 5 shows the in vivo activity of synthetic muscle-specific promoters SP0500, SP0507, SP0514, SP0518, SP0519, SP0522 and SP0524 and the control promoters CK8, CMV, CK7 in the heart. Saline control (background) was also included in the experiment. The saline control (background) was subtracted from the luciferase expression of each promoter and then this value was divided by vector copy number of each promoter in the diaphragm.

FIG. 6 shows the in vivo activity of synthetic muscle-specific promoters SP0500, SP0507, SP0514, SP0518, SP0519, SP0522 and SP0524 and the control promoters CK8, CMV, CK7 in the quadriceps. Saline control (background) was also included in the experiment. The saline control (background) was subtracted from the luciferase expression of each promoter and then this value was divided by vector copy number of each promoter in the diaphragm.

FIG. 7 shows the in vivo activity of synthetic muscle-specific promoters SP0500, SP0507, SP0514, SP0518, SP0519, SP0522 and SP0524, the control promoters CK8, CMV, CK7 and saline control in the soleus. No vector copy number was available in the soleus so this graph shows luciferase expression of each promoter.

FIG. 8 shows the in vivo activity of synthetic muscle-specific promoters SP0500, SP0507, SP0514, SP0518, SP0519, SP0522 and SP0524, the control promoters CK8, CMV, CK7 in the liver. The saline control (background) was subtracted from the luciferase expression of each promoter and then this value was divided by vector copy number of each promoter in the diaphragm.

FIG. 9 shows the in vivo activity of control promoter CK8 in the diaphragm, tibialis anterior (TA), quadriceps, heart and liver.

FIG. 10 shows the in vivo activity of control promoter CMV in the diaphragm, tibialis anterior (TA), quadriceps, heart and liver.

FIG. 11 shows the in vivo activity of control promoter CK7 in the diaphragm, tibialis anterior (TA), quadriceps, heart and liver.

FIG. 12 shows the in vivo activity of synthetic muscle-specific promoter SP0500 in the diaphragm, tibialis anterior (TA), quadriceps, heart and liver.

FIG. 13 shows the in vivo activity of synthetic muscle-specific promoter SP0507 in the diaphragm, tibialis anterior (TA), quadriceps, heart and liver.

FIG. 14 shows the in vivo activity of synthetic muscle-specific promoter SP0514 in the diaphragm, tibialis anterior (TA), quadriceps, heart and liver.

FIG. 15 shows the in vivo activity of synthetic muscle-specific promoter SP0518 in the diaphragm, tibialis anterior (TA), quadriceps, heart and liver.

FIG. 16 shows the in vivo activity of synthetic muscle-specific promoter SP0519 in the diaphragm, tibialis anterior (TA), quadriceps, heart and liver.

FIG. 17 shows the in vivo activity of synthetic muscle-specific promoter SP0522 in the diaphragm, tibialis anterior (TA), quadriceps, heart and liver.

FIG. 18 shows the in vivo activity of synthetic muscle-specific promoter SP0524 in the diaphragm, tibialis anterior (TA), quadriceps, heart and liver.

### DETAILED DESCRIPTION OF EMBODIMENTS OF THE INVENTION AND EXAMPLES

### CREs and Functional Variants Thereof

Disclosed herein are various CREs that can be used in the construction of muscle-specific promoters. These CREs are generally derived from genomic promoter and enhancer sequences, but they are used herein in contexts quite different from their native genomic environment. Generally, the CREs constitute small parts of much larger genomic regulatory domains, which control expression of the genes with which they are normally associated. It has been surprisingly found that these CREs, many of which are very small, can be isolated form their normal environment and retain muscle-specific regulatory activity when used to construct various synthetic promoters. This is surprising because the removal of a regulatory sequence from the complex and “three dimensional” natural context in the genome often results in a significant loss of activity, so there is no reason to expect a given CRE to retain the levels of activity observed once removed from their natural environment. Many combinations of these CREs have been tested and found to be highly effective at enhancing muscle-specific promoter activity when combined with minimal and proximal promoters. It should be noted that the sequences of the CREs of the present invention can be altered without causing a substantial loss of activity. Functional variants of the CREs can be prepared by modifying the sequence of the CREs, provided that modifications which are significantly detrimental to activity of the CRE are avoided. In view of the information provided in the present disclosure, modification of CREs to provide functional variants is straightforward. Moreover, the present disclosure provides methodologies for simply assessing the functionality of any given CRE variant. Functional variant examples for are discussed below.

CRE0145 is a functional variant of CRE0050 and vice versa as CRE0145 is a shorter version of CRE0050.

The relatively small size of certain CREs according to the present invention is advantageous because it allows for the CREs, more specifically promoters containing them, to be provided in vectors while taking up the minimal amount of the payload of the vector. This is particularly important when a CRE is used in a vector with limited capacity, such as an AAV-based vector.

CREs of the present invention comprise certain muscle-specific TFBS. It is generally desired that in functional variants of the CREs these muscle-specific TFBS remain functional. The skilled person is well aware that TFBS sequences can vary yet retain functionality. In view of this, the sequence for a TFBS is typically illustrated by a consensus sequence from which some degree of variation is typically present. Further information about the variation that occurs in a TFBS can be illustrated using a positional weight matrix (PWM), which represents the frequency with which a given nucleotide is typically found at a given location in the consensus sequence. Details of TF consensus sequences and associated positional weight matrices can be found in, for example, the Jaspar or Transfac databases http://jaspar.genereg.net/and http://gene-regulation.com/pub/databases.html). This information allows the skilled person to modify the sequence in any given TFBS of a CRE in a manner which retains, and in some cases even increases, CRE functionality. In view of this the skilled person has ample guidance on how the TFBS for any given TF can be modified, while maintaining ability to bind the desired TF; the Jaspar system will, for example, score a putative TFBS based on its similarity to a given PWM. Furthermore, CREs can be scanned against all PWM from JASPAR database to identify/analyse all TFBS. The skilled person can of course find additional guidance in the literature, and, moreover, routine experimentation can be used to confirm TF binding to a putative TFBS in any variant CRE. It will be apparent that significant sequence modification in a CRE, even within TFBS in a CRE, can be made while retaining function.

### Synthetic Muscle-Specific CRMs and Functional Variants Thereof

Various synthetic muscle-specific CRMs are disclosed herein that can be used in the constructions of synthetic muscle-specific promoters. CRMs of the present invention can be used in combination with a wide range of suitable minimal promoters or muscle-specific proximal promoters.

Functional variants of a CRM include sequences which vary from the reference CRM element, but which substantially retain activity as muscle-specific CRMs. It will be appreciated by the skilled person that it is possible to vary the sequence of a CRM while retaining its ability to recruit suitable muscle-specific transcription factors (TFs) and thereby enhance expression. A functional variant of a CRM can comprise substitutions, deletions and/or insertions compared to a reference CRM, provided they do not render the CRM substantially non-functional.

In some embodiments, a functional variant of a CRM can be viewed as a CRM which, when substituted in place of a reference CRM in a promoter, substantially retains its activity. For example, a muscle-specific promoter which comprises a functional variant of a given CRM preferably retains at least 80% of its activity, more preferably at least 90% of its activity, more preferably at least 95% of its activity, and yet more preferably 100% of its activity (compared to the reference promoter comprising the unmodified CRM).

Suitably, functional variants of a CRM retain a significant level of sequence identity to a reference CRM. Suitably functional variants comprise a sequence that is at least 70% identical to the reference CRM, more preferably at least 80%, 90%, 95% or 99% identical to the reference CRM.

Retention of activity can be assessed by comparing expression of a suitable reporter under the control of the reference promoter with an otherwise identical promoter comprising the substituted CRM under equivalent conditions. Suitable assays for assessing muscle-specific promoter activity are disclosed herein, e.g. in the examples.

Functional variants of a given CRM can, in some embodiments, comprise functional variants of one or more of the CREs present in the reference CRM. For example, functional variants of a given CRM can comprise functional variants of 1, 2, 3, 4, 5, or 6 of the CREs present in the reference CRM.

Functional variants of a given CRM can, in some embodiments, comprise the same combination CREs as a reference CRM, but the CREs can be present in a different order from the reference CRM. It is usually preferred that the CREs are present in the same order as the reference CRM (thus, the functional variant of a CRM suitably comprises the same permutation of the CREs as set out in a reference CRM).

Functional variants of a given CRM can, in some embodiments, comprise one or more additional CREs to those present in a reference CRM. Additional CREs can be provided upstream of the CREs present in the reference CRM, downstream of the CREs present in the reference CRM, and/or between the CREs present in the reference CRM. The additional CREs can be CREs disclosed herein, or they can be other CREs. Generally, it is preferred that a functional variant of a given CRM comprises the same CREs (or functional variants thereof) and does not comprise additional CREs.

Functional variants of a given CRM can comprise one or more additional regulatory elements compared to a reference CRM. For example, they may comprise an inducible or repressible element, a boundary control element, an insulator, a locus control region, a response element, a binding site, a segment of a terminal repeat, a responsive site, a stabilizing element, a de-stabilizing element, and a splicing element, etc., provided that they do not render the CRM substantially non-functional.

Functional variants of a given CRM can comprise additional spacers between adjacent CREs or, if one or more spacers are present in the reference CRM, said one or more spacers can be longer or shorter than in the reference CRM.

It will be apparent that the CRMs as disclosed herein, or functional variants thereof, can be combined with any suitable promoter elements in order to provide a synthetic muscle-specific promoter according to the present invention.

In many instances, shorter promoter sequences are preferred, particularly for use in situations where a vector (e.g. a viral vector such as AAV) has limited capacity. Accordingly, in some embodiments the synthetic muscle-specific CRM has length of 250 or fewer nucleotides, for example 220, 200, 180, 150, 100, 75, 60, 50 or fewer nucleotides. In some particularly preferred embodiments, the synthetic muscle-specific CRM has length of 200 or fewer nucleotides.

### Promoter Elements and Functional Variants Thereof:

CREs and CRMs of the present invention can be used in combination with a wide range of suitable minimal promoters or muscle-specific proximal promoters, collectively called promoter elements.

Functional variants of promoter elements include sequences which vary from the reference promoter element, but which substantially retain activity as muscle-specific promoter element. It will be appreciated by the skilled person that it is possible to vary the sequence of a promoter element while retaining its ability to promote expression. A functional variant of a promoter element can comprise substitutions, deletions and/or insertions compared to a reference promoter element, provided they do not render the promoter element substantially non-functional.

In some embodiments, a functional variant of a promoter element can be viewed as a promoter element which, when substituted in place of a reference promoter element in a synthetic promoter, substantially retains its activity. For example, a muscle-specific synthetic promoter which comprises a functional variant of a given promoter element preferably retains at least 80% of its activity, more preferably at least 90% of its activity, more preferably at least 95% of its activity, and yet more preferably 100% of its activity (compared to the reference promoter comprising the unmodified promoter element).

Suitably, functional variants of a promoter element retain a significant level of sequence identity to a reference promoter element. Suitably functional variants comprise a sequence that is at least 70% identical to the reference promoter element, more preferably at least 80%, 90%, 95% or 99% identical to the reference promoter element.

Retention of activity can be assessed by comparing expression of a suitable reporter under the control of the reference promoter with an otherwise identical promoter comprising the substituted promoter element under equivalent conditions. Suitable assays for assessing muscle-specific promoter activity are disclosed herein, e.g. in the examples.

### Synthetic Muscle-Specific Promoters and Functional Variants Thereof

Various synthetic muscle-specific promoters are disclosed herein. A functional variant of a reference synthetic muscle-specific promoter is a promoter which comprises a sequence which varies from the reference synthetic muscle-specific promoter, but which substantially retains muscle-specific promoter activity. It will be appreciated by the skilled person that it is possible to vary the sequence of a synthetic muscle-specific promoter while retaining its ability to recruit suitable muscle-specific transcription factors (TFs) and to recruit RNA polymerase II to provide muscle-specific expression of an operably linked sequence (e.g. an open reading frame). A functional variant of a synthetic muscle-specific promoter can comprise substitutions, deletions and/or insertions compared to a reference promoter, provided such substitutions, deletions and/or insertions do not render the synthetic muscle-specific promoter substantially non-functional compared to the reference promoter.

Accordingly, in some embodiments, a functional variant of a synthetic muscle-specific promoter can be viewed as a variant which substantially retains the muscle-specific promoter activity of the reference promoter. For example, a functional variant of a synthetic muscle-specific promoter preferably retains at least 70% of the activity of the reference promoter, more preferably at least 80% of its activity, more preferably at least 90% of its activity, more preferably at least 95% of its activity, and yet more preferably 100% of its activity.

Functional variants of a synthetic muscle-specific promoter often retain a significant level of sequence similarity to a reference synthetic muscle-specific promoter. In some embodiments, functional variants comprise a sequence that is at least 70% identical to the reference synthetic muscle-specific promoter, more preferably at least 80%, 90%, 95% or 99% identical to the reference synthetic muscle-specific promoter.

Activity in a functional variant can be assessed by comparing expression of a suitable reporter under the control of the reference synthetic muscle-specific promoter with the expression of a suitable reporter under the control of the putative functional variant under equivalent conditions. Suitable assays for assessing muscle-specific promoter activity are disclosed herein, e.g. in the examples.

Functional variants of a given synthetic muscle-specific promoter can comprise functional variants of one or more CREs present in the reference synthetic muscle-specific promoter. For example, functional variant of a given CRM can comprise 1, 2, 3, 4, 5, or 6 of the CREs present in the reference CRM. Functional variants of CREs are discussed above.

Functional variants of a given synthetic muscle-specific promoter can comprise functional variants of the promoter element, or a different promoter element when compared to the reference synthetic muscle-specific promoter.

Functional variants of a given synthetic muscle-specific promoter can comprise the same CREs as a reference synthetic muscle-specific promoter, but the CREs can be present in a different order from the reference synthetic muscle-specific promoter.

Functional variants of a given synthetic muscle-specific promoter can comprise one or more additional CREs to those present in a reference synthetic muscle-specific promoter. Additional CREs can be provided upstream of the CREs present in the reference CRM, downstream of the CREs present in the reference synthetic muscle-specific promoter, and/or between the CREs present in the reference synthetic muscle-specific promoter. The additional CREs can be CREs disclosed herein, or they can be other CREs.

Functional variants of a given synthetic muscle-specific promoter can comprise one or more additional regulatory elements compared to a reference synthetic muscle-specific promoter. For example, they may comprise an inducible elements, an intronic element, a boundary control element, an insulator, a locus control region, a response element, a binding site, a segment of a terminal repeat, a responsive site, a stabilizing element, a de-stabilizing element, and a splicing element, etc., provided that they do not render the promoter substantially non-functional.

Functional variants of a given synthetic muscle-specific promoter can comprise additional spacers between adjacent CREs and promoter elements or, if one or more spacer are present in the reference synthetic muscle-specific promoter, said one or more spacers can be longer or shorter than in the reference synthetic muscle-specific promoter. Functional variant examples are provided below.

SP0522 is a functional variant of SP0502 and vice versa as SP0522 is a shorter version of SP0502. SP0523 is a functional variant of SP0515 and vice versa as SP0523 is a shorter version of SP0515. SP0524 is a functional variant of SP0521 and vice versa as SP0524 is a shorter version of SP0521.

It will be apparent that synthetic muscle-specific promoters of the present invention can comprise a synthetic muscle-specific promoter of the present invention and additional regulatory sequences. For example, they may comprise one or more additional CRMs, an inducible or repressible element, a boundary control element, an insulator, a locus control region, a response element, a binding site, a segment of a terminal repeat, a responsive site, a stabilizing element, a de-stabilizing element, and a splicing element, etc., provided that they do not render the promoter substantially non-functional.

Preferred synthetic muscle-specific promoters of the present invention exhibit muscle-specific promoter activity which is at least 15%, 20%, 25%, 30%, 40%, 50%, 60%, 70%, 80%, 90%, 100%, 125%, 150%, 175%, 200%, 250%, 300%, 350% or 400% of the activity exhibited by the CBA or RSV promoter in muscle cells. In many cases higher levels of promoter activity is preferred, but this is not always the case; thus, in some cases more moderate levels of expression may be preferred. In some cases, it is desirable to have available a range of promoters of different activity levels to allow the level of expression to by tailored to requirements; the present disclose provides promoters which are expected to provide such a range of activities. Activity of a given synthetic muscle-specific promoter of the present invention compared to CBA or RSV can be assessed by comparing muscle-specific expression of a reporter gene under control of the synthetic muscle-specific promoter with expression of the same reporter under control of the CBA or RSV promoter, when the two promoters are provided in otherwise equivalent expression constructs and under equivalent conditions.

In some embodiments a synthetic muscle-specific promoter of the invention is able to increase expression of a gene (e.g. a therapeutic gene or gene of interest) in the muscle of a subject or in a muscle cell by at least 20%, at least 40%, at least 60%, at least 80%, at least 100%, at least 200%, at least 300%, at least 500%, at least 1000% or more relative to a known muscle-specific promoter, suitably the SPc5-12 promoter (Gene Ther. 2008 November; 15 (22): 1489-99).

Preferred synthetic muscle-specific promoters of the present invention exhibit activity in non-muscle cells (e.g. Huh7 and HEK293 cells) which is 50% or less when compared to CMV-IE, preferably 25% or less than CMV-IE, more preferably 10% or less than CMV-IE, and in some cases 5% or less than CMV-IE, or 1% or less than CMV-IE.

In many instances, shorter promoter sequences are preferred, particularly for use in situations where a vector (e.g. a viral vector such as AAV) has limited capacity. Accordingly, in some embodiments the synthetic muscle-specific promoter has length of 300 or fewer nucleotides, for example, 290, 280, 270, 260, 250, 240, 230, 220, 210, 200, 150, 100, 75, 70, 68 or fewer nucleotides. In some embodiments, the synthetic muscle-specific promoter has length of 300 or fewer nucleotides, preferably 290 or fewer nucleotides, more preferably 280 or fewer nucleotides, yet more preferably 270 or fewer nucleotides. In some embodiments, the synthetic muscle-specific promoter has length of 260 or fewer nucleotides, preferably 250 or fewer nucleotides, more preferably 240 or fewer nucleotides, yet more preferably 230 or fewer nucleotides.

Particularly preferred synthetic muscle-specific promoters are those that are both short and which exhibit high levels of activity.

### Synthetic Muscle-Specific Expression Cassettes

The present invention also provides a synthetic muscle-specific expression cassette comprising a synthetic muscle-specific promoter of the present invention operably linked to a sequence encoding an expression product, suitably a gene (e.g. a transgene).

The gene typically encodes a desired gene expression product such as a polypeptide (protein) or RNA. The gene may be a full-length cDNA or genomic DNA sequence, or any fragment, subunit or mutant thereof that has at least some desired biological activity.

Where the gene encodes a protein, it can be essentially any type of protein. By way of non-limiting example, the protein can be an enzyme, an antibody or antibody fragment (e.g. a monoclonal antibody), a viral protein (e.g. REP-CAP, REV, VSV-G, or RD114), a therapeutic protein, or a toxic protein (e.g. Caspase 3, 8 or 9).

In some preferred embodiments of the present invention, the gene encodes a therapeutic expression product, preferably a therapeutic polypeptide suitable for use in treating a disease or condition associated with aberrant gene expression, optionally in the muscle, optionally in skeletal and/or cardiac muscle.

In some embodiments, therapeutic expression products include those useful in the treatment of muscle diseases. The term “muscular disease” or “muscle disease” or is, in principle, understood by the skilled person. The term relates to a disease amenable to treatment and/or prevention by administration of an active compound to a muscle, in particular to a muscle cell. In some embodiments, the muscular disease is a skeletal muscle disease. In some embodiments, the muscular disease is a cardiac muscle disease.

In some embodiments, therapeutic expression products are those useful in the treatment of Duchenne muscular dystrophy. In some embodiments, the therapeutic expression products is the DMD gene or a functional variant thereof.

In some embodiments, therapeutic expression products are those useful in the treatment of heart failure or congestive heart failure.

In some embodiments, the muscular disease is a vascular disease, a muscular dystrophy, a cardiomyopathy, a myotonia, a muscular atrophy, a myoclonus dystonia (affected gene: SGCE), a mitochondrial myopathy, a rhabdomyolysis, a fibromyalgia, and/or a myofascial pain syndrome.

In one embodiment, the disease may be cardiovascular condition or heart disease or disorder. In one embodiment, the disease may be heart failure such as congestive heart failure. In one embodiment, the disease may be selected from ischemia, arrhythmia, myocardial infarction (MI), abnormal heart contractility, non-ischemic cardiomyopathy, peripheral arterial occlusive disease, and abnormal Ca2+ metabolism, and combinations thereof. In some embodiments, the disease may be selected from the group of: congestive heart failure, cardiomyopathy, myocardial infarction, tissue ischemia, cardiac ischemia, vascular disease, acquired heart disease, congenital heart disease, atherosclerosis, dysfunctional conduction systems, dysfunctional coronary arteries, pulmonary heart hypertension. In some embodiments, the disease may be selected from congestive heart failure, coronary artery disease, myocardial infarction, myocardial ischemia, atherosclerosis, cardiomyopathy, idiopathic cardiomyopathy, cardiac arrhythmias, Danon disease, muscular dystrophy, muscle mass abnormality, muscle degeneration, infective myocarditis, drug- or toxin-induced muscle abnormalities, hypersensitivity myocarditis, an autoimmune endocarditis and congenital heart disease.

In some embodiments, the cardiomyopathy is hypertrophic cardiomyopathy, arrhythmogenic right ventricular dysplasia, dilated cardiomyopathy, restrictive cardiomyopathy, left ventricular noncompaction, Takotsubo cardiomyopathy, myocarditis, eosinophilic myocarditis, and ischemic cardiomyopathy. Preferably, the hypertrophic cardiomyopathy is CMH1 (Gene: MYH7), CMH2 (Gene: TNNT2), CMH3 (Gene: TPM1), CMH4 (Gene: MYBPC3), CMH5, CMH6 (Gene: PRKAG2), CMH7 (Gene: TNNI3), CMH8 (Gene: MYL3), CMH9 (Gene: TTN), CMH10 (Gene: MYL2), CMH11 (Gene: ACTC1), or CMH12 (Gene: CSRP3). Preferably, the arrhythmogenic right ventricular dysplasia is ARVD1 (Gene: TGFB3), ARVD2 (Gene: RYR2), ARVD3, ARVD4, ARVD5 (Gene: TMEM43), ARVD6, ARVD7 (Gene: DES), ARVD8 (Gene: DSP), ARVD9 (Gene: PKP2), ARVD10 (Gene: DSG2), ARVD11 (Gene: DSC2), and/or ARVD12 (Gene: JUP).

In some embodiments, the muscular disease is a vascular disease. Vascular disease may be coronary artery disease, peripheral arterial disease, cerebrovascular disease, renal artery stenosis or aortic aneurysm. In some embodiments, the muscular disease may be cardiomyopathy. The cardiomyopathy may be hypertensive heart disease, heart failure (such as congestive heart failure), pulmonary heart disease, cardiac dysrhythmias, inflammatory heart disease (such as endocarditis, inflammatory cardiomegaly, myocarditis), valvular heart disease, congenital heart disease and rheumatic heart disease.

In some embodiments, the muscular dystrophy is Duchenne muscular dystrophy (gene affected: DMD), Becker muscular dystrophy (gene affected: DMD), Limb girdle muscular dystrophy (Subtypes and affected genes: LGMD1A (Gene: TTID), LGMD1B (Gene: LMNA), LGMD1C (Gene: CAV3), LGMD1D (Gene: DNAJB6), LGMD1E (Gene: DES), LGMD1F (Gene: TNP03), LGMD1G (Gene: HNRPDL), LGMD1H, LGMD2A (Gene: CAPN3), LGMD2B (Gene: DYSF), LGMD2C (Gene: SGCG), LGMD2D (Gene: SGCA), LGMD2E (Gene: SGCB), LGMD2F (Gene: SGCD), LGMD2G (Gene: TCAP), LGMD2H (Gene: TRIM32), LGMD2I (Gene: FKRP), LGMD2J (Gene: TTN), LGMD2K (Gene: POMT1), LGMD2L (Gene: AN05), LGMD2M (Gene: FKTN), LGMD2N (Gene: POMT2), LGMD20 (Gene: POMGNT1), LGMD2Q (Gene: PLEC1)), Congenital muscular dystrophy, Duchenne muscular dystrophy, Emery-Dreifuss muscular dystrophy, Distal muscular dystrophy (Subtypes and affected genes: Miyoshi myopathy (Gene: DYSF), Distal myopathy with anterior tibial onset (Gene: DYSF), Welander distal myopathy (Gene: TIA1), Gowers-Laing distal myopathy (Gene: MYH7), Nonaka distal myopathy, hereditary inclusion-body myositis type 1, distal myopathy with vocal cord and pharyngeal weakness, ZASP-related myopathy), Facioscapulohumeral muscular dystrophy (Subtypes and affected genes: Type 1 (Gene: DUX4), Type 2 (Gene: SMCHD1)), Oculopharyngeal muscular dystrophy (affected gene: PABPN1), and/or myotonic dystrophy (Subtypes and affected genes: DM1 (Gene: DMPK) and DM2 (Gene: ZNF9)).

In some embodiments, the myotonia is congenital myotonia (affected gene: CLCN1; subtypes: Type Thomsen, Type Becker), Potassium-aggravated myotonia and/or Paramyotonia congenital (affected gene: SCN4A).

In some embodiments, the muscular disease is Duchenne muscular dystrophy (Gene: DMD), a myotubular myopathy (Gene: MTM1), Spinal muscular atrophy (Gene: SMA), Glycogen storage disease type II (Pompe disease, Gene: GAA), or a cardiomyopathy.

In some embodiments, the disease may be Hyperkalemia periodic paralysis or Hypokalemic periodic paralysis.

In some embodiments, the disease may be congenital myopathy selected from nemaline myopathy, Multi/minicore myopathy and Centronuclear myopathy.

In some embodiments, the disease may be selected from inflammatory myopathy, metabolic myopathy, Brody myopathy or Hereditary inclusion body myopathy.

In some embodiments the gene encodes a non-disease mediating variant, e.g. a wildtype variant of at least one human gene selected from the group consisting of DMD, GALGT2, SMA, GAA, MTM1, TTID, LMNA, CAV3, DNAJB6, DES, TNP03, HNRPDL, CAPN3, DYSF, SGCG, SGCA, SGCB, SGCD, TCAP, TRIM32, FKRP, TTN, POMT1, AN05, FKTN, POMT2, PFEC1, TIA1, MYH7, DUX4, SMCHD, PABPN1, DMPK, MBNL1, ZNF9, CFCN1, SCN4A, MYH7, TNNT2, TPM1, MYBPC3, PRKAG2, TNNI3, MYF3, TTN, MYF2, ACTC1, CSRP3, TGFB3, RYR2, TMEM43, DES, DSP, PKP2, DSG2, DSC2, JUP, CNBP, CLCN1, SEPN1, RYR1 and HYPP.

In some embodiments the gene encodes a synthetic wildtype variant of at least one human gene selected from the group consisting of consisting of DMD GALGT2, SMA, GAA, MTM1, TTID, LMNA, CAV3, DNAJB6, DES, TNP03, HNRPDL, CAPN3, DYSF, SGCG, SGCA, SGCB, SGCD, TCAP, TRIM32, FKRP, TTN, POMT1, AN05, FKTN, POMT2, PFEC1, TIA1, MYH7, DUX4, SMCHD, PABPN1, DMPK, MBNL1, ZNF9, CFCN1, SCN4A, MYH7, TNNT2, TPM1, MYBPC3, PRKAG2, TNNI3, MYF3, TTN, MYF2, ACTC1, CSRP3, TGFB3, RYR2, TMEM43, DES, DSP, PKP2, DSG2, DSC2, JUP, CNBP, CLCN1, SEPN1, RYR1 and HYPP.

Further exemplary muscle tissue-related diseases include but are not limited to Acid Maltase Deficiency (AMD), alpha-1 antitrypsin deficiency, Amyotrophic Lateral Sclerosis (ALS), Andersen-Tawil Syndrome, Becker Muscular Dystrophy (BMD), Becker Myotonia Congenita, Bethlem Myopathy, Cardiovascular Disease, Carnitine Deficiency, Carnitine Palmityl Transferase Deficiency (CPT Deficiency), Central Core Disease (CCD), Centronuclear Myopathy, Charcot-Marie-Tooth Disease (CMT), Congenital Myasthenic Syndromes (CMS), Congenital Myotonic Dystrophy, Congestive Heart Failure, Cori Disease (Debrancher Enzyme Deficiency), Debrancher Enzyme Deficiency, Dejerine-Sottas Disease (DSD), Dermatomyositis (DM), Endocrine Myopathies, Eulenberg Disease (Paramyotonia Congenita), Forbes Disease (Debrancher Enzyme Deficiency), Friedreich's Ataxia (FA), Glycogenosis Type 10, Glycogenosis Type 11, Glycogenosis Type 2, Glycogenosis Type 3, Glycogenosis Type 5, Glycogenosis Type 7, Glycogenosis Type 9, Gowers-Laing Distal Myopathy, Hauptmann-Thanheuser MD (Emery-Dreifuss Muscular Dystrophy), Hereditary Inclusion-Body Myositis, Hereditary Motor and Sensory Neuropathy (Charcot-Marie-Tooth Disease), Hyperthyroid Myopathy, Hypothyroid Myopathy, Inclusion-Body Myositis (IBM), Inherited Myopathies, Integrin-Deficient Congenital Muscular Dystrophy, Lactate Dehydrogenase Deficiency, Lambert-Eaton Myasthenic Syndrome (LEMS), McArdle Disease (Phosphorylase Deficiency), Metabolic Diseases of Muscle, Mitochondrial Myopathy, Miyoshi Distal Myopathy, Motor Neurone Disease, Muscle-Eye-Brain Disease, Myasthenia Gravis (MG), Myoadenylate Deaminase Deficiency, Myofibrillar Myopathy, Myophosphorylase Deficiency, Myotonia Congenita (MC), Myotonic Muscular Dystrophy (MMD), Myotubular Myopathy (MTM or MM), Nemaline Myopathy, Nonaka Distal Myopathy, Oculopharyngeal Muscular Dystrophy (OPMD), Paramyotonia Congenita, Pearson Syndrome, Periodic Paralysis, Peroneal Muscular Atrophy (Charcot-Marie-Tooth Disease), Phosphofructokinase Deficiency, Phosphogly cerate Kinase Deficiency, Phosphogly cerate Mutase Deficiency, Phosphorylase Deficiency, Phosphorylase Deficiency, Polymyositis (PM), Pompe Disease (Acid Maltase Deficiency), Progressive External Ophthalmoplegia (PEO), Rod Body Disease (Nemaline Myopathy), Spinal Muscular Atrophy (SMA), Spinal-Bulbar Muscular Atrophy (SBMA), Steinert Disease (Myotonic Muscular Dystrophy), Tarui Disease (Phosphofructokinase Deficiency), Thomsen Disease (Myotonia Congenita), Ullrich Congenital Muscular Dystrophy, Walker-Warburg Syndrome (Congenital Muscular Dystrophy), Welander Distal Myopathy, and ZASP-Related Myopathy.

In some embodiments, the muscular disease is a cardiac muscle disease. In some embodiments, the muscular disease is congestive heart failure.

In some embodiments, useful expression products include dystrophins (including micro-dystrophins), beta 1,4-n-acetylgalactosamine galactosyltransferase (GALGT2), carbamoyl synthetase I, alpha-1 antitrypsin, ornithine transcarbamylase, arginosuccinate synthetase, arginosuccinate lyase, arginase, fumarylacetacetate hydrolase, phenylalanine hydroxylase, glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, and a cystic fibrosis transmembrane regulator (CFTR).

Still other useful expression products include enzymes useful in enzyme replacement therapy, and which are useful in a variety of conditions resulting from deficient activity of enzyme. For example, enzymes containing mannose-6-phosphate may be utilized in therapies for lysosomal storage diseases (e.g., a suitable gene includes that encoding β-glucuronidase (GUSB)).

In some embodiments, exemplary polypeptide expression products include neuroprotective polypeptides and anti-angiogenic polypeptides. Suitable polypeptides include, but are not limited to, glial derived neurotrophic factor (GDNF), fibroblast growth factor 2 (FGF-2), nurturin, ciliary neurotrophic factor (CNTF), nerve growth factor (NGF; e.g., nerve growth factor-. Beta.), brain derived neurotrophic factor (BDNF), neurotrophin-3 (NT-3), neurotrophin-4 (NT-4), neurotrophin-6 (NT-6), epidermal growth factor (EGF), pigment epithelium derived factor (PEDF), a Wnt polypeptide, soluble Fit-1, angiostatin, endostatin, VEGF, an anti-VEGF antibody, a soluble VEGFR, Factor VIII (FVIII), Factor IX (FIX), and a member of the hedgehog family (sonic hedgehog, Indian hedgehog, and desert hedgehog, etc.).

In some embodiments, useful therapeutic expression product include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor alpha superfamily, including TGFa., activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

In some embodiments, useful expression products include proteins that regulate the immune system including, without limitation, cytokines and lymphokines such as thrombopoietin (TPO), interleukins (IL) IL-1 through IL-25 (including IL-2, IL-4, IL-12 and IL-18), monocyte chemoattractant protein, leukemia inhibitory factor, granulocyte-macrophage colony stimulating factor, Fas ligand, tumor necrosis factors alpha and beta., interferons (alpha, beta, and gamma), stem cell factor, flk-2/flt3 ligand. Gene products produced by the immune system are also useful in the present invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

In some embodiments, useful expression product include any one of the receptors for the hormones, growth factors, cytokines, lymphokines, regulatory proteins and immune system proteins. Useful heterologous nucleic acid sequences also include receptors for cholesterol regulation and/or lipid modulation, including the low-density lipoprotein (LDL) receptor, high density lipoprotein (HDL) receptor, the very low density lipoprotein (VLDL) receptor, and scavenger receptors. The invention also encompasses the use of gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP-2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

In some embodiments, useful expression products include those used for treatment of hemophilia, including hemophilia B (including Factor IX) and hemophilia A (including Factor VIII and its variants, such as the light chain and heavy chain of the heterodimer and the B-deleted domain; U.S. Pat. Nos. 6,200,560 and 6,221,349).

In some embodiments, the useful expression product may be a modulator of phosphatase activity, e.g., type 1 phosphatase activity. The modulator may be a protein that inhibits phosphatase activity, e.g., type 1 phosphatase activity. The modulator may be a nucleic acid that increases expression of an endogenous nucleic acid that encodes a protein that inhibits phosphatase activity such as a transcription factor. The modulator may be a regulatory sequence that integrates in or near the endogenous nucleic acid that encodes a protein that inhibits phosphatase activity. The modulator may be a nucleic acid that can provide a nucleic acid modulator of gene expression such as a siRNA.

In some embodiments, the useful expression product may be inhibitor of protein phosphate 1 (PP1) e.g., a I-1 polypeptide. The phosphatase inhibitor-1 (or “I-1”) protein is an endogenous inhibitor of type 1 phosphatase. Increasing I-1 levels or activity can restore β-adrenergic responsiveness in failing human cardiomyocytes. Suitably, the I-1 protein may be constitutively active such as a I-1 protein where threonine 35 is replaced with glutamic acid instead of aspartic acid. The therapeutic expression product may be any one or more of the inhibitors selected from: phosphatase inhibitor 2 (PP2); okadaic acid or caliculin; and nippl which is an endogenous nuclear inhibitor of protein phosphatase 1.

In some embodiments, the useful expression product may be any protein that modulates cardiac activity such as a phosphatase type 1 inhibitor, e.g., 1-1 or a sacroplasmic reticulum Ca2+ ATPase (SERCA), e.g., SERCA1 (e.g., 1a or 1b), SERCA2 (e.g., 2a or 2b), or SERCA3.

In some embodiments, the useful expression product may be a nucleic acid sequence encoding a mutant form of phosphatase inhibitor-1 protein, wherein the mutant form comprises at least one amino acid at a position that is a PKC-α phosphorylation site in the wild type, wherein the at least one amino acid is constitutively unphosphorylated or mimics an unphosphorylated state in the mutant form. The therapeutic expression product may be adenylyl cyclase 6 (AC6, also referred to as adnenylyl cyclase VI), S100A1, β-adrenergic receptor kinase-ct (βARKct), sarco/endoplasmic reticulum (SR) Ca-ATPase (SERCA2a), IL-18, VEGF, VEGF activators, urocortins, and B-cell lymphoma 2 (Bcl2)-associated anthanogene-3 (BAG3).

In some embodiments, the useful expression product may be an inhibitor of a cytokine such as an IL-18 inhibitor. The therapeutic expression product may be a beta-adrenergic signalling protein (beta-ASPs) (including beta-adrenergic receptors (beta-Ars), G-protein receptor kinase inhibitors (GRK inhibitors) and adenylylcyclases (Acs)) to enhance cardiac function.

In some embodiments, the useful expression product may be an angiogenic protein. Angiogenic proteins promote development and differentiation of blood vessels. Examples of angiogenic proteins include members of the fibroblast growth factor (FGF) family such as aFGF (FGF-1), bFGF (FGF-2), FGF-4 (also known as “hst/KS3”), FGF-5 and FGF-6, the vascular endothelial growth factor (VEGF) family, the platelet-derived growth factor (PDGF) family, the insulin-like growth factor (IGF) family, and others.

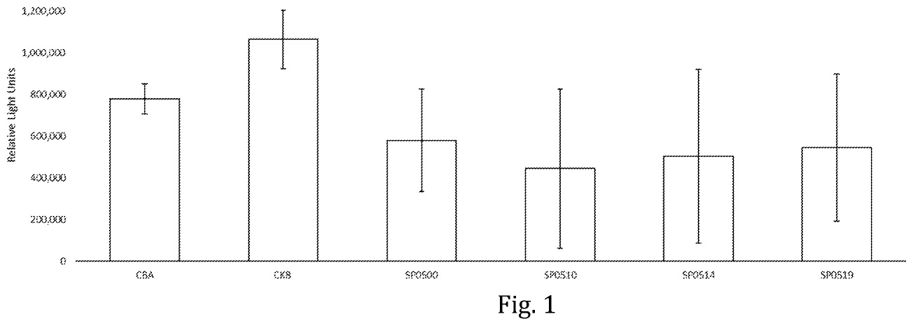

In some embodiments, useful expression products include non-naturally occurring polypeptides, such as chimeric or hybrid polypeptides having a non-naturally occurring amino acid sequence containing insertions, deletions or amino acid substitutions. In some embodiments, the expression product may be a synthetic dysferlin protein such as Nano-Dysferlin as detailed in table 1 and FIG. 1A of (Llanga et al., 2017) which is a shorter version of wild type dysferlin.

Further suitable expression products include micro RNA (miRNA), interfering RNA, antisense RNA, ribozymes, and aptamers.

In some preferred embodiments, the expression product is an inhibitor of protein phosphate 1 (PP1).

In some embodiments of the invention, the synthetic muscle-specific expression cassette comprises a gene useful for gene editing, e.g. a gene encoding a site-specific nuclease, such as a meganuclease, zinc finger nuclease (ZFN), transcription activator-like effector-based nuclease (TALEN), or the clustered regularly interspaced short palindromic repeats system (CRISPR-Cas). Suitably the site-specific nuclease is adapted to edit a desired target genomic locus by making a cut (typically a site-specific double-strand break) which is then repaired via non-homologous end-joining (NHEJ) or homology dependent repair (HDR), resulting in a desired edit. The edit can be the partial or complete repair of a gene that is dysfunctional, or the knock-down or knock-out of a functional gene. Alternatively, the edit can be via base editing or prime editing, using suitable systems which are known in the art.

In some embodiments of the invention, the synthetic muscle-specific expression cassette comprises a gene useful for gene modulation e.g. DNA binding protein fused to a gene repressor or a gene activator. For example, a zinc finger protein fused to a gene repressor or an gene activator or an endonuclease deficient cas9 fused to a gene repressor or an gene activator.

Suitably the synthetic muscle-specific expression cassette comprises sequences providing or coding for one or more of, and preferably all of, a ribosomal binding site, a start codon, a stop codon, and a transcription termination sequence. Suitably the expression cassette comprises a nucleic acid encoding a posttranscriptional regulatory element. Suitably the expression cassette comprises a nucleic acid encoding a polyA element.

### Vectors and Viral Particles

The present invention further provides a vector comprising a synthetic muscle-specific promoter, or expression cassette according to the present invention.

In some embodiments of the invention, the vector is a plasmid. Such a plasmid may include a variety of other functional nucleic acid sequences, such as one or more selectable markers, one or more origins of replication, multiple cloning sites and the like. In some embodiments of the invention, the vector is a viral vector.

In some embodiments of the invention, the vector is an expression vector for expression in eukaryotic cells. Examples of eukaryotic expression vectors include, but are not limited to, pW-LNEO, pSV2CAT, pOG44, pXTI and pSG available from Stratagene; pSVK3, pBPV, pMSG and pSVL available from Amersham Pharmacia Biotech; and pCMVDsRed2-express, PIRES2-DsRed2, pDsRed2-Mito, pCMV-EGFP available from Clontech. Many other vectors are well-known and commercially available. For mammalian cells adenoviral vectors, the pSV and the pCMV series of vectors are particularly well-known non-limiting examples. There are many well-known yeast expression vectors including, without limitation, yeast integrative plasmids (Yip) and yeast replicative plasmids (Yrp). For plants the Ti plasmid of agrobacterium is an exemplary expression vector, and plant viruses also provide suitable expression vectors, e.g. tobacco mosaic virus (TMV), potato virus X, and cowpea mosaic virus.

In some preferred embodiments, the vector is a gene therapy vector. Various gene therapy vectors are known in the art, and mention can be made of AAV vectors, adenoviral vectors, retroviral vectors and lentiviral vectors. Where the vector is a gene therapy vector the vector preferably comprises a nucleic acid sequence operably linked to the synthetic muscle-specific promoter of the invention that encodes a therapeutic product, suitably a therapeutic protein. The therapeutic protein may be a secretable protein. Non-limiting examples of secretable proteins are discussed above, and exemplary secretable therapeutic proteins, include clotting factors, such as factor VIII or factor IX, insulin, erythropoietin, lipoprotein lipase, antibodies or nanobodies, growth factors, cytokines, chemokines, plasma factors, toxic proteins, etc.

In some embodiments of the invention, the vector is a viral vector, such as a retroviral, lentiviral, adenoviral, or adeno-associated viral (AAV) vector. In some preferred embodiments the vector is an AAV vector. In some preferred embodiments the AAV has a serotype suitable for muscle transduction. In some embodiments, the AAV is selected from the group consisting of: AAV2, AAV5, AAV6, AAV7, AAV8, AAV9 BNP116, rh10, AAV2.5, AAV2i8, AAVDJ8 and AAV2G9, or derivatives thereof. AAV vectors are preferably used as self-complementary, double-stranded AAV vectors (scAAV) in order to overcome one of the limiting steps in AAV transduction (i.e. single-stranded to double-stranded AAV conversion), although the use of single-stranded AAV vectors (ssAAV) is also encompassed herein. In some embodiments of the invention, the AAV vector is chimeric, meaning it comprises components from at least two AAV serotypes, such as the ITRs of an AAV2 and the capsid protein of an AAV5. AAV9 is known to effectively transduce skeletal muscle and cardiac muscle particularly effectively, and thus AAV9 and derivatives thereof are of particular interest for targeting skeletal and cardiac muscle. AAV1, AAV6, AAV7 and AAV8 are also known to target skeletal muscle, and thus these AAV serotypes and derivates thereof are also of particular interest for targeting skeletal muscle. AAV1 and AAV8 are also known to target cardiac muscle, and thus these AAV serotypes and derivates thereof are also of particular interest for targeting cardiac muscle. In some embodiments, the rAAV vector is an AAV3b serotype, including, but not limited to, an AAV3b265D virion, an AAV3b265D549A virion, an AAV3b549A virion, an AAV3bQ263Y virion, or an AAV3bSASTG virion (i.e., a virion comprising an AAV3b capsid comprising Q263A/T265 mutations). In some embodiments, the virion can be rational haploid, or a chimeric or any mutant, such as capsids can be tailored for increased update at a desired location, e.g., the heart. Other capsids can include capsids from any of the known AAV serotypes, including AAV1, AAV3, AAV4, AAV5, AAV7, AAV10, etc.

The invention further provides recombinant virions (viral particles) comprising a vector as described above.

### Pharmaceutical Compositions

The vectors or virions of the present invention may be formulated in a pharmaceutical composition with a pharmaceutically acceptable excipient, i.e., one or more pharmaceutically acceptable carrier substances and/or additives, e.g., buffers, carriers, excipients, stabilisers, etc. The pharmaceutical composition may be provided in the form of a kit. Pharmaceutical compositions and delivery systems appropriate for the AAV vectors or and methods and uses of are known in the art.

Accordingly, a further aspect of the invention provides a pharmaceutical composition comprising a vector or virion as described herein.

### Therapeutic and Other Methods and Uses

The present invention also provides a synthetic muscle-specific promoter, expression cassette, vector, virion or pharmaceutical composition according to various aspects of the present invention for use in the treatment of a disease, preferably a disease associated with aberrant gene expression, optionally in the muscle (e.g. a genetic muscle disease). In one embodiment, the present invention provides a synthetic muscle-specific promoter, expression cassette, vector, virion or pharmaceutical composition according to various aspects of the present invention for use in the treatment of a skeletal muscle disease. In one embodiment, the present invention also provides a synthetic muscle-specific promoter, expression cassette, vector, virion or pharmaceutical composition according to various aspects of the present invention for use in the treatment of a cardiac muscle disease.

Relevant conditions, diseases and therapeutic expression products are discussed above.

The present invention also provides a synthetic muscle-specific promoter, expression cassette, vector, virion according to the various aspects of the present invention for use in the manufacture of a pharmaceutical composition for treatment of any condition or disease mentioned herein.

The present invention further provides a cell comprising a synthetic muscle-specific promoter, expression cassette, vector, virion according to the various aspects of the invention. Suitably the cell is a eukaryotic cell. The eukaryotic cell can suitably be a fungal cell (e.g. yeast cell), an animal (metazoan) cell (e.g. a mammalian cell), or a plant cell. Alternatively, the cell may be a prokaryotic cell.

In some embodiments of the invention, the cell is ex vivo, e.g. in cell culture. In other embodiments of the invention the cell may be part of a tissue or multicellular organism.

In a preferred embodiment, the cell is a muscle cell (myocyte), which may be ex vivo or in vivo. In a preferred embodiment, the cell is a cardiac muscle cell, which may be ex vivo or in vivo. In an alternative preferred embodiment, the cell is a skeletal muscle cell, which may be ex vivo or in vivo. The muscle cell may be a primary muscle cell or a cell of a muscle-derived cell line, e.g. an immortalised cell line. The cell may be present within a muscle tissue environment (e.g. within a muscle of an animal) or may be isolated from muscle tissue, e.g. it may be in cell culture. Suitably the cell is a human cell.

The skeletal muscle cells may be from fast twitch or slow twitch muscles.

The cardiac muscle cells may be selected from ventricular cardiomyocytes, atrial cardiomyocytes, cardiac fibroblasts, or endothelial cells (EC) in the heart, as well as peri-vascular cells and pacemaker cells.

The synthetic muscle-specific promoter, expression cassette, or vector, according to the invention may be inserted into the genome of the cell, or it may be episomal (e.g. present in an episomal vector).

In a further aspect, the present invention provides a method for producing an expression product, the method comprising providing a synthetic muscle-specific expression cassette according to the present invention (preferably in a vector as set out above) in a cell, preferably a muscle cell, and expressing the gene present in the synthetic muscle-specific expression cassette. The method suitably comprises maintaining said muscle cell under suitable conditions for expression of the gene. In culture this may comprise incubating the cell, or tissue comprising the cell, under suitable culture conditions. The expression may of course be in vivo, e.g. in one or more cells in the muscle of a subject. In one embodiment, the muscle cell/s are cardiac muscle cell/s. In one embodiment, the muscle cell/s are skeletal muscle cell/s.

Suitably the method comprises the step of introducing the synthetic muscle-specific expression cassette into the muscle cell. A wide range of methods of transfecting muscle cells are well-known in the art. A preferred method of transfecting muscle cells is transducing the cells with a viral vector comprising the synthetic muscle-specific expression cassette, e.g. an AAV vector.

It will be evident to the skilled person that a synthetic muscle-specific promoter, expression cassette, vector or virion according to various aspects of the invention may be used for gene therapy. Accordingly, the use of the such nucleic acid constructs in gene therapy forms part of the present invention.

The invention thus provides, in some embodiments, an expression cassette, vector or virion according to the present invention for use in gene therapy in a subject, preferably gene therapy through muscle-specific expression of a therapeutic gene. Suitably through skeletal muscle-specific expression of a therapeutic gene and/or cardiac muscle-specific expression of a therapeutic gene. The therapy may involve treatment of a disease through secretion of a therapeutic product from muscle cells, suitably a disease involving aberrant gene expression in the muscle such as the diseases discussed above.

The present invention also provides a method of expressing a therapeutic transgene in a muscle cell, the method comprising introducing into the muscle cell an expression cassette or vector according to the present invention. The muscle cell can be in vivo or ex vivo. In one embodiment, the muscle cell/s are cardiac muscle cell/s. In one embodiment, the muscle cell/s are skeletal muscle cell/s.

The present invention also provides a method of gene therapy of a subject, preferably a human, in need thereof, the method comprising:
administering to the subject (suitably introducing into the muscle of the subject) a synthetic muscle-specific expression cassette, vector, virion or pharmaceutical composition of the present invention, which comprises a gene encoding a therapeutic product.

In one embodiment, the muscle is cardiac muscle. In one embodiment, the muscle is skeletal muscle. In one embodiment, the muscle is a cardiac and/or skeletal muscle. The method suitably comprises expressing a therapeutic amount of the therapeutic product from the gene in the muscle of said subject. Various conditions and diseases that can be treated are discussed above. In one embodiment, the muscle is cardiac muscle. In one embodiment, the muscle is skeletal muscle.

Genes encoding suitable therapeutic products are discussed above.

The method suitably comprises administering a vector or virion according to the present invention to the subject. Suitably the vector is a viral gene therapy vector, for example an AAV vector.

In some embodiments, the method comprises administering to the subject a synthetic muscle-specific viral gene therapy vector (i.e., a viral gene therapy vector comprising a muscle-specific promoter described herein), which comprises a gene encoding a therapeutic product.

In some embodiments, the method comprises administering the viral gene therapy vector systemically. Systemic administration may be enteral (e.g. oral, sublingual, and rectal) or parenteral (e.g. injection). Preferred routes of injection include intravenous, intramuscular, subcutaneous, intra-arterial, intra-articular, intrathecal, and intradermal injections. In some preferred embodiments, the viral gene therapy vector is administered to the subject by anterograde epicardial coronary artery infusion (AECAI). In some particularly preferred embodiments, the viral gene therapy vector is administered to the subject by anterograde epicardial coronary artery infusion (AECAI) through a percutaneous femoral access.

In some embodiments, the subject has heart failure. In some embodiments, the method of gene therapy of the subject is for treatment of heart failure.

In some embodiments, the method comprises administering to the subject a synthetic muscle-specific viral gene therapy vector for treatment of heart failure via anterograde epicardial coronary artery infusion (AECAI), wherein the vector comprises a gene encoding a therapeutic product.

In some embodiments, the viral gene therapy vector may be administered concurrently or sequentially with one or more additional therapeutic agents or with one or more saturating agents designed to prevent clearance of the vectors by the reticular endothelial system.

Where the vector is an AAV vector, the dosage of the vector may be from 1×10¹⁰ gc/kg to 1×10¹⁵ gc/kg or more, suitably from 1×10¹² gc/kg to 1×10¹⁴ gc/kg, suitably from 5×10¹² gc/kg to 5×10¹³ gc/kg.

In general, the subject in need thereof will be a mammal, and preferably primate, more preferably a human. Typically, the subject in need thereof will display symptoms characteristic of a disease. The method typically comprises ameliorating the symptoms displayed by the subject in need thereof, by expressing the therapeutic amount of the therapeutic product.

Gene therapy protocols for therapeutic gene expression in target cells in vitro and in vivo, are well-known in the art and will not be discussed in detail here. Briefly, they include intramuscular injection, interstitial injection, instillation in airways, application to endothelium, intra-hepatic parenchyme, and intravenous or intra-arterial administration (e.g. intra-hepatic artery, intra-hepatic vein) of plasmid DNA vectors (naked or in liposomes) or viral vectors. Various devices have been developed for enhancing the availability of DNA to the target cell. While a simple approach is to contact the target cell physically with catheters or implantable materials containing the relevant vector, more complex approaches can use jet injection devices and suchlike. Gene transfer into mammalian muscle cells has been performed using both ex vivo and in vivo procedures. The ex vivo approach typically requires harvesting of the muscle cells, in vitro transduction with suitable expression vectors, followed by reintroduction of the transduced myocytes the muscle. In vivo gene transfer has been achieved by injecting DNA or viral vectors into the muscle. Anterograde epicardial coronary artery infusion may be used for injecting DNA or viral vectors in close proximity to the heart.

According to some preferred embodiments, the methods set out above may be used for the treatment of a subject with a muscle-related disease as discussed above, e.g. a muscular dystrophy or congestive heart failure.

### Definitions and General Points

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Ausubel, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); U.S. Pat. No. 4,683,195; Nucleic Acid Hybridization (Harries and Higgins eds. 1984); Transcription and Translation (Hames and Higgins eds. 1984); Culture of Animal Cells (Freshney, Alan R. Liss, Inc., 1987); Immobilized Cells and Enzymes (IRL Press, 1986); Perbal, A Practical Guide to Molecular Cloning (1984); the series, Methods in Enzymology (Abelson and Simon, eds.-in-chief, Academic Press, Inc., New York), specifically, Vols. 154 and 155 (Wu et al. eds.) and Vol. 185, “Gene Expression Technology” (Goeddel, ed.); Gene Transfer Vectors For Mammalian Cells (Miller and Calos eds., 1987, Cold Spring Harbor Laboratory); Immunochemical Methods in Cell and Molecular Biology (Mayer and Walker, eds., Academic Press, London, 1987); Handbook of Experimental Immunology, Vols. I-IV (Weir and Blackwell, eds., 1986); and Manipulating the Mouse Embryo, (Cold Spring Harbor Laboratory Press, Cold Spring Harbor, N.Y., 1986).

To facilitate the understanding of this invention, a number of terms are defined or explained below. Terms used herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “muscle” is well understood by the skilled person. Preferably, the muscle is a skeletal muscle (including the diaphragm) or a heart muscle. The promoters of the present invention can be active in skeletal muscle and/or cardiac muscle. Preferably, the muscle is a muscle of a vertebrate, more preferably of a mammal, even more preferably of a human subject. Preferably, the muscle is a striated muscle.

The term “muscle cell” or “myocyte” relates in the present to cells which are found in muscles (muscle tissue) or which are derived from muscle tissue. Muscle cells can be primary cells or a cell line (such as C2C12 or H2K cells (skeletal muscle cell line) or H9C2 cells (cardiac cell line)). The muscle cells can be in in vivo (e.g. in muscle tissue) or in vitro (e.g. in cell culture). Myocytes as found in muscle tissue are typically long, tubular cells that develop from myoblasts to form muscles in a process known as myogenesis. The term muscle cells or myocytes as used herein includes myocytes from skeletal muscle and from cardiac muscle (cardiomyocytes). The promoters of the present invention can be active in skeletal muscle cells and/or cardiac muscle cells.

The term “cis-regulatory element” or “CRE”, is a term well-known to the skilled person, and means a nucleic acid sequence such as an enhancer, promoter, insulator, or silencer, that can regulate or modulate the transcription of a neighbouring gene (i.e. in cis). CREs are found in the vicinity of the genes that they regulate. CREs typically regulate gene transcription by binding to TFs, i.e. they include TFBS. A single TF may bind to many CREs, and hence control the expression of many genes (pleiotropy). CREs are usually, but not always, located upstream of the transcription start site (TSS) of the gene that they regulate. “Enhancers” in the present context are CREs that enhance (i.e. upregulate) the transcription of genes that they are operably associated with, and can be found upstream, downstream, and even within the introns of the gene that they regulate. Multiple enhancers can act in a coordinated fashion to regulate transcription of one gene. “Silencers” in this context relates to CREs that bind TFs called repressors, which act to prevent or downregulate transcription of a gene. The term “silencer” can also refer to a region in the 3′ untranslated region of messenger RNA, that bind proteins which suppress translation of that mRNA molecule, but this usage is distinct from its use in describing a CRE. Generally, the CREs of the present invention are muscle-specific, or skeletal muscle-specific enhancer elements (often referred to as muscle-specific, or skeletal muscle-specific CREs, or muscle-specific, or skeletal muscle-specific CRE enhancers, or suchlike). In the present context, it is preferred that the CRE is located 2500 nucleotides or less from the transcription start site (TSS), more preferably 2000 nucleotides or less from the TSS, more preferably 1500 nucleotides or less from the TSS, and suitably 1000, 750, 500, 250, 200, 150, or 100 nucleotides or less from the TSS. CREs of the present invention are preferably comparatively short in length, preferably 250 nucleotides or less in length, for example they may be 200, 175, 150, 90, 80, 70, 60 or 50 nucleotides or less in length. The CREs of the present invention are typically provided in combination with an operably linked promoter element, which can be a minimal promoter or proximal promoter; the CREs of the present invention enhance muscle-specific, or skeletal muscle-specific activity of the promoter element. In any of the combinations of CREs, or functional variants thereof, disclosed herein, some or all of the recited CREs and promoter elements may suitably be positioned adjacent to one other in the promoter (i.e. without any intervening CREs or other regulatory elements). The CREs may be contiguous or non-contiguous (i.e. they can be positioned immediately adjacent to one another or they can be separated by a spacer or other sequence). The CRE's may be in any order. In some preferred embodiments, the CREs, or functional variants thereof, are provided in the recited order and are adjacent to one another. For example, the synthetic muscle-specific regulatory nucleic acid may comprise CRE0077 immediately upstream of CRE0075, and so forth. In some embodiments it is preferred that some or all of the CREs are contiguous.

The term “cis-regulatory module” or “CRM” means a functional regulatory nucleic acid module, which usually comprises two or more CREs; in the present invention the CREs are typically muscle-specific, or skeletal muscle-specific enhancers and thus the CRM is a synthetic muscle-specific, or skeletal muscle-specific regulatory nucleic acid. Thus, in the present application a CRM typically comprises a plurality of muscle-specific, or skeletal muscle-specific CREs. Typically, the multiple CREs within the CRM act together (e.g. additively or synergistically) to enhance the transcription of a gene that a synthetic promoter comprising the CRM is operably associated with. There is considerable scope to shuffle (i.e. reorder), invert (i.e. reverse orientation), and alter spacing of CREs within a CRM. Accordingly, functional variants of CRMs of the present invention include, inter alia, variants of the referenced CRMs wherein CREs within them have been shuffled and/or inverted, and/or the spacing between CREs has been altered.

As used herein, the phrase “promoter” refers to a region of DNA that generally is located upstream of a nucleic acid sequence to be transcribed that is needed for transcription to occur, i.e. which initiates transcription. Promoters permit the proper activation or repression of transcription of a coding sequence under their control. A promoter typically contains specific sequences that are recognized and bound by plurality of TFs. TFs bind to the promoter sequences and result in the recruitment of RNA polymerase, an enzyme that synthesizes RNA from the coding region of the gene. Many diverse promoters are known in the art.

In some cases, the term “promoter” or “composite promoter” is used herein to refer to a combination of a promoter and additional regulatory elements, e.g. regulatory sequences located immediately downstream of the transcription start site (TSS), for example a 5′ UTR and or a 5′UTR and an intron. Such sequences downstream of the TSS can contribute to regulation of expression at the transcriptional and/or translational stages.

The term “synthetic promoter” as used herein relates to a promoter that does not occur in nature. In the present context it typically comprises a CRE and/or CRM of the present invention operably linked to a minimal (or core) promoter or muscle-specific or skeletal muscle-specific proximal promoter (promoter element). The CREs and/or CRMs of the present invention serve to enhance muscle-specific or skeletal muscle-specific transcription of a gene operably linked to the synthetic promoter. Parts of the synthetic promoter may be naturally occurring (e.g. the minimal promoter or one or more CREs in the promoter), but the synthetic promoter as an entity is not naturally occurring.

As used herein, “minimal promoter” (also known as the “core promoter”) refers to a typically short DNA segment which is inactive or largely inactive by itself, but can mediate transcription when combined with other transcription regulatory elements. Minimal promoter sequences can be derived from various different sources, including prokaryotic and eukaryotic genes. Examples of minimal promoters are discussed above, and include the desmin minimum promoter, dopamine beta-hydroxylase gene minimum promoter, cytomegalovirus (CMV) immediate early gene minimum promoter (CMV-MP), and the herpes thymidine kinase minimal promoter (MinTK). A minimal promoter typically comprises the transcription start site (TSS) and elements directly upstream, a binding site for RNA polymerase II, and general transcription factor binding sites (often a TATA box). A minimal promoter may also include some elements downstream of the TSS, but these typically have little functionality absent additional regulatory elements.

As used herein, “proximal promoter” relates to the minimal promoter plus at least some additional regulatory sequence, typically the proximal sequence upstream of the gene that tends to contain primary regulatory elements. It often extends approximately 250 base pairs upstream of the TSS, and includes specific TFBS. A proximal promoter may also include one or more regulatory elements downstream of the TSS, for example a UTR or an intron. In the present case, the proximal promoter may suitably be a naturally occurring muscle-specific or skeletal muscle-specific proximal promoter that can be combined with one or more CREs or CRMs of the present invention. However, the proximal promoter can be synthetic.

As used herein, “promoter element” refers to either a minimal promoter or proximal promoter as defined above. In the context of the present invention a promoter element is typically combined with one or more CREs in order to provide a synthetic muscle-specific or skeletal muscle-specific promoter of the present invention.

A “functional variant” of a CRE, CRM, promoter element, synthetic promoter or other regulatory nucleic acid in the context of the present invention is a variant of a reference sequence that retains the ability to function in the same way as the reference sequence, e.g. as a muscle-specific or skeletal muscle-specific CRE, muscle-specific or skeletal muscle-specific CRM or muscle-specific or skeletal muscle-specific promoter. Alternative terms for such functional variants include “biological equivalents” or “equivalents”.

It will be appreciated that the ability of a given CRE, CRM, promoter or other regulatory sequence to function as a muscle-specific or skeletal muscle-specific enhancer is determined significantly by the ability of the sequence to bind the same muscle-specific, cardiac muscle-specific, or skeletal muscle-specific TFs that bind to the reference sequence. Accordingly, in most cases, a functional variant of a CRE or CRM will contain TFBS for the most or all of same TFs as the reference CRE, CRM or promoter. It is preferred, but not essential, that the TFBS of a functional variant are in the same relative positions (i.e. order and general position) as the reference CRE, CRM or promoter. It is also preferred, but not essential, that the TFBS of a functional variant are in the same orientation as the reference sequence (it will be noted that TFBS can in some cases be present in reverse orientation, e.g. as the reverse complement vis-à-vis the sequence in the reference sequence). It is also preferred, but not essential, that the TFBS of a functional variant are on the same strand as the reference sequence. Thus, in preferred embodiments, the functional variant comprises TFBS for the same TFs, in the same order, the same position, in the same orientation and on the same strand as the reference sequence. It will also be appreciated that the sequences lying between TFBS (referred to in some cases as spacer sequences, or suchlike) are of less consequence to the function of the CRE or CRM. Such sequences can typically be varied considerably, and their lengths can be altered. However, in preferred embodiments the spacing (i.e. the distance between adjacent TFBS) is substantially the same (e.g. it does not vary by more than 20%, preferably by not more than 10%, and more preferably it is approximately the same) in a functional variant as it is in the reference sequence. It will be apparent that in some cases a functional variant of a CRE can be present in the reverse orientation, e.g. it can be the reverse complement of a CRE as described above, or a variant thereof.

Levels of sequence identity between a functional variant and the reference sequence can also be an indicator for retained functionality. High levels of sequence identity in the TFBS of the CRE, CRM or promoter is of generally higher importance than sequence identity in the spacer sequences (where there is little or no requirement for any conservation of sequence). However, it will be appreciated that even within the TFBS, a considerable degree of sequence variation can be accommodated, given that the sequence of a functional TFBS does not need to exactly match the consensus sequence.

The ability of one or more TFs to bind to a TFBS in a given functional variant can determined by any relevant means known in the art, including, but not limited to, electromobility shift assays (EMSA), binding assays, chromatin immunoprecipitation (ChIP), and ChIP-sequencing (ChIP-seq). In a preferred embodiment the ability of one or more TFs to bind a given functional variant is determined by EMSA. Methods of performing EMSA are well-known in the art. Suitable approaches are described in Sambrook et al. cited above. Many relevant articles describing this procedure are available, e.g. Hellman and Fried, Nat Protoc. 2007; 2 (8): 1849-1861.

“Muscle-specific” or “muscle-specific expression” refers to the ability of a cis-regulatory element, cis-regulatory module or promoter to enhance or drive expression of a gene in muscle cells (or in muscle-derived cells) in a preferential or predominant manner as compared to other tissues (e.g. liver, kidney, spleen, heart, lung, and brain). Expression of the gene can be in the form of mRNA or protein. In preferred embodiments, muscle-specific expression is such that there is negligible expression in other (i.e. non-muscle) tissues or cells, i.e. expression is highly muscle-specific. For example, expression in muscle cells as opposed to other cells is at least 75%, 80%, 85%, 90% or 95%. “Cardiac muscle-specific” or “Cardiac muscle-specific expression” refers to the ability of a cis-regulatory element, cis-regulatory module, promoter element or promoter to enhance or drive expression of a gene in the cardiac muscle in a preferential or predominant manner as compared to other tissues (e.g. spleen, liver, lung, and brain) and compared to the skeletal muscle tissue. “Skeletal muscle-specific” or “Skeletal muscle-specific expression” refers to the ability of a cis-regulatory element, cis-regulatory module, promoter element or promoter to enhance or drive expression of a gene in the skeletal muscle in a preferential or predominant manner as compared to other tissues (e.g. spleen, liver, lung, and brain) and compared to the cardiac muscle tissue. There can be instances where lower degrees of specificity are desired and are part of this invention.

The ability of a CRE, CRM or promoter to function as a muscle-specific, cardiac muscle-specific or skeletal muscle-specific CRE, CRM or promoter can be readily assessed by the skilled person. The skilled person can thus easily determine whether any variant of the specific CRE, CRM or promoter recited above remains functional (i.e. it is a functional variant as defined above). For example, any given CRM to be assessed can be operably linked to a minimal promoter (e.g. positioned upstream of CMV-MP) and the ability of the cis-regulatory element to drive muscle-specific, cardiac muscle-specific or skeletal muscle-specific expression of a gene (typically a reporter gene) is measured. Alternatively, a variant of a CRE or CRM can be substituted into a synthetic muscle-specific, cardiac muscle-specific or skeletal muscle-specific promoter in place of a reference CRE or CRM, and the effects on muscle-specific, cardiac muscle-specific or skeletal muscle-specific expression driven by said modified promoter can be determined and compared to the unmodified form. Similarly, the ability of a promoter to drive muscle-specific, cardiac muscle-specific or skeletal muscle-specific expression can be readily assessed by the skilled person (e.g. as described in the examples below). Expression levels of a gene driven by a variant of a reference promoter can be compared to the expression levels driven by the reference promoter. In some embodiments, where muscle-specific or skeletal muscle-specific expression levels driven by a variant promoter are at least 50%, at least 60%, at least 70%, at least 80%, at least 90%, or at least 100% of the expression levels driven by the reference promoter, it can be said that the variant remains functional. Suitable nucleic acid constructs and reporter assays to assess muscle-specific, cardiac muscle-specific or skeletal muscle-specific expression enhancement can easily be constructed, and the examples set out below give suitable methodologies.

Muscle-specificity, cardiac muscle-specific or skeletal muscle-specificity can be identified wherein the expression of a gene (e.g. a therapeutic or reporter gene) occurs preferentially or predominantly in muscle-derived cells or skeletal muscle. Preferential or predominant expression can be defined, for example, where the level of expression is significantly greater in muscle-derived, cardiac muscle-specific or skeletal muscle-derived cells than in other types of cells (i.e. non-muscle-derived cells, non-cardiac muscle-specific or non-skeletal muscle-derived cells). For example, expression in muscle-derived, cardiac muscle-specific or skeletal muscle-derived cells is suitably at least 5-fold higher than in non-muscle cells, non-cardiac muscle-specific or non-skeletal muscle cells, preferably at least 10-fold higher than in non-muscle cells or non-skeletal muscle cells, and it may be 50-fold higher or more in some cases. For convenience, muscle-specific expression can suitably be demonstrated via a comparison of expression levels in a muscle cell line (e.g. muscle-derived cell line such as C2C12 or H2K cells (skeletal muscle) or H9C2 cells (cardiac), compared with expression levels in a liver-derived cell line (e.g. Huh7 or HepG2), kidney-derived cell line (e.g. HEK-293), a cervical tissue-derived cell line (e.g. HeLa) and/or a lung-derived cell line (e.g. A549). Cardiac muscle-specific expression can suitably be demonstrated via a comparison of expression levels in a cardiac muscle cell line (e.g. cardiac muscle derived cell line such as H9C2) or primary cardiomyocyte compared with expression levels in in a liver-derived cell line (e.g. Huh7 or HepG2), a kidney-derived cell line (e.g. HEK-293), a cervical tissue-derived cell line (e.g. HeLa), a lung-derived cell line (e.g. A549) and/or skeletal muscle-derived cells (e.g. C2C12 or H2K). Skeletal muscle-specific expression can suitably be demonstrated via a comparison of expression levels in a skeletal muscle-derived cells (e.g. C2C12 or H2K) or primary skeletal muscle cells compared with expression levels in in a liver-derived cell line (e.g. Huh7 or HepG2), a kidney-derived cell line (e.g. HEK-293), a cervical tissue-derived cell line (e.g. HeLa), a lung-derived cell line (e.g. A549) and/or cardiac muscle cell line (e.g. H9C2).

The synthetic muscle-specific, cardiac muscle-specific or skeletal muscle-specific promoters of the present invention preferably exhibit reduced expression in non-muscle-derived cells, suitably in Huh7, HEK-293, HeLa, and/or A549 cells when compared to a non-tissue specific promoter such as CMV-IE. The synthetic muscle-specific, cardiac muscle-specific or skeletal muscle-specific promoters of the present invention preferably have an activity of 50% or less than the CMV-IE promoter in non-muscle-derived cells (suitably in Huh7, HEK-293, HeLa, and/or A549 cells), suitably 25% or less, 20% or less, 15% or less, 10% or less, 5% or less or 1% or less. Generally, it is preferred that expression in non-muscle-derived cells is minimized, but in some cases this may not be necessary. Even if a synthetic promoter of the present invention has higher expression in, e.g., one or two non-muscle cells, as long as it generally has higher expression overall in a range of muscle cells versus non-muscle cell, it can still a muscle-specific promoter. In some embodiments, a muscle-specific promoter expresses a gene at least 25%, or at least 35%, or at least 45%, or at least 55%, or at least 65%, or at least 75%, or at least 80%, or at least 85%, or at least 90%, or at least 95%, or any integer between 25%-95% higher in muscle cells as compared to non-muscle cells.

The synthetic muscle-specific promoters of the present invention are preferably suitable for promoting expression in the muscle of a subject, e.g. driving muscle-specific expression of a transgene, preferably a therapeutic transgene. The synthetic skeletal muscle-specific promoters of the present invention are preferably suitable for promoting expression in the skeletal muscles of a subject, e.g. driving skeletal muscle-specific expression of a transgene, preferably a therapeutic transgene. Preferred synthetic muscle-specific promoters of the present invention are suitable for promoting muscle-specific transgene expression and have an activity in muscle cells which is at least 15%, 20%, 30%, 40%, 50%, 60%, 70%, 80%, 90%, 100%, 125%, 150%, 175%, 200%, 250%, 300%, 350% or 400% of the activity of the CBA promoter. In some embodiments, the synthetic muscle-specific promoters of the invention are suitable for promoting muscle-specific transgene expression at a level at least 100% of the activity of the CBA promoter, preferably 150%, 200%, 300% or 500% of the activity of the CBA or the SPc5-12 promoter. In some embodiments, the synthetic skeletal muscle-specific promoters of the invention are suitable for promoting skeletal muscle-specific transgene expression at a level at least 100% of the activity of the Tnnt2 or Myl2 promoter, preferably 150%, 200%, 300% or 500% of the activity of the SPc5-12 promoter. Such muscle-specific expression is suitably determined in muscle-derived cells, e.g. as C2C12 or H2K cells (skeletal muscle) or H9C2 cells (cardiac) or primary muscle cells (suitably primary human myocytes).

Synthetic muscle-specific, cardiac muscle-specific or skeletal muscle-specific promoters of the present invention may also be able to promote muscle-specific or skeletal muscle-specific expression of a gene at a level at least 50%, 100%, 150% or 200% compared to CMV-IE in muscle-derived cells (e.g. C2C12 or H2K cells (skeletal muscle) or H9C2 cells (cardiac)).

The term “nucleic acid” as used herein typically refers to an oligomer or polymer (preferably a linear polymer) of any length composed essentially of nucleotides. A nucleotide unit commonly includes a heterocyclic base, a sugar group, and at least one, e.g. one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups. Nucleic acids as intended herein may include naturally occurring nucleotides, modified nucleotides or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term “nucleic acid” further preferably encompasses DNA, RNA and DNA RNA hybrid molecules, specifically including hnRNA, pre-mRNA, mRNA, cDNA, genomic DNA, amplification products, oligonucleotides, and synthetic (e.g., chemically synthesised) DNA, RNA or DNA RNA hybrids. A nucleic acid can be naturally occurring, e.g., present in or isolated from nature; or can be non-naturally occurring, e.g., recombinant, i.e., produced by recombinant DNA technology, and/or partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

By “isolated” is meant, when referring to a nucleic acid is a nucleic acid molecule devoid, in whole or part, of sequences normally associated with it in nature; or a sequence, as it exists in nature, but having heterologous sequences in association therewith; or a molecule disassociated from the chromosome.

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215:403-10), such as the “Blast 2 sequences” algorithm described by Tatusova and Madden 1999 (FEMS Microbiol Lett 174:247-250).

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method. Typically, the percentage sequence identity is calculated over the entire length of the sequence.

For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

The term “hybridising” means annealing to two at least partially complementary nucleotide sequences in a hybridization process. In order to allow hybridisation to occur complementary nucleic acid molecules are generally thermally or chemically denatured to melt a double strand into two single strands and/or to remove hairpins or other secondary structures from single-stranded nucleic acids. The stringency of hybridisation is influenced by conditions such as temperature, salt concentration and hybridisation buffer composition. Conventional hybridisation conditions are described in, for example, Sambrook (2001) Molecular Cloning: a laboratory manual, 3^{rd} Edition Cold Spring Harbor Laboratory Press, CSH, New York, but the skilled craftsman will appreciate that numerous different hybridisation conditions can be designed in function of the known or the expected homology and/or length of the nucleic acid sequence. High stringency conditions for hybridisation include high temperature and/or low sodium/salt concentration (salts include sodium as for example in NaCl and Na-citrate) and/or the inclusion of formamide in the hybridisation buffer and/or lowering the concentration of compounds such as SDS (sodium dodecyl sulphate detergent) in the hybridisation buffer and/or exclusion of compounds such as dextran sulphate or polyethylene glycol (promoting molecular crowding) from the hybridisation buffer. By way of non-limiting example, representative salt and temperature conditions for stringent hybridization are: 1×SSC, 0.5% SDS at 65° C. The abbreviation SSC refers to a buffer used in nucleic acid hybridization solutions. One litre of a 20× (twenty times concentrate) stock SSC buffer solution (pH 7.0) contains 175.3 g sodium chloride and 88.2 g sodium citrate. A representative time period for achieving hybridisation is 12 hours.

The term “transcription factor binding site” (TFBS) is well known in the art. It will be apparent to the skilled person that alternative TFBS sequences can be used, provided that they are bound by the intended TF. Consensus sequences for the various TFBS disclosed herein are known in the art, and the skilled person can readily use this information to determine alternative TFBS. Furthermore, the ability of a TF to bind to a given putative sequence can readily be determined experimentally by the skilled person (e.g. by EMSA and other approaches well known in the art and discussed herein).

The meaning of “consensus sequence” is well-known in the art. In the present application, the following notation is used for the consensus sequences, unless the context dictates otherwise. Considering the following exemplary DNA sequence:
A[CT]N{A}YR

A means that an A is always found in that position; [CT] stands for either C or T in that position; N stands for any base in that position; and {A} means any base except A is found in that position. Y represents any pyrimidine, and R indicates any purine.

“Synthetic” in the present application means a nucleic acid molecule that does not occur in nature. Synthetic nucleic acids of the present invention are produced artificially, typically by recombinant technologies or de novo synthesis. Such synthetic nucleic acids may contain naturally occurring sequences (e.g. promoter, enhancer, intron, and other such regulatory sequences), but these are present in a non-naturally occurring context. For example, a synthetic gene (or portion of a gene) typically contains one or more nucleic acid sequences that are not contiguous in nature (chimeric sequences), and/or may encompass substitutions, insertions, and deletions and combinations thereof.

“Complementary” or “complementarity”, as used herein, refers to the Watson-Crick base-pairing of two nucleic acid sequences. For example, for the sequence 5′-AGT-3′ binds to the complementary sequence 3′-TCA-5′. Complementarity between two nucleic acid sequences may be “partial”, in which only some of the bases bind to their complement, or it may be complete as when every base in the sequence binds to its complementary base. The degree of complementarity between nucleic acid strands has significant effects on the efficiency and strength of hybridisation between nucleic acid strands.

“Transfection” in the present application refers broadly to any process of deliberately introducing nucleic acids into cells, and covers introduction of viral and non-viral vectors, and includes or is equivalent to transformation, transduction and like terms and processes. Examples include, but are not limited to: transfection with viral vectors; transformation with plasmid vectors; electroporation (Fromm et al. (1986) Nature 319:791-3); lipofection (Feigner et al. (1987) Proc. Natl. Acad. Sci. USA 84:7413-7); microinjection (Mueller et al. (1978) Cell 15:579-85); Agrobacterium-mediated transfer (Fraley et al. (1983) Proc. Natl. Acad. Sci. USA 80:4803-7); direct DNA uptake; whiskers-mediated transformation; and microprojectile bombardment (Klein et al. (1987) Nature 327:70).

As used herein, the phrase “transgene” refers to an exogenous nucleic acid sequence. In one example, a transgene is a gene encoding an industrially or pharmaceutically useful compound, or a gene encoding a desirable trait. In yet another example, the transgene encodes useful nucleic acid such as an antisense nucleic acid sequence, wherein expression of the antisense nucleic acid sequence inhibits expression of a target nucleic acid sequence. The transgene preferably encodes a therapeutic product, e.g. a protein.

The term “vector” is well known in the art, and as used herein refers to a nucleic acid molecule, e.g. double-stranded DNA, which may have inserted into it a nucleic acid sequence according to the present invention. A vector is suitably used to transport an inserted nucleic acid molecule into a suitable host cell. A vector typically contains all of the necessary elements that permit transcribing the insert nucleic acid molecule, and, preferably, translating the transcript into a polypeptide. A vector typically contains all of the necessary elements such that, once the vector is in a host cell, the vector can replicate independently of, or coincidental with, the host chromosomal DNA; several copies of the vector and its inserted nucleic acid molecule may be generated. Vectors of the present invention can be episomal vectors (i.e., that do not integrate into the genome of a host cell), or can be vectors that integrate into the host cell genome. This definition includes both non-viral and viral vectors. Non-viral vectors include but are not limited to plasmid vectors (e.g. pMA-RQ, pUC vectors, bluescript vectors (pBS) and pBR322 or derivatives thereof that are devoid of bacterial sequences (minicircles)) transposons-based vectors (e.g. PiggyBac (PB) vectors or Sleeping Beauty (SB) vectors), etc. Larger vectors such as artificial chromosomes (bacteria (BAC), yeast (YAC), or human (HAC)) may be used to accommodate larger inserts. Viral vectors are derived from viruses and include but are not limited to retroviral, lentiviral, adeno-associated viral, adenoviral, herpes viral, hepatitis viral vectors or the like. Typically, but not necessarily, viral vectors are replication-deficient as they have lost the ability to propagate in a given cell since viral genes essential for replication have been eliminated from the viral vector. However, some viral vectors can also be adapted to replicate specifically in a given cell, such as e.g. a cancer cell, and are typically used to trigger the (cancer) cell-specific (onco)lysis. Virosomes are a non-limiting example of a vector that comprises both viral and non-viral elements, in particular they combine liposomes with an inactivated HIV or influenza virus (Yamada et al., 2003). Another example encompasses viral vectors mixed with cationic lipids.

The term “operably linked”, “operably connected” or equivalent expressions as used herein refer to the arrangement of various nucleic acid elements relative to each other such that the elements are functionally connected and are able to interact with each other in the manner intended. Such elements may include, without limitation, a synthetic promoter, a CRE (e.g. enhancer or other regulatory element), CRM, a promoter element, a polyadenylation sequence, one or more introns and/or exons, and a coding sequence of a gene of interest to be expressed. The nucleic acid sequence elements, when properly oriented or operably linked, act together to modulate the activity of one another, and ultimately may affect the level of expression of an expression product. By modulate is meant increasing, decreasing, or maintaining the level of activity of a particular element. The position of each element relative to other elements may be expressed in terms of the 5′ terminus and the 3′ terminus of each element or their position upstream or downstream of another element or position (such as a TSS or promoter element), and the distance between any particular elements may be referenced by the number of intervening nucleotides, or base pairs, between the elements. As understood by the skilled person, operably linked implies functional activity, and is not necessarily related to a natural positional link. Indeed, when used in nucleic acid expression cassettes, CREs will typically be located immediately upstream of the promoter element (although this is generally the case, it should definitely not be interpreted as a limitation or exclusion of positions within the nucleic acid expression cassette), but this needs not be the case in vivo, e.g., a regulatory element sequence naturally occurring downstream of a gene whose transcription it affects is able to function in the same way when located upstream of the promoter. Hence, according to a specific embodiment, the regulatory or enhancing effect of the regulatory element can be position-independent.

A “spacer sequence” or “spacer” as used herein is a nucleic acid sequence that separates two functional nucleic acid sequences (e.g. TFBS, CREs, CRMs, promoter element, etc.). It can have essentially any sequence, provided it does not prevent the functional nucleic acid sequence (e.g. cis-regulatory element) from functioning as desired (e.g. this could happen if it includes a silencer sequence, prevents binding of the desired transcription factor, or suchlike). Typically, it is non-functional, as in it is present only to space adjacent functional nucleic acid sequences from one another. In some embodiments, spacers may have a length of 75, 50, 40, 30, 30 or 10 nucleotides or fewer. In some embodiments, a spacer or spacers may be recognition sites for one or more restriction enzymes.

The term “pharmaceutically acceptable” as used herein is consistent with the art and means compatible with the other ingredients of the pharmaceutical composition and not deleterious to the recipient thereof.

“Therapeutically effective amount” and like phrases mean a dose or plasma concentration in a subject that provides the desired specific pharmacological effect, e.g. to express a therapeutic gene in the muscle. A therapeutically effective amount may not always be effective in treating the conditions described herein, even though such dosage is deemed to be a therapeutically effective amount by those of skill in the art. The therapeutically effective amount may vary based on the route of administration and dosage form, the age and weight of the subject, and/or the disease or condition being treated.

The term “AAV vector” as used herein is well known in the art, and generally refers to an AAV vector nucleic acid sequence including various nucleic acid sequences. An AAV vector as used herein typically comprise a heterologous nucleic acid sequence not of AAV origin as part of the vector. This heterologous nucleic acid sequence typically comprises a promoter as disclosed herein as well as other sequences of interest for the genetic transformation of a cell. In general, the heterologous nucleic acid sequence is flanked by at least one, and generally by two AAV inverted terminal repeat sequences (ITRs). An “AAV virion” or “AAV virus” or “AAV viral particle” or “AAV vector particle” refers to a viral particle composed of at least one AAV capsid polypeptide (including both variant AAV capsid polypeptides and non-variant parent capsid polypeptides) and an encapsidated polynucleotide AAV vector. If the particle comprises a heterologous nucleic acid (i.e. a polynucleotide other than a wild-type AAV genome, such as a transgene to be delivered to a mammalian cell), it can be referred to as an “AAV vector particle” or simply an “AAV vector”. Thus, production of AAV virion or AAV particle necessarily includes production of AAV vector as such a vector is contained within an AAV virion or AAV particle.

A “small interfering” or “short interfering RNA” or siRNA is a RNA duplex of nucleotides targeted to a gene interest (a “target gene”). An “RNA duplex” refers to the structure formed by the complementary pairing between two regions of a RNA molecule. siRNA is “targeted” to a gene and the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences forming the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length.

As used herein, the term “microRNA” refers to any type of interfering RNAs, including but not limited to, endogenous microRNAs and artificial microRNAs (e.g., synthetic miRNAs). Endogenous microRNAs are small RNAs naturally encoded in the genome capable of modulating the productive utilization of mRNA. An artificial microRNA can be any type of RNA sequence, other than endogenous microRNA, capable of modulating the activity of an mRNA. A microRNA sequence can be an RNA molecule composed of any one or more of these sequences. MicroRNA (or “miRNA”) sequences have been described in publications such as Lim, et al, 2003, Genes & Development, 17, 991-1008, Lim et al, 2003, Science, 299, 1540, Lee and Ambrose, 2001, Science, 294, 862, Lau et al, 2001, Science 294, 858-861, Lagos-Quintana et al, 2002, Current Biology, 12, 735-739, Lagos-Quintana et al, 2001, Science, 294, 853-857, and Lagos-Quintana et al., 2003, RNA, 9, 175-179. Examples of microRNAs include any RNA fragment of a larger RNA or is a miRNA, siRNA, stRNA, sncRNA, tncRNA, snoRNA, smRNA, shRNA, snRNA, or other small non-coding RNA. See, e.g., U.S. patent applications Ser. No. 20050272923, 20050266552, 20050142581, and 20050075492. A “microRNA precursor” (or “pre-miRNA”) refers to a nucleic acid having a stem-loop structure with a microRNA sequence incorporated therein. A “mature microRNA” (or “mature miRNA”) includes a microRNA cleaved from a microRNA precursor (a “pre-miRNA”), or synthesized (e.g., synthesized in a laboratory by cell-free synthesis), and has a length of from about 19 nucleotides to about 27 nucleotides, e.g., a mature microRNA can have a length of 19 nt, 20 nt, 21 nt, 22 nt, 23 nt, 24 nt, 25 nt, 26 nt, or 27 nt. A mature microRNA can bind to a target mRNA and inhibit translation of the target mRNA.

The terms “treatment” or “treating” refer to reducing, ameliorating or eliminating one or more signs, symptoms, or effects of a disease or condition. “Treatment,” as used herein thus includes any treatment of a disease in a mammal, particularly in a human, and includes: (a) preventing the disease from occurring in a subject predisposed to the disease or at risk of acquiring the disease but has not yet been diagnosed as having it; (b) inhibiting the disease, i.e., arresting its development; and (c) relieving the disease, i.e., causing regression of the disease.

The “administration” of an agent to a subject includes any route of introducing or delivering to a subject the agent to perform its intended function. Administration can be carried out by any suitable route, including orally, intranasally, intraocularly, ophthalmically, parenterally (intravenously, intramuscularly, intraperitoneally, or subcutaneously), or topically. Administration may be via anterograde epicardial coronary artery infusion. Administration includes self-administration and the administration by another. Intramuscular administration is of particular interest in the present invention.

The terms “individual,” “subject,” and “patient” are used interchangeably, and refer to any individual subject with a disease or condition in need of treatment. For the purposes of the present disclosure, the subject may be a primate, preferably a human, or another mammal, such as a dog, cat, horse, pig, goat, or bovine, and the like.

### EXAMPLES

The strength of the synthetic muscle-specific promoters or skeletal muscle-specific promoters according to certain embodiments of this invention were tested by operably linking them to the reporter gene luciferase. The expression cassette comprising of the muscle-specific promoter or skeletal muscle-specific promoter to be tested and the luciferase gene was inserted into a suitable plasmid which was then transfected into a cell in order to test the expression from the promoters in these cells.

### Example 1—In Vitro Testing

### Materials and Methods

DNA preparations were transfected into H9C2 (a rat BDIX heart myoblast cell line, available from ATCC) to asses transcriptional activity. H9C2 cell line was used as previous experiments have shown it to be a good predictor of skeletal and cardiac muscle activity in vivo. DNA preparations comprised a synthetic promoter (e.g. SP0500) operably linked to luciferase.

### H9C2 Cell Culture and Transfection

H9C2 are a rat BDIX heart myoblast cell line. They have cardiac muscle properties, e.g. myotubes formed at confluency respond to acetylcholine.

### Cell Maintenance

H9C2 cells were cultured in DMEM (High Glucose, D6546, Sigma) with 1% FBS (Heat inactivated-Gibco 10270-106, lot number 42G2076K), 1% Glutamax (35050-038, Gibco), 1% Penicillin-streptomycin solution (15140-122, Gibco), in T-75 flasks. Cells were passaged at a sub confluent stage (70-80%) to avoid risk of the cells becoming confluent and fusing to form myotubes.

For passaging during cell maintenance, culture media was removed, cells were washed twice with 5 ml DPBS without CaCl₂), without MgCl₂ (14190-094, Gibco). The cells were detached from the flask by incubating with 1 ml Trypsin EDTA (25200-056, Gibco) for approximately 5 minutes. Then, 4 ml of culture medium was added to the flask and the mixture was gently pipetted up and down to help detach the cells from the flask surface. Cells were pelleted at 100 g for 3 minutes. Supernatant was disposed and cells were resuspended in 3 ml of culture medium. Cells were counted on the Countess automated cell counter, seeded at 1:3 to 1:10 i.e. seeding 1-3×10,000 cells/cm² and incubated at 37° C. 5% CO₂.

### Cell Transfection and Differentiation

H9C2 cells were collected from two T-75 flasks of approximately 70-80% confluency, by washing with DPBS, detaching from the flask using 1 ml Trypsin EDTA, washing off the flask's surface with 4 ml of culture medium and pelleting at 100 g for 3 minutes, as described above. Cells were resuspended in 45 ml culture medium and seed at a density of 40,000 cells/well in a 48 well flat bottom plate (300 μl/well) (353230, Corning). Cells in 48-well plates were incubated at 37° C. 5% CO₂.

Twenty-four hours later, the culture medium on the cells was replaced with 300 μl antibiotic-free culture medium (i.e., DMEM (High Glucose, D6546, Sigma) with 1% FBS (Heat inactivated-Gibco 10270-106, lot number 42G2076K), 1% Glutamax (35050-038, Gibco)). 300 ng of DNA per well was transfected with viafect (E4981, Promega) in a total complex volume of 30 μl per well. Plates were gently mixed following transfection and incubated at 37° C. 5% CO₂.

Twenty-four hours later, culture medium was removed from transfected cells and replaced with 300 μl differentiation media consisting of DMEM (High Glucose, D6546, Sigma), 1% Glutamax (35050-038, Gibco), 1% FBS (Heat inactivated-Gibco 10270-106, lot number 42G2076K), 1% Penicillin/streptomycin solution (15140-122, Gibco) and 0.1% Retinoid Acid (Sigma-R2625). Plates were incubated at 37° C. 5% CO₂ for 7 days to induce differentiation. After differentiation, cell morphology was observed to confirm differentiation into myotubes.

Cells were then washed with 500 μl DPBS, and lysed with 100 μl Luciferase Cell Culture Lysis 5× Reagent (E1531, Promega) diluted to 1× using Milli-Q water. Cell lysis reagent was pipetted up and down ten times and plates were then vortexed on a medium power for 30 minutes to promote cell lysis. Plates were sealed and stored at −80° C. prior to completing a luciferase assay. The data collected from luciferase assays following transfections in H9C2 cells is based on three technical replicates of at one biological replicate.

### Measurement of Luciferase Activity

Luciferase activity was measured using LARII (Dual Luciferase Reporter 1000 assay system, Promega, E1980)
24 h after transfection, the media was removed from the cells
The cells were washed once in 300 μl of DPBS.
Cells were lysed using 100 μl of passive lysis buffer and incubated with rocking for 15 minutes.
The cell debris was pelleted by centrifugation of the plate at max speed in a benchtop centrifuge for 1 min
10 μl sample was transferred into white 96-well plate and luminescence measured by injection of 50 μl of LARII substrate on a BMG Labtech FLUOstar Omega plate reader.

Results generated from these cell cultures are shown in FIG. 1. This figure shows that synthetic promoters SP0500, SP0510, SP0514 and SP0519 show good activity in the muscle cell line H9C2. Other similar promoters described herein are expected to have the same or better performance.

### Example 2—In Vitro Data

The experiment was performed as detailed above in Example 1. However, the data collected in this example from luciferase assays following transfections in H9C2 cells is based on three biological replicates each of which is an average of three technical replicates.

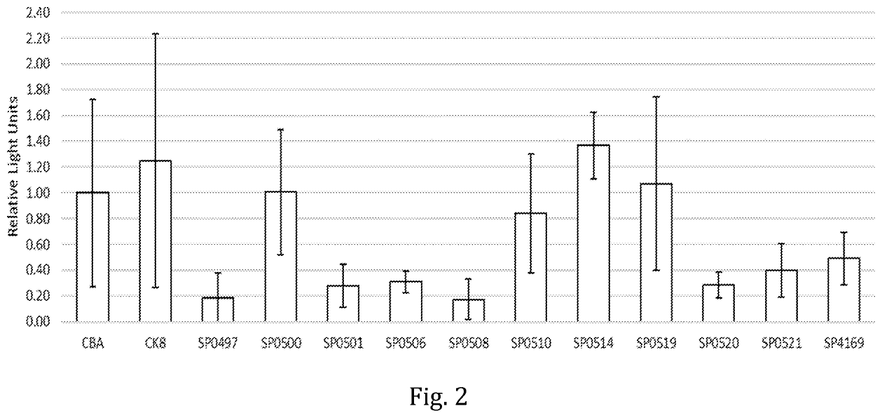

Results generated from these cell cultures are shown in FIG. 2, results are normalized relative to CBA. This figure shows that synthetic promoters SP0497, SP0500, SP0501, SP0506, SP0508, SP0510, SP0514, SP0519, SP0520, SP0521 and SP4169 show good activity in the muscle cell line H9C2. Promoters SP0498, SP0499, SP0502, SP0503, SP0504, SP0505, SP0507, SP0509, SP0511, SP0512, SP0513, SP0515, SP0516, SP0517, SP0518, SP0522, SP0523 and SP0524 were also tested experimentally in the H9C2 cell line but showed lower activity (data not shown).

### Example 3—In Vivo Data

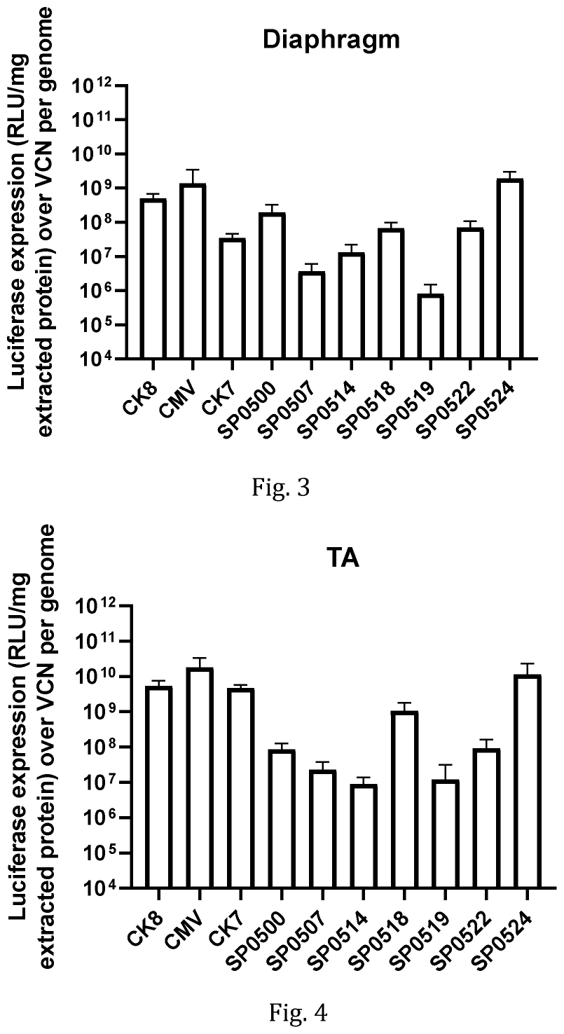

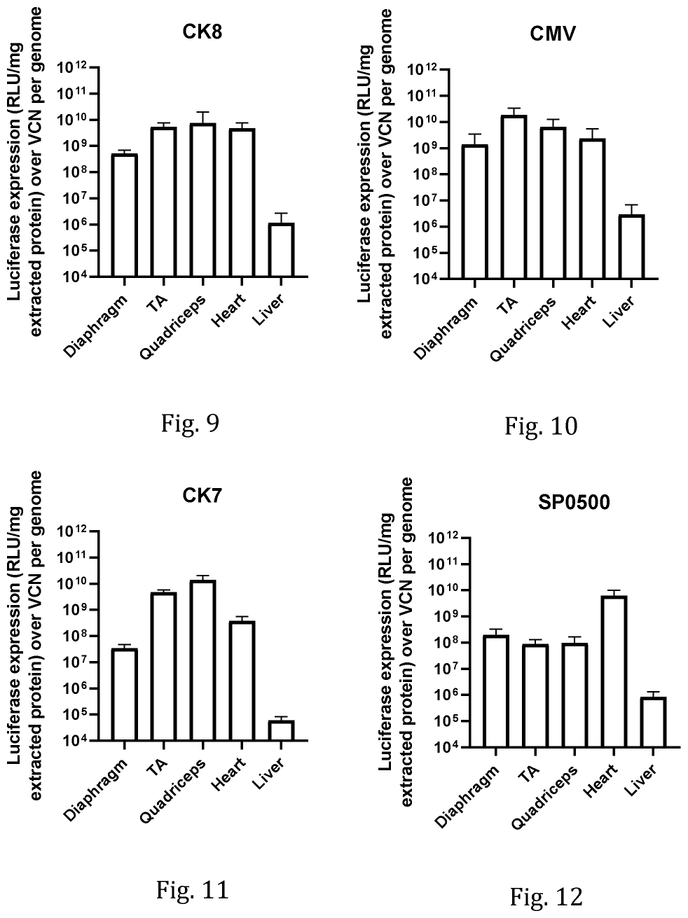

A selection of the synthetic muscle-specific promoters were tested in vivo, see e.g., FIGS. 3-18.

### Materials and Methods

AAVs comprising a synthetic promoter (e.g.; SP0500, SP0507, SP0514, SP0518, SP0519, SP0522, and SP0524) operably linked to luciferase were diluted in 0.9% saline and delivered via tail vein injection in 8-week-old male Balb/c mice at 1e¹¹ vg/200 μl per mouse (6 mice per group). Mice were sacrificed 6 weeks post injection. Diaphragm, heart, quadriceps, soleus, tibialis anterior (TA) and liver were collected from each mouse. For vector copy number (VCN) analysis, samples were snap-frozen in liquid nitrogen immediately after dissection and stored at −80° C.

Protein was extracted and quantified using a BCA Pierce protein assay kit ((ThermoFisher 23225) as per the manufacturer's instructions.

Luciferase quantification was done by ONE-Glo Luciferase assay system (Promega E6120).

DNA was extracted and all samples and reagents were incubated at room temperature until completely thawed and of equilibrated temperature. All samples and reagents were mixed well prior to use. ONE-Glo™ Reagent was added to each sample at an equal volume, and the samples were mixed thoroughly. After 3 minutes, to ensure complete cell lysis, the samples were measured in a luminometer.

Vector copy number was done by dual Taqman qPCR. DNA was extracted using the DNeasy® Blood & Tissue Kit (250), (QIAGEN, Catalog number #69506). Taqman qPCR performed on each sample using both Luciferase and GAPDH-specific primer and probe sets:

| Target | Primers | Probe |
|---|---|---|
| Luc2 | Fw (5′ ACGCTGGGCTACTTGATC 3′) | 5′ TTTCGGGTCGTGCTCATG 3′ |
| | (SEQ ID NO: 69) | (SEQ ID NO: 73) |
| | RV (5′ CAAGAATAGCTCCTCCTCG 3′) | FAM/BHQ1 (5′ reporter dye/3′ quencher) |
| | (SEQ ID NO: 70) | |
| mGapdH | FW (5′ ACGGCAAATTCAACGGCAC 3′) | 5′ TTGTCATCAACGGGAAGCCCATCA 3′ |
| | (SEQ ID NO: 71) | (SEQ ID NO: 74) |
| | RV (5′ TAGTGGGGTCTCGCTCCTGG 3′) | JOE/BHQ1 (5′ reporter dye/3′ quencher) |
| | (SEQ ID NO: 72) | |

Standard curves were used for Luc and GAPDH for analysis purposes. In the multiplex qPCR protocol, the following final concentrations of reagents and DNA were used: Luc2 fw Primer (350 nM), Luc2 RV Primer (350 nM), mGapdH FW Primer (350 nM), mGapdH RV Primer (350 nM), Luc2 Probe (250 nM), mGapdH Probe (250 nM) and DNA (10 ng/uL). The PCR cycle protocol was as follows: 95C-20 seconds, PCR: 40 cycles, 95C-1 second, 60C-20 seconds. The ΔΔCt VCN (Quantity) per genome was calculated by subtracting the average VCN (Quantity) per genome for the saline samples (threshold).

### Results

As shown in FIG. 12, synthetic muscle-specific promoter SP0500 showed high activity in the cardiac muscle (heart). Additionally, SP0500 showed activity in skeletal muscle (e.g. TA, quadriceps and diaphragm).

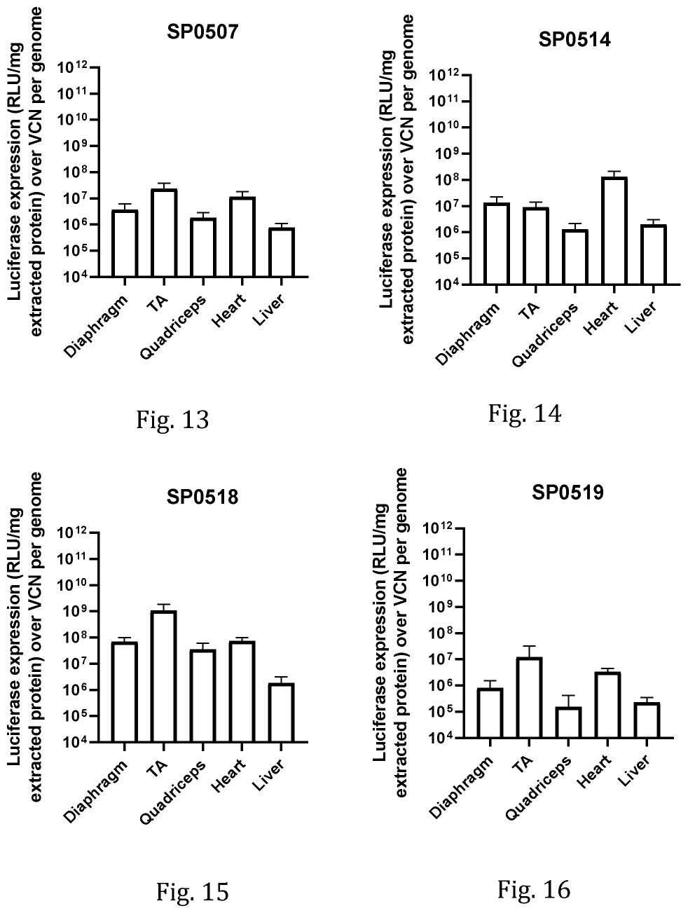

As shown in FIG. 13, synthetic muscle-specific promoter SP0507 shows activity in skeletal muscle (e.g. TA) and cardiac muscle (heart).

As shown in FIG. 14, synthetic muscle-specific promoter SP0514 shows activity in cardiac muscle (heart). Additionally, SP0514 shows some activity in skeletal muscle (e.g. diaphragm and TA).

As shown in FIG. 15, synthetic muscle-specific promoter SP0518 shows activity in skeletal muscle (e.g. TA). Additionally, SP0518 shows some activity in cardiac muscle.

As shown in FIG. 16, synthetic muscle-specific promoter SP0519 shows activity in skeletal muscle (e.g. TA). Additionally, SP0519 shows some activity in cardiac muscle.

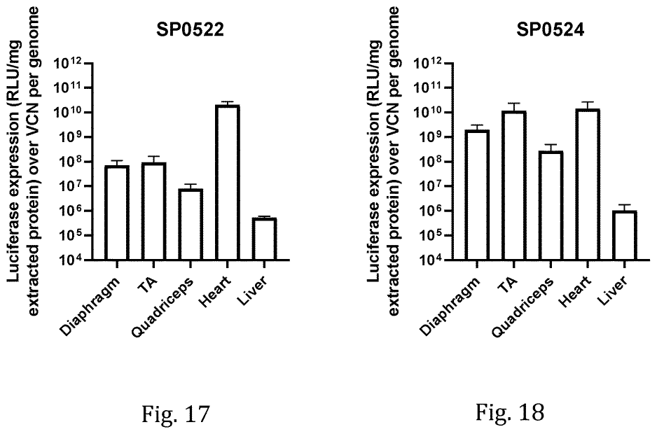

As shown in FIG. 17, synthetic muscle-specific promoter SP0522 showed high activity in the cardiac muscle (heart). Additionally, SP0522 showed activity in skeletal muscle (e.g. TA and diaphragm).

As shown in FIG. 18, synthetic muscle-specific promoter SP0524 showed high activity in the cardiac muscle (heart) and in skeletal muscle (e.g. TA, diaphragm and quadriceps).

As shown in FIG. 3, synthetic muscle-specific promoter SP0524 showed comparative or higher activity compared to control promoters CMV and CK8 in the diaphragm. Synthetic muscle-specific promoters SP500, SP0518 and SP0522 showed comparative or higher activity with respect to CK7 in the diaphragm.

As shown in FIG. 4, synthetic muscle-specific promoter SP0524 showed activity similar to control promoters CMV, CK7 and CK8 in the TA.

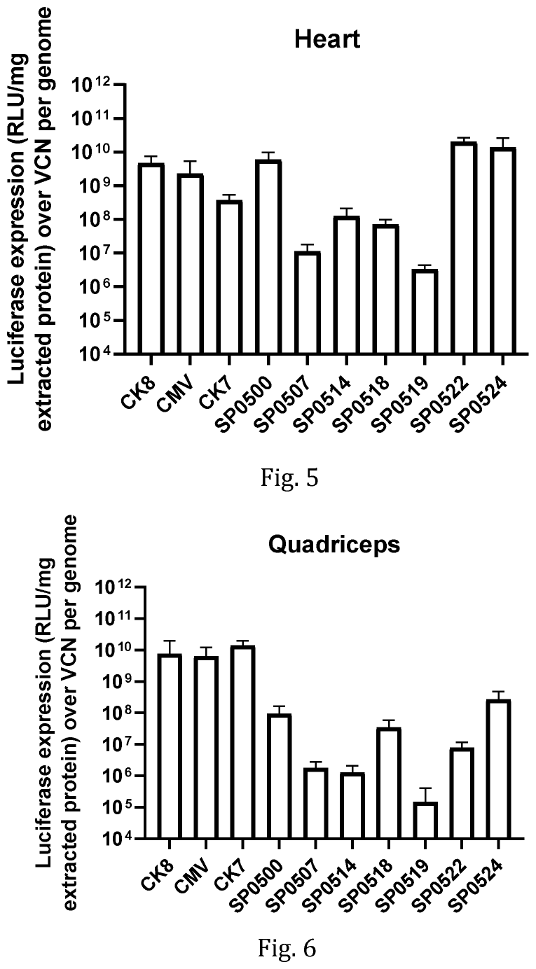

As shown in FIG. 5, synthetic muscle-specific promoters SP500, SP0522 and SP0524 showed comparative or higher activity compared to control promoters CK8, CMV and CK7 in the heart.

As shown in FIG. 6, all tested synthetic promoters showed lower activity compared to control promoters CK8, CMV and CK7 in the quadriceps.

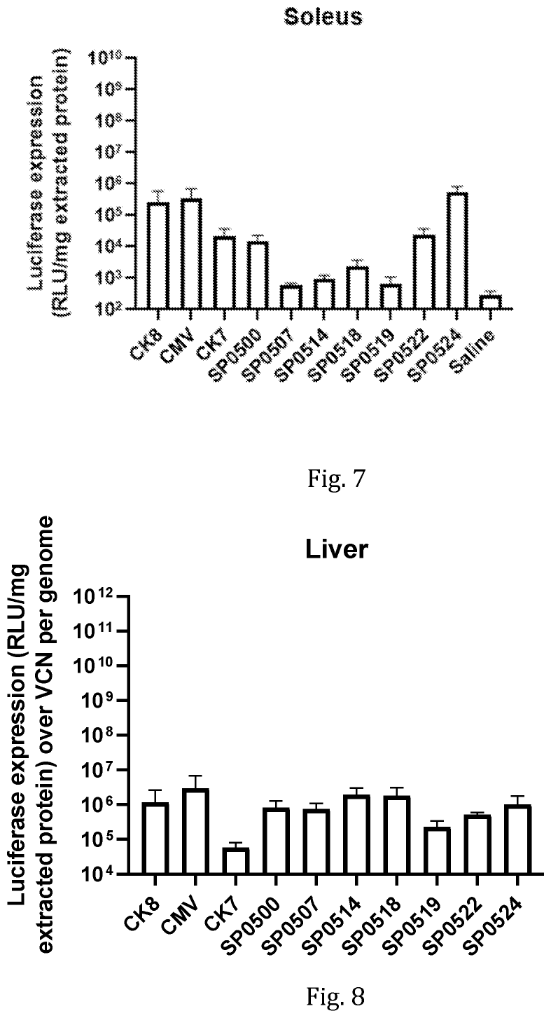

As shown in FIG. 7, synthetic muscle-specific promoter SP0524 showed comparative or higher activity compared to control promoters CK8 and CMV in the soleus. Synthetic muscle-specific promoters SP0500 and SP0522 showed comparative or higher activity compared to control promoter CK7.

As shown in FIG. 8, the tested synthetic muscle-specific promoters SP500, SP0507, SP0514, SP0518, SP0519, SP0522 and SP0524 showed lower or similar activity in the liver compared to control promoters CK8 and CMV.

### BIBLIOGRAPHY

Llanga, T. et al. (2017) ‘Structure-Based Designed Nano-Dysferlin Significantly Improves Dysferlinopathy in BLA/J Mice’, Molecular Therapy. Elsevier Ltd., 25 (9), pp. 2150-2162. doi: 10.1016/j.ymthe.2017.05.013.

### Sequence Information

TABLE 1
Muscle-Specific Synthetic Promoters

| | | |
|---|---|---|
| SP0497 | tctgagggagacagggaggcatgatcactgccaaatgcccaccaaggacaaggcaca | 270 |
| (SEQ ID NO: 1) | tcccagggagacagacgcagacctggtgccctctggacactggcattcctggagttctcct | |
| | ctataaatacccgctctggtatttggggttggcagctgttgcggccggggccgcattcctgg | |
| | gggccgggcggtgctcccgcccgcctcgataaaaggctccggggccggcggcggccc | |
| | acgagctacccggaggagcgggaggcggccacc | |
| SP0498 | tctgagggagacagggaggcatgatcactgccaaatgcccaccaaggacaaggcaca | 258 |
| (SEQ ID NO: 2) | tcccagggagacagacgcagacctggtgccctctggacactggcattcctggagagggt | |
| | cagtgtcctgccccaacccatgagatgacagactataatagccacaggattaacatagc | |
| | aggcattgcaccgcctgctgccacggccggccgtataaatagaggcgaggagcagctg | |
| | ggctctcttggcagtcaccgccacc | |
| SP0499 | tctgagggagacagggaggcatgatcactgccaaatgcccaccaaggacaaggcaca | 234 |
| (SEQ ID NO: 3) | tcccagggagacagacgcagacctggtgccctctggacactggcattcctggagttctcct | |
| | ctataaatacccgctctggtatttggggttggcagctgttgcaccgcctgctgccacggccg | |
| | gccgtataaatagaggcgaggagcagctgggctctcttggcagtcaccgccacc | |
| SP0500 | ccagcccacctgtcccaatgctgacttagtgcaaggcgagccagcaaggagggaggac | 267 |
| (SEQ ID NO: 4) | aggtggcagtggggggtgaggagcatctaaaaatagccccagcccacctgtcccaatg | |
| | ctgacttagtgcaaggcgagccagcaaggagggaggacaggtggcagtggggggtga | |
| | ggagcatctaaaaatagcccaccgcctgctgccacggccggccgtataaatagaggcg | |
| | aggagcagctgggctctcttggcagtcaccgccacc | |
| SP0501 | ccagcccacctgtcccaatgctgacttagtgcaaggcgagccagcaaggagggaggac | 219 |
| (SEQ ID NO: 5) | aggtggcagtggggggtgaggagcatctaaaaatagccttctcctctataaatacccgctc | |
| | tggtatttggggttggcagctgttgcaccgcctgctgccacggccggccgtataaatagag | |
| | gcgaggagcagctgggctctcttggcagtcaccgccacc | |
| SP0502 | ctagactagcatgctgcccatgtaaggaggcaaggcctggggacacccgagatgcctg | 251 |
| (SEQ ID NO: 6) | gttataattaacccagacatgtggctgcccccccccccccaacacctgctgcctctaaaaa | |
| | taaccctgcttctcctctataaatacccgctctggtatttggggttggcagctgttgcaccgcct | |
| | gctgccacggccggccgtataaatagaggcgaggagcagctgggctctcttggcagtca | |
| | ccgccacc | |
| SP0503 | tcgtcccctggctggcccatgtaatctgagcccagcattgtacatatcctgggaacagctga | 258 |
| (SEQ ID NO: 7) | caatgcagtggtcagacagctggtggggccagctagagctggcagggttggctgggag | |
| | gggagtgtaggctgattctcctctataaatacccgctctggtatttggggttggcagctgttgc | |
| | accgcctgctgccacggccggccgtataaatagaggcgaggagcagctgggctctcttg | |
| | gcagtcaccgccacc | |
| SP0504 | ttggcccaggtcacactggggtgaggctagtgttcctgagccttgacaaggagacagcttg | 265 |
| (SEQ ID NO: 8) | aaatagacgagtgtcacatttctgagcagctgtgtggcgacagcaggaggggtagggaa | |
| | tagacagtataaaagagaaagcttctcctctataaatacccgctctggtatttggggttggc | |
| | agctgttgcaccgcctgctgccacggccggccgtataaatagaggcgaggagcagctg | |
| | ggctctcttggcagtcaccgccacc | |
| SP0505 | ccctgccatcttgggtttcagggcagaggagtcttgctaattttgatgcctatttttggacacttc | 269 |
| (SEQ ID NO: 9) | agctgccactggctccttataaacgcatgacaccccatgcaaacacactacccctccctc | |
| | cactgctgacaggtgtgtggttctcctctataaatacccgctctggtatttggggttggcagct | |
| | gttgcaccgcctgctgccacggccggccgtataaatagaggcgaggagcagctgggctc | |
| | tcttggcagtcaccgccacc | |
| SP0506 | aattatttttaataacacttactggtaagagaaaggggagaaaccttagacaggcacttag | 274 |
| (SEQ ID NO: 10) | atgtgactaaggcaggtttatctctgattccaaagcactggagtggaagtcacaccgtgact | |
| | cagagcattgtgatgggccagctgtccattctcctctataaatacccgctctggtatttggggt | |
| | tggcagctgttgcaccgcctgctgccacggccggccgtataaatagaggcgaggagca | |
| | gctgggctctcttggcagtcaccgccacc | |
| SP0507 | cagtgttcctatatttatcccaccatacagagcttcctttgcctcagaaggaccagcagtttcg | 274 |
| (SEQ ID NO: 11) | ctagcttaacaaaaccagccactcagggtattggtttacagtcaagcaactctgggagag | |
| | ggcagctgctctcagacatcatacagcttctcctctataaatacccgctctggtatttggggtt | |
| | ggcagctgttgcaccgcctgctgccacggccggccgtataaatagaggcgaggagcag | |
| | ctgggctctcttggcagtcaccgccacc | |
| SP0508 | cttctcaagccaaaggagcaagagttaaaaataacaggctcaccctggcagccacctgt | 240 |
| (SEQ ID NO: 12) | gctggccagccccaccccatccctccctcggggacagctgcagctcctcaggccccgcc | |
| | cgggacattttgggaacactttctcctcttacttctcatcttcagggcaccgcctgctgccacg | |
| | gccggccgtataaatagaggcgaggagcagctgggctctcttggcagtcaccgccacc | |
| SP0509 | agggtcagtgtcctgccccaacccatgagatgacagactataatagccacaggattaac | 275 |
| (SEQ ID NO: 13) | atagcaggcattgatttttaaagactgaggaattaggcacctgtcatttttgccagctggtgta | |
| | gatgttaaaaattactgtcactcttccgcctgctactttattttgcacctgctgttacttgagttac | |
| | aggcatttcacaccgcctgctgccacggccggccgtataaatagaggcgaggagcagct | |
| | gggctctcttggcagtcaccgccacc | |
| SP0510 | atttttaaagactgaggaattaggcacctgtcatttttgccagctggtgtagatgttaaaaatta | 251 |
| (SEQ ID NO: 14) | ctgtcactcttccgcctgctactttattttgcacctgctgttacttgagttacaggcatttcattct | |
| | ctctataaatacccgctctggtatttggggttggcagctgttgcaccgcctgctgccacggcc | |
| | cggccgtataaatagaggcgaggagcagctgggctctcttggcagtcaccgccacc | |
| SP0511 | agactggggcaggtgcaggctggattgggtttccagaggctatatatataaaggctgccg | 274 |
| (SEQ ID NO: 15) | ggagccccagggccgctccctgagggcacaacactgtgggggcccagccaggcccac | |
| | attcctttccagaggccagctctccatttatagcccctgggcagagcagcttctcctctataa | |
| | atacccgctctggtatttggggttggcagctgttggggctgggcataaaagtcagggcaga | |
| | gccatctattgcttacatttgcttctggccacc | |
| SP0512 | tctgagggagacagggaggcatgatcactgccaaatgcccaccaaggacaaggcaca | 218 |
| (SEQ ID NO: 16) | tcccagggagacagacgcagacctggtgccctctggacactggcattcctggagttctcct | |
| | ctataaatacccgctctggtatttggggttggcagctgttggggctgggcataaaagtcagg | |
| | gcagagccatctattgcttacatttgcttctggccacc | |
| SP0513 | ccagcccacctgtcccaatgctgacttagtgcaaggcgagccagcaaggagggaggac | 275 |
| (SEQ ID NO: 17) | aggtggcagtggggggtgaggagcatctaaaaatagccagggtcagtgtcctgcccca | |
| | acccatgagatgacagactataatagccacaggattaacatagcaggcattgttctcctct | |
| | ataaatacccgctctggtatttggggttggcagctgttggggctgggcataaaagtcaggg | |
| | cagagccatctattgcttacatttgcttctggccacc | |
| SP0514 | ccagcccacctgtcccaatgctgacttagtgcaaggcgagccagcaaggagggaggac | 251 |
| (SEQ ID NO: 18) | aggtggcagtggggggtgaggagcatctaaaaatagccttctcctctataaatacccgctc | |
| | tggtatttggggttggcagctgttgttctcctctataaatacccgctctggtatttggggttggca | |
| | gctgttggggctgggcataaaagtcagggcagagccatctattgcttacatttgcttctggcc | |
| | acc | |
| SP0515 | ctagactagcatgctgcccatgtaaggaggcaaggcctggggacacccgagatgcctg | 235 |
| (SEQ ID NO: 19) | gttataattaacccagacatgtggctgcccccccccccccaacacctgctgcctctaaaaa | |
| | taaccctgcttctcctctataaatacccgctctggtatttggggttggcagctgttggggctgg | |
| | gcataaaagtcagggcagagccatctattgcttacatttgcttctggccacc | |
| SP0516 | tcgtcccctggctggcccatgtaatctgagcccagcattgtacatatcctgggaacagctga | 242 |
| (SEQ ID NO: 20) | caatgcagtggtcagacagctggtggggccagctagagctggcagggttggctgggag | |
| | gggagtgtaggctgattctcctctataaatacccgctctggtatttggggttggcagctgttgg | |
| | ggctgggcataaaagtcagggcagagccatctattgcttacatttgcttctggccacc | |
| SP0517 | ttggcccaggtcacactggggtgaggctagtgttcctgagccttgacaaggagacagcttg | 249 |
| (SEQ ID NO: 21) | aaatagacgagtgtcacatttctgagcagctgtgtggcgacagcaggaggggtagggaa | |
| | tagacagtataaaagagaaagcttctcctctataaatacccgctctggtatttggggttggc | |
| | agctgttggggctgggcataaaagtcagggcagagccatctattgcttacatttgcttctggc | |
| | cacc | |
| SP0518 | ccctgccatcttgggtttcagggcagaggagtcttgctaattttgatgcctatttttggacacttc | 253 |
| (SEQ ID NO: 22) | agctgccactggctccttataaacgcatgacaccccatgcaaacacactacccctccctc | |
| | cactgctgacaggtgtgtggttctcctctataaatacccgctctggtatttggggttggcagct | |
| | gttggggctgggcataaaagtcagggcagagccatctattgcttacatttgcttctggccac | |
| | c | |
| SP0519 | cttctcaagccaaaggagcaagagttaaaaataacaggctcaccctggcagccacctgt | 272 |
| (SEQ ID NO: 23) | gctggccagccccaccccatccctccctcggggacagctgcagctcctcaggccccgcc | |
| | cgggacattttgggaacactttctcctcttacttctcatcttcagggttctcctctataaataccc | |
| | gctctggtatttggggttggcagctgttggggctgggcataaaagtcagggcagagccatc | |
| | tattgcttacatttgcttctggccacc | |
| SP0520 | agggtcagtgtcctgccccaacccatgagatgacagactataatagccacaggattaac | 272 |
| (SEQ ID NO: 24) | atagcaggcattgatttttaaagactgaggaattaggcacctgtcatttttgccagctggtgta | |
| | gatgttaaaaattactgtcactcttccgcctgctactttattttgcacctgctgttacttgagttac | |
| | aggcatttcagggctgggcataaaagtcagggcagagccatctattgcttacatttgcttctg | |
| | gccacc | |
| SP0521 | ctagactagcatgctgcccatgtaaggaggcaaggcctggggacacccgagatgcctg | 263 |
| (SEQ ID NO: 25) | gttataattaacccagacatgtggctgcccccccccccccaacacctgctgcctctaaaaa | |
| | taaccctgcttctcctctataaatacccgctctggtatttggggttggcagctgttggtacttata | |
| | taagggggtgggggcgcgttcgtcctcagtcgcgatcgaacactcgagccgagcagac | |
| | gtgcctacggaccggccacc | |
| SP4169 | ctagactagcatgctgcccatgtaaggaggcaaggcctggggacacccgagatgcctg | 287 |
| (SEQ ID NO: 26) | gttataattaacccagacatgtggctgcccccccccccccaacacctgctgcctctaaaaa | |
| | taaccctgcttctcctctataaatacccgctctggtatttggggttggcagctgttgcggccgg | |
| | ggccgcattcctgggggccgggcggtgctcccgcccgcctcgataaaaggctccgggg | |
| | ccggcggcggcccacgagctacccggaggagcgggaggcggccacc | |
| SP0522 | gctgcccatgtaaggaggcaaggcctggggacacccgagatgcctggttataattaacc | 237 |
| (SEQ ID NO: 27) | cagacatgtggctgccccccccccccaacacctgctgcctctaaaaataaccctgttctcct | |
| | ctataaatacccgctctggtatttggggttggcagctgttgcaccgcctgctgccacggccg | |
| | gccgtataaatagaggcgaggagcagctgggctctcttggcagtcaccgccacc | |
| SP0523 | gctgcccatgtaaggaggcaaggcctggggacacccgagatgcctggttataattaacc | 221 |
| (SEQ ID NO: 28) | cagacatgtggctgccccccccccccaacacctgctgcctctaaaaataaccctgttctcct | |
| | ctataaatacccgctctggtatttggggttggcagctgttggggctgggcataaaagtcagg | |
| | gcagagccatctattgcttacatttgcttctggccacc | |
| SP0524 | gctgcccatgtaaggaggcaaggcctggggacacccgagatgcctggttataattaacc | 249 |
| (SEQ ID NO: 29) | cagacatgtggctgccccccccccccaacacctgctgcctctaaaaataaccctgttctcct | |
| | ctataaatacccgctctggtatttggggttggcagctgttggtacttatataagggggtgggg | |
| | gcgcgttcgtcctcagtcgcgatcgaacactcgagccgagcagacgtgcctacggaccg | |
| | gccacc | |
| SP0321 | taagtccgggcagggtcctgtccataaaaggcttttcccgggccggctccccgccggcag | 392 |
| (SEQ ID NO: 66) | cgtgccccgccccggcccgctccatctccaaagcatgcagagaatgtctcggcagcccc | |
| | ggtagactgctccaacttggtgtctttccccaaatatggagcctgtgtggagtcactggggg | |
| | agccgggggggggagcggagccggcttcctctagaggtccctatatggttgtgttagagt | |
| | gaacggccagcttcagcccgtctttgctccttgtttgggaagcgagtgggaggggatcaga | |
| | gcaaggggctatataacccttcagcgttcagcctcccgggacaccacccacccagagtg | |
| | gagaagcccagccagtcgctgtcagccacc | |

TABLE 2
CRMs from synthetic promoters of Table 1

| | | |
|---|---|---|
| CRM from SP0497, | tctgagggagacagggaggcatgatcactgccaaatgcccaccaaggacaag | |
| SP0499 and SP0512 | gcacatcccagggagacagacgcagacctggtgccctctggacactggcattc | 159 |
| (SEQ ID NO: 30) | ctggagttctcctctataaatacccgctctggtatttggggttggcagctgttg | |
| CRM_SP0498 | tctgagggagacagggaggcatgatcactgccaaatgcccaccaaggacaag | 183 |
| (SEQ ID NO: 31) | gcacatcccagggagacagacgcagacctggtgccctctggacactggcattc | |
| | ctggagagggtcagtgtcctgccccaacccatgagatgacagactataatagcc | |
| | acaggattaacatagcaggcattg | |
| CRM_SP0500 | ccagcccacctgtcccaatgctgacttagtgcaaggcgagccagcaaggaggg | 192 |
| (SEQ ID NO: 32) | aggacaggtggcagtggggggtgaggagcatctaaaaatagccccagcccac | |
| | ctgtcccaatgctgacttagtgcaaggcgagccagcaaggagggaggacaggt | |
| | ggcagtggggggtgaggagcatctaaaaatagcc | |
| CRM_SP0501 | ccagcccacctgtcccaatgctgacttagtgcaaggcgagccagcaaggaggg | 144 |
| (SEQ ID NO: 33) | aggacaggtggcagtggggggtgaggagcatctaaaaatagccttctcctctata | |
| | aatacccgctctggtatttggggttggcagctgttg | |
| CRM from SP0502, | ctagactagcatgctgcccatgtaaggaggcaaggcctggggacacccgagat | 176 |
| SP0515, SP0521 and | gcctggttataattaacccagacatgtggctgcccccccccccccaacacctgct | |
| SP4169 | gcctctaaaaataaccctgcttctcctctataaatacccgctctggtatttggggttgg | |
| (SEQ ID NO: 34) | cagctgttg | |
| CRM from SP0503 and | tcgtcccctggctggcccatgtaatctgagcccagcattgtacatatcctgggaac | 183 |
| SP0516 | agctgacaatgcagtggtcagacagctggtggggccagctagagctggcaggg | |
| (SEQ ID NO: 35) | ttggctgggaggggagtgtaggctgattctcctctataaatacccgctctggtatttg | |
| | gggttggcagctgttg | |
| CRM from SP0504 and | ttggcccaggtcacactggggtgaggctagtgttcctgagccttgacaaggagac | 190 |
| SP0517 | agcttgaaatagacgagtgtcacatttctgagcagctgtgtggcgacagcaggag | |
| (SEQ ID NO: 36) | gggtagggaatagacagtataaaagagaaagcttctcctctataaatacccgctc | |
| | tggtatttggggttggcagctgttg | |
| CRM from SP0505 and | ccctgccatcttgggtttcagggcagaggagtcttgctaattttgatgcctatttttgga | 194 |
| SP0518 | cacttcagctgccactggctccttataaacgcatgacaccccatgcaaacacact | |
| (SEQ ID NO: 37) | acccctccctccactgctgacaggtgtgtggttctcctctataaatacccgctctggt | |
| | atttggggttggcagctgttg | |
| CRM_SP0506 | aattatttttaataacacttactggtaagagaaaggggagaaaccttagacaggc | 199 |
| (SEQ ID NO: 38) | acttagatgtgactaaggcaggtttatctctgattccaaagcactggagtggaagtc | |
| | acaccgtgactcagagcattgtgatgggccagctgtccattctcctctataaatacc | |
| | cgctctggtatttggggttggcagctgttg | |
| CRM_SP0507 | cagtgttcctatatttatcccaccatacagagcttcctttgcctcagaaggaccagc | 199 |
| (SEQ ID NO: 39) | agtttcgctagcttaacaaaaccagccactcagggtattggtttacagtcaagcaa | |
| | ctctgggagagggcagctgctctcagacatcatacagcttctcctctataaatacc | |
| | cgctctggtatttggggttggcagctgttg | |
| CRM_SP0508 | cttctcaagccaaaggagcaagagttaaaaataacaggctcaccctggcagcc | 165 |
| (SEQ ID NO: 40) | acctgtgctggccagccccaccccatccctccctcggggacagctgcagctcctc | |
| | aggccccgcccgggacattttgggaacactttctcctcttacttctcatcttcaggg | |
| CRM from SP0509 and | agggtcagtgtcctgccccaacccatgagatgacagactataatagccacagga | 200 |
| SP0520 | ttaacatagcaggcattgatttttaaagactgaggaattaggcacctgtcatttttgcc | |
| (SEQ ID NO: 41) | agctggtgtagatgttaaaaattactgtcactcttccgcctgctactttattttgcacct | |
| | gctgttacttgagttacaggcatttca | |
| CRM_SP0510 | atttttaaagactgaggaattaggcacctgtcatttttgccagctggtgtagatgttaa | 176 |
| (SEQ ID NO: 42) | aaattactgtcactcttccgcctgctactttattttgcacctgctgttacttgagttacag | |
| | gcatttcattctcctctataaatacccgctctggtatttggggttggcagctgttg | |
| CRM_SP0511 | agactggggcaggtgcaggctggattgggtttccagaggctatatatataaaggc | 215 |
| (SEQ ID NO: 43) | tgccgggagccccagggccgctccctgagggcacaacactgtgggggcccag | |
| | ccaggcccacattcctttccagaggccagctctccatttatagcccctgggcagag | |
| | cagcttctcctctataaatacccgctctggtatttggggttggcagctgttg | |
| CRM_SP0513 | ccagcccacctgtcccaatgctgacttagtgcaaggcgagccagcaaggaggg | 216 |
| (SEQ ID NO: 44) | aggacaggtggcagtggggggtgaggagcatctaaaaatagccagggtcagt | |
| | gtcctgccccaacccatgagatgacagactataatagccacaggattaacatag | |
| | caggcattgttctcctctataaatacccgctctggtatttggggttggcagctgttg | |
| CRM_SP0514 | ccagcccacctgtcccaatgctgacttagtgcaaggcgagccagcaaggaggg | 192 |
| (SEQ ID NO: 45) | aggacaggtggcagtggggggtgaggagcatctaaaaatagccttctcctctata | |
| | aatacccgctctggtatttggggttggcagctgttgttctcctctataaatacccgctct | |
| | ggtatttggggttggcagctgttg | |
| CRM_SP0519 | cttctcaagccaaaggagcaagagttaaaaataacaggctcaccctggcagcc | 213 |
| (SEQ ID NO: 46) | acctgtgctggccagccccaccccatccctccctcggggacagctgcagctcctc | |
| | aggccccgcccgggacattttgggaacactttctcctcttacttctcatcttcagggtt | |
| | ctcctctataaatacccgctctggtatttggggttggcagctgttg | |
| CRM from SP0522, | gctgcccatgtaaggaggcaaggcctggggacacccgagatgcctggttataat | 162 |
| SP0523 and SP0524 | taacccagacatgtggctgccccccccccccaacacctgctgcctctaaaaata | |
| (SEQ ID NO: 47) | accctgttctcctctataaatacccgctctggtatttggggttggcagctgttg | |

TABLE 3
CREs from synthetic promoters of Table 1

| | | |
|---|---|---|
| CRE0119 | aattatttttaataacacttactggtaagagaaaggggagaaaccttagacaggcacttagat | 151 |
| (SEQ ID NO: | gtgactaaggcaggtttatctctgattccaaagcactggagtggaagtcacaccgtgactca | |
| 48) | gagcattgtgatgggccagctgtcca | |
| CRE0127 | ttggcccaggtcacactggggtgaggctagtgttcctgagccttgacaaggagacagcttga | 142 |
| (SEQ ID NO: | aatagacgagtgtcacatttctgagcagctgtgtggcgacagcaggaggggtagggaata | |
| 49) | gacagtataaaagagaaagc | |
| CRE0137 | ccctgccatcttgggtttcagggcagaggagtcttgctaattttgatgcctatttttggacacttca | 146 |
| (SEQ ID NO: | gctgccactggctccttataaacgcatgacaccccatgcaaacacactacccctccctccac | |
| 50) | tgctgacaggtgtgtgg | |
| CRE0138 | cttctcaagccaaaggagcaagagttaaaaataacaggctcaccctggcagccacctgtg | 165 |
| (SEQ ID NO: | ctggccagccccaccccatccctccctcggggacagctgcagctcctcaggccccgcccg | |
| 51) | ggacattttgggaacactttctcctcttacttctcatcttcaggg | |
| CRE0139 | cagtgttcctatatttatcccaccatacagagcttcctttgcctcagaaggaccagcagtttcgc | 151 |
| (SEQ ID NO: | tagcttaacaaaaccagccactcagggtattggtttacagtcaagcaactctgggagaggg | |
| 52) | cagctgctctcagacatcatacagc | |
| CRE0143 | tcgtcccctggctggcccatgtaatctgagcccagcattgtacatatcctgggaacagctgac | 135 |
| (SEQ ID NO: | aatgcagtggtcagacagctggtggggccagctagagctggcagggttggctgggaggg | |
| 53) | gagtgtaggctga | |
| CRE0145 | gctgcccatgtaaggaggcaaggcctggggacacccgagatgcctggttataattaaccc | 114 |
| (SEQ ID NO: | agacatgtggctgccccccccccccaacacctgctgcctctaaaaataaccctg | |
| 54) | | |
| CRE0077 | tctgagggagacagggaggcatgatcactgccaaatgcccaccaaggacaaggcacat | 111 |
| (SEQ ID NO: | cccagggagacagacgcagacctggtgccctctggacactggcattcctggag | |
| 55) | | |
| DES_MT_en | ttctcctctataaatacccgctctggtatttggggttggcagctgttg | 48 |
| hancer_48 bp | | |
| (SEQ ID NO: | | |
| 56) | | |
| CRE0075 | agggtcagtgtcctgccccaacccatgagatgacagactataatagccacaggattaacat | 72 |
| (SEQ ID NO: | agcaggcattg | |
| 57) | | |
| CRE0083 | ccagcccacctgtcccaatgctgacttagtgcaaggcgagccagcaaggagggaggaca | 96 |
| (SEQ ID NO: | ggtggcagtggggggtgaggagcatctaaaaatagcc | |
| 58) | | |
| Ch2EnhMYL1_ | atttttaaagactgaggaattaggcacctgtcatttttgccagctggtgtagatgttaaaaattac | |
| 3_v1 | tgtcactcttccgcctgctactttattttgcacctgctgttacttgagttacaggcatttca | |
| (SEQ ID NO: | | |
| 59) | | |
| CRE0050 | ctagactagcatgctgcccatgtaaggaggcaaggcctggggacacccgagatgcctggt | 128 |
| (SEQ ID NO: | tataattaacccagacatgtggctgcccccccccccccaacacctgctgcctctaaaaataa | |
| 60) | ccctgc | |
| CRE0069 | agactggggcaggtgcaggctggattgggtttccagaggctatatatataaaggctgccgg | 167 |
| (SEQ ID NO: | gagccccagggccgctccctgagggcacaacactgtgggggcccagccaggcccacatt | |
| 61) | cctttccagaggccagctctccatttatagcccctgggcagagcagc | |
| CRE0031 | taagtccgggcagggtcctgtccataaaaggcttttcccgggccggctccccgccggcagc | 216 |
| (SEQ ID NO: | gtgccccgccccggcccgctccatctccaaagcatgcagagaatgtctcggcagccccgg | |
| 67) | tagactgctccaacttggtgtctttccccaaatatggagcctgtgtggagtcactgggggagc | |
| | cgggggtggggagcggagccggcttcctctag | |

TABLE 4
Promoter elements from synthetic promoters of Table 1

| | | |
|---|---|---|
| BG_mp | gggctgggcataaaagtcagggcagagccatctattgcttacatttgcttctg | 53 |
| (SEQ ID NO: | | |
| 62) | | |
| SCP1 | gtacttatataagggggtgggggcgcgttcgtcctcagtcgcgatcgaacactcgagccga | 81 |
| (SEQ ID NO: | gcagacgtgcctacggaccg | |
| 63) | | |
| CRE0070 | cggccggggccgcattcctgggggccgggcggtgctcccgcccgcctcgataaaaggctc | 105 |
| (SEQ ID NO: | cggggccggcggcggcccacgagctacccggaggagcgggaggcg | |
| 64) | | |
| CRE0053 | caccgcctgctgccacggccggccgtataaatagaggcgaggagcagctgggctctcttg | 69 |
| (SEQ ID NO: | gcagtcacc | |
| 65) | | |
| CRE0037 | aggtccctatatggttgtgttagagtgaacggccagcttcagcccgtctttgctccttgtttggga | 176 |
| (SEQ ID NO: | agcgagtgggaggggatcagagcaaggggctatataacccttcagcgttcagcctcccgg | |
| 68) | gacaccacccacccagagtggagaagcccagccagtcgctgtcagccacc | |

TABLE 5
Schematic representation of the muscle-specific synthetic
promoters according to embodiments of this invention with
the cis-regulatory elements and promoter elements indicated

| | | | | Promoter |
|---|---|---|---|---|
| | CRE | CRE | CRE | element |
| SP0497 | | CRE0077 | DES_MT_enhancer_48bp | CRE0070 |
| SP0498 | | CRE0077 | CRE0075 | CRE0053 |
| SP0499 | | CRE0077 | DES_MT_enhancer_48bp | CRE0053 |
| SP0500 | | CRE0083 | CRE0083 | CRE0053 |
| SP0501 | | CRE0083 | DES_MT_enhancer_48bp | CRE0053 |
| SP0502 | | CRE0050 | DES_MT_enhancer_48bp | CRE0053 |
| SP0503 | | CRE0143 | DES_MT_enhancer_48bp | CRE0053 |
| SP0504 | | CRE0127 | DES_MT_enhancer_48bp | CRE0053 |
| SP0505 | | CRE0137 | DES_MT_enhancer_48bp | CRE0053 |
| SP0506 | | CRE0119 | DES_MT_enhancer_48bp | CRE0053 |
| SP0507 | | CRE0139 | DES_MT_enhancer_48bp | CRE0053 |
| SP0508 | | | CRE0138 | CRE0053 |
| SP0509 | | CRE0075 | Ch2EnhMYL1_3_v1 | CRE0053 |
| SP0510 | | Ch2EnhMYL1_3_v1 | DES_MT_enhancer_48bp | CRE0053 |
| SP0511 | | CRE0069 | DES_MT_enhancer_48bp | BG mp |
| SP0512 | | CRE0077 | DES_MT_enhancer_48bp | BG mp |
| SP0513 | CRE0083 | CRE0075 | DES_MT_enhancer_48bp | BG mp |
| SP0514 | CRE0083 | DES_MT_enhancer_48bp | DES_MT_enhancer_48bp | BG mp |
| SP0515 | | CRE0050 | DES_MT_enhancer_48bp | BG mp |
| SP0516 | | CRE0143 | DES_MT_enhancer_48bp | BG mp |
| SP0517 | | CRE0127 | DES_MT_enhancer_48bp | BG mp |
| SP0518 | | CRE0137 | DES_MT_enhancer_48bp | BG mp |
| SP0519 | | CRE0138 | DES_MT_enhancer_48bp | BG mp |
| SP0520 | | CRE0075 | Ch2EnhMYL1_3_v1 | BG mp |
| SP0521 | | CRE0050 | DES_MT_enhancer_48bp | SCP1 |
| SP4169 | | CRE0050 | DES_MT_enhancer_48bp | CRE0070 |
| SP0522 | | CRE0145 | DES_MT_enhancer_48bp | CRE0053 |
| SP0523 | | CRE0145 | DES_MT_enhancer_48bp | BG mp |
| SP0524 | | CRE0145 | DES_MT_enhancer_48bp | SCP1 |
| SP0321 | | | CRE0031 | CRE0037 |

1. A synthetic muscle-specific promoter comprising:
a) a sequence according to any one of SEQ ID NOs: 1-29, 66 or a functional variant thereof; or
b) a cis-regulatory module (CRM) comprising a sequence according to any one of SEQ ID NOs: 30-47 or a functional variant thereof.

2. The synthetic muscle-specific promoter of claim 1 a) comprising a sequence which is at least 70%, 75%, 80%, 85%, 90%, 95%, 96%, 97%, 98% or 99% identical to any one of SEQ ID NOs: 1-29, 66.

3. The synthetic muscle-specific promoter of claim 1 b) wherein the CRM comprises a sequence which is at least 70%, 75%, 80%, 85%, 90%, 95%, 96%, 97%, 98% or 99% identical to any one of SEQ ID NOs: 30-47.

4. The synthetic muscle-specific promoter of claim 3 comprising said CRM as set out above operably linked to a promoter element.

5. The synthetic muscle-specific promoter of any preceding claim wherein the functional variant retains at least 25%, 50%, 75%, 80%, 85%, 90%, 95% or 100% of the activity of the reference promoter.

6. A muscle-specific cis-regulatory element (CRE) comprising a sequence according to any one of SEQ ID NOs: 48-61, 67 or a functional variant of any thereof.

7. The muscle-specific CRE of claim 6 comprising a sequence which is at least 70%, 75%, 80%, 85%, 90%, 95%, 96%, 97%, 98% or 99% identical to any one of SEQ ID NOs: 48-61, 67.

8. A synthetic muscle-specific promoter comprising a CRE according to claim 6 or 7.

9. An isolated minimal or proximal promoter comprising a sequence according to any one of SEQ ID NOs: 62-65, 68 or a functional variant thereof.

10. An isolated minimal or proximal promoter according to claim 9 comprising a sequence which is at least 70%, 75%, 80%, 85%, 90%, 95%, 96%, 97%, 98% or 99% identical to SEQ ID NO: 62-65, 68.

11. A synthetic muscle-specific promoter comprising a minimal or proximal promoter according to claim 9 or 10.

12. An expression cassette comprising a synthetic muscle-specific promoter according to any one of claims 1 to 5, 8 and 11 operably linked to a sequence encoding an expression product.

13. A vector comprising a synthetic muscle-specific promoter according to any one of claims 1 to 5, 8 and 11 or an expression cassette according to claim 12.

14. The vector of claim 13, which is an AAV vector, an adenoviral vector, a retroviral vector or a lentiviral vector.

15. A virion comprising a vector according to claim 14.

16. A pharmaceutical composition comprising a synthetic muscle-specific promoter according to any one of claims 1 to 5, 8 and 11, expression cassette according to claim 12, vector according to claim 13 or 14, or virion according to claim 15.

17. A synthetic muscle-specific promoter according to any one of claims 1 to 5, 8 and 11, expression cassette according to claim 12, vector according to claim 13 or 14, virion according to claim 15, or pharmaceutical composition according to claim 16 for use in therapy.

18. A cell comprising a synthetic muscle-specific promoter according to any one of claims 1 to 5, 8 and 11, expression cassette according to claim 12, vector according to claim 13 or 14, virion according to claim 15.

19. A synthetic muscle-specific promoter according to any one of claims 1 to 5, 8 and 11, expression cassette according to claim 12, vector according to claim 13 or 14, virion according to claim 15 or pharmaceutical composition according to claim 16 for use in the manufacture of a pharmaceutical composition for the treatment of a medical condition or disease.

20. A method for producing an expression product, the method comprising providing a synthetic muscle-specific expression cassette according to claim 12 in a muscle cell and expressing the gene present in the synthetic muscle-specific expression cassette.

21. A method of expressing a therapeutic transgene in a muscle cell, the method comprising introducing into the muscle cell a synthetic muscle-specific expression cassette according to claim 12, vector according to claim 13 or 14, virion according to claim 15.

22. A method of therapy of a subject, preferably a human, in need thereof, the method comprising:
administering to the subject an expression cassette according to claim 12, vector
according to claim 13 or 14, virion according to claim 15 or pharmaceutical composition
according to claim 16, which comprises a sequence encoding a therapeutic product
operably linked to a promoter according any one of claims 1 to 5, 8 and 11; and
expressing a therapeutic amount of the therapeutic product in the muscle of said subject.

23. The method of therapy of a subject of claim 22, wherein the therapeutic amount of the therapeutic product is expressed in the skeletal and/or cardiac muscle.
